(19) United States
(12) Patent Application Publication
(10) Pub. No.: US 2016/0160263 A1
(43) Pub. Date: Jun. 9, 2016

(54) PERSONALIZED PROTEASE ASSAY TO MEASURE PROTEASE ACTIVITY IN NEOPLASMS

(71) Applicant: The Regents of the University of California, Oakland, CA (US)

(72) Inventors: Mike Whitney, San Diego, CA (US); Quyen T. Nguyen, La Jolla, CA (US); Roger Y. Tsien, La Jolla, CA (US)

(21) Appl. No.: 14/874,297

(22) Filed: Oct. 2, 2015

Related U.S. Application Data
(60) Provisional application No. 62/059,081, filed on Oct. 2, 2014.

(51) Int. Cl. C12Q 1/37

(52) U.S. Cl. CPC C12Q 1/37

(57) ABSTRACT

Disclosed herein, the invention pertains to methods and compositions that find use in diagnostic, prognostic and characterization of neoplasia samples based on the ability of a neoplasia sample to cleave a MTS molecule of the present invention. In some embodiments, a MTS molecule disclosed herein has the formula (A-X-B-C), wherein A is a peptide with a sequence comprising 5 to 9 consecutive acidic amino acids, wherein the amino acids are selected from: aspartates and glutamates; B is a peptide with a sequence comprising 5 to 20 consecutive basic amino acids; X is a linker; and C is a detectable moiety.

### CROSS REFERENCE TO RELATED APPLICATIONS

This application claims priority to U.S. Provisional Application No. 62/059,081, filed on Oct. 2, 2014, and which is incorporated by reference herein in its entirety for all purposes.

### STATEMENT OF FEDERALLY-SPONSORED RESEARCH

This work was supported in part by grants from the Howard Hughes Medical Institute, the Department of Defense (W81XWH-09-1-0699), National Cancer Institute (CA158448-01 and P50 CA 097007-DRP), NIBIB (K08 EB008122-01), and Burroughs Wellcome Fund. The government has certain rights in this invention.

### FIELD OF THE INVENTION

This invention pertains to methods and composition that find use in diagnostic, prognostic and characterization of neoplasia samples based on the ability of a neoplasia sample to cleave a MTS molecule of the present invention.

### BACKGROUND OF THE INVENTION

### Introduction

Cell membranes delimit the outer boundaries of cells, and regulate transport into and out of the cell interior. Made primarily of lipids and proteins, they provide a hydrophilic surface enclosing a hydrophobic interior across which materials must pass before entering a cell. Although many small, lipophilic compounds are able to cross cell membranes passively, most compounds, particles and materials must rely on active mechanisms in order to gain entry into a living cell.

### Transmembrane Transport

Regulation of transport into and out of a cell is vital for its continued viability. For example, cell membranes contain ion channels, pumps, and exchangers capable of facilitating the transmembrane passage of many important substances. However, transmembrane transport is selective: in addition to facilitating the entry of desired substances into a cell, and facilitating the exit of others, a major role of a cell membrane is to prevent uncontrolled entry of substances into the cell interior. This barrier function of the cell membrane makes difficult the delivery of markers, drugs, nucleic acids, and other exogenous material into cells.

Over the last decade, peptide sequences that can readily enter a cell have been identified. For example, the Tat protein of the human immunodeficiency virus 1 (HIV-1) is able to enter cells from the extracellular environment (e.g., Fawell et al. P.N.A.S. 91:664-668 (1994)). Such uptake is reviewed in, for example, Richard et al., J. Biol. Chem. 278(1):585-590 (2003).

Such molecules that are readily taken into cells may also be used to carry other molecules into cells along with them. Molecules that are capable of facilitating transport of substances into cells have been termed “membrane translocation signals” (MTS) as described in Tung et al., Advanced Drug Delivery Reviews 55:281-294 (2003). The most important MTS are rich in amino acids such as arginine with positively charged side chains. Molecules transported into cell by such cationic peptides may be termed “cargo” and may be reversibly or irreversibly linked to the cationic peptides. An example of a reversible linkage is found in Zhang et al., P.N.A.S. 95:9184-9189 (1994)).

MTS molecules are discussed in, for example, Wender et al., P.N.A.S. 97:13003-13008 (2000); Hallbrink et al., Biochim. Biophys. Acta 1515:101-109 (2001); Derossi et al., Trends in Cell Biology 8:84-87 (1998); Rothbard et al., J. Med. Chem. 45:3612-3618 (2002); Rothbard et al., Nature Medicine 6(11):1253-1247 (2000); Wadia et al., Curr. Opinion Biotech. 13:52-56 (2002); Futaki et al.; Bioconj. Chem. 12:1005-1011 (2001); Rothbard et al., U.S. Pat. No. 6,306,993; Frankel et al., U.S. Pat. No. 6,316,003; Rothbard et al., U.S. Pat. No. 6,495,663; Monahan et al., U.S. Pat. No. 6,630,351 and Jiang et al., WO 2005/042034.

### Cancer Surgery

In cancer surgery, positive margins, defined as tumor cells present at the cut edge of the surgical specimen, have been associated with increased local recurrence and a poor prognosis (Hague R., et al., BMC Ear Nose Throat Disord. 16:2 (2006)). As in most solid tumors, salvage surgery (i.e., re-excision of the positive margin) or adjuvant chemotherapy and/or radiation not only cause extra trauma and expense but also often fail to remediate the poor outcome (Hague R., et al., BMC Ear Nose Throat Disord. 16:2 (2006); Singletary S. Am. J. Surg. 184:383-393 (2002); Meric F., et al., Cancer 97:926-933 (2003); Snijder R., et al., Annals of Thoracic Surg. 65 (1998); Nagtegaal I D, Quirke P., J. Clin. On. 26:303-312 (2008); Dotan Z, et al., J. Urol. 178:2308-2312 (2007); and Wieder J.A., J. Urol. 160:299-315 (1998)).

The reason for this observation is likely multifactorial and related in part to the difficulty in identifying the residual cancer during repeat surgery. Therefore, development of more sensitive imaging and diagnostic assays for more accurate detection of positive surgical margins during the primary operation would be one of the most effective means to minimize patient suffering and expense and to improve survival.

### Role of MMPs in Cancer

MMPs play crucial roles in cancer invasion and metastasis (Bauvois B. et al. Biochim Biophys Acta. 1825:29-36 (2012)) are overexpressed malignant tumors and their expression/activity is associated with \poor patient prognosis. Increased MMP expression has been shown to correlate with cancer grade (Wittekindt C., et al. Acta Otolaryngol. 131:101-106 (2011)) and decreased survival (Liu W. W., et al. Otolaryngol Head Neck Surg. 132:395-400 (2005) and Mallis A., et al., Eur Arch Otorhinolaryngol. 269:639-642 (2012)). In carcinoma of the tongue, increased MMP expression has been shown to correlate with incidence of lymph node metastases (Zhou, C. X., et al., Aust Dent J. 55:385-389 (2010)).

### Heterogeneity/Specificity

Although increased MMP expression has been shown to correlate with increased cancer grade and stage and decreased survival (Wang W L, et al., Mol Carcinog. 2012 and P. O. C., Arch Otolaryngol Head Neck Surg. 127:813-820 (2001)), there is significant heterogeneity that exists between patients in terms of absolute MMP levels (Wang W L, et al., Mol Carcinog. 2012). This invention describes a method to address this heterogeneity and evaluate the clinical utility of ACPPs in cancers from multiple body sites using an ex-vivo screening assay to determine MMP activity for individual human cell line derived and surgical tumor samples. MMP activity can also be elevated at some sites of nonmalignant inflammation, such as skin lacerations and atherosclerotic plaques (Olson E.S., et al., Integr Biol (Camb). (2012)), but these are anatomically remote from and easily distinguished intraoperatively from tumor margins and potentially metastatic lymph nodes. In our experience, such other sites of MMP activity are unlikely to confuse any experienced clinician, just as the enormous ¹⁸F signal in normal brain, heart, and bladder during [¹⁸F]-FDG PET scans does not prevent the usefulness of such imaging in locating tumors and metastases with high glucose utilization. Because of the concern that MMP expression is also increased in inflammation/wound healing, part of the study is to evaluate the threshold of MTS (activatable cell penetrating peptides; ACPP) uptake that can reliably distinguish cancer from non-cancer tissue. Besides MMPs which were the focus of initial studies, MTS have been developed the target other proteases that have been proposed to be involved in cancer including, elastases, thrombin, plasmin, legumain, cathepsins.

All patents and publications, both supra and infra, are hereby incorporated by reference in their entirety.

As the field of molecularly targeting fluorescent markers for early cancer detection and intraoperative margin evaluation progresses and more enzymatically activatable probes (Jiang T., et al. Proc Natl Acad Sci USA. 101:17867-17872 (2004); Aguilera T.A., et al., Integr. Biol. 1:371-381 (2009); Olson E.S., et al., Integr Biol (Camb). 1:382-393 (2009); Olson E.S., et al., Proc Natl Acad Sci USA. 107:4311-4316 (2010); Nguyen Q. T., Proc Natl Acad Sci USA. 107:4317-4322 (2010); Blum G., et al., Nat Chem Biol. 3:668-677 (2007); Gounaris E., et al., PLoS One. 3:e2916 (2008); Bremer C., et al., Invest Radiol. 40:321-327 (2005)) are becoming available for clinical use, methods such as a personalized protease assay (PePA) would be useful in a variety of diagnostic and prognostic applications.

As such, there remains a need in the art for additional diagnosis, prognosis and characterization, including development personalized protease assays, useful in both in vivo and ex vivo applications. Such methods would allow for the development of better and more personalized treatment regimens. The present invention meets these needs and provides methods for ex vivo diagnosis, prognosis and characterization of tumors which can find use in a variety of personalized medicine applications.

### SUMMARY OF THE INVENTION

Methods of use and compositions comprising MTS molecules are disclosed. MTS molecules having features of the invention include peptide portions linked by a cleavable linker portion which may be a peptide. The inventors have found that these MTS molecules can find use in diagnostic, prognostic and characterization assays.

In some embodiments, the present invention provides an ex vivo method for detecting the presence of one or more protease activities in a neoplasia sample comprising a) combining ex vivo said neoplasia sample from a subject with a molecule of the structure A-X-B-C, wherein B is a peptide portion of about 5 to about 20 basic amino acid residues, which is suitable for cellular uptake, A is a peptide portion of about 2 to about 20 acidic amino acid residues, which when linked with portion B is effective to inhibit or prevent cellular uptake of portion B, and X is a cleavable linker of about 2 to about 100 atoms joining A with B, where X is cleavable under physiological conditions, and C is a detectable moiety; and b) detecting cleavage of A-X-B-C by detecting a change in said detectable moiety C, wherein said change in C is indicative of cleavage and said cleavage is indicative of the presence of one or more protease activities in said neoplasia.

In some embodiments, the present invention provides an ex vivo method of determining a treatment regimen based on the protease profile of a neoplasia sample, comprising a) combining ex vivo said neoplasia sample from a subject with a molecule of the structure A-X-B-C, wherein B is a peptide portion of about 5 to about 20 basic amino acid residues, which is suitable for cellular uptake, A is a peptide portion of about 2 to about 20 acidic amino acid residues, which when linked with portion B is effective to inhibit or prevent cellular uptake of portion B, and X is a cleavable linker of about 2 to about 100 atoms joining A with B, where X is cleavable under physiological conditions and C is a detectable moiety; and b) detecting cleavage of A-X-B-C by detecting a change in detectable moiety C, wherein said change in C is indicative of cleavage and said cleavage is indicative of the presence of one or more protease activities and wherein the presence and/or absence of one or more protease activities allows for determining a medical treatment regimen.

In some embodiments, the medical regimen is a surgical regimen.

In some embodiments, the protease activity is indicative of neoplasia. In some embodiments, the protease activity is indicative of metastasis.

In some embodiments, C is a fluorescent detectable moiety.

In some embodiments, C comprises a FRET pair.

In some embodiments, the molecule of the invention further comprises a Q moiety, wherein when said Q moiety is present, said molecule has the structure Q-A-X-B-C.

In some embodiments, the method of any of the preceding claims wherein C and Q comprise a FRET pair. In some embodiments, the FRET pair is selected from the group consisting of CFP:YFP; Cy5:Cy7; FITC:TRITC; Cy3:Cy5; EGFP:Cy3; EGFP:YFP; 6-FAM:LC Red 640 or Alexa Fluor 546; fluorescein:tetramethylrhodamine; IAEDANS:fluorescein; EDANS:Dabcyl; fluorescein:fluorescein; BODIPY FL:BODIPY FL; and fluorescein:QSY 7 and QSY 9.

In some embodiments, cleavage of A-X-B-C is detected by FRET.

In some embodiments, cleavage of Q-A-X-B-C is detected by FRET.

In some embodiments, the peptide portion A comprises about 5 to about 9 glutamates or aspartates. In some embodiments, the peptide portion A comprises about 5 to about 9 consecutive glutamates or aspartates. In some embodiments, the peptide portion B comprises about 9 to about 16 arginines. In some embodiments, the peptide portion B comprises about 9 to about 16 consecutive arginines.

In some embodiments, the peptide portion A comprises D-amino acids. In some embodiments, the peptide portion B comprises D-amino acids. In some embodiments, the peptide portion A consists of D-amino acids. In some embodiments, the peptide portion B consists of D-amino acids. In some embodiments, the peptide portions A and B consists of D-amino acids.

In some embodiments, the cleavable linker X is a flexible linker. In some embodiments, the cleavable linker X is a flexible linker about 6 to about 30 atoms in length.

In some embodiments, the cleavable linker X is cleavable in an acidic environment.

In some embodiments, the cleavable linker X comprises a peptide linkage.

In some embodiments, the cleavable linker X comprises aminocaproic acid.

In some embodiments, the cleavable linker X is configured for cleavage by an enzyme. In some embodiments, the enzyme is selected from the group consisting of a matrix metalloprotease, elastase, plasmin, thrombin, chymase, urokinase-type plasminogen activator and tissue plasminogen activator. In some embodiments, the cleavable linker X comprises an amino acid sequence selected from the group consisting of PLGLAG (SEQ ID NO: 1), PLGC(met)AG (SEQ ID NO: 2), EDDDDKA (SEQ ID NO: 3), RS-(Cit)-G-(homoF)-YLY (SEQ ID NO: 4), CRPAHLRDSG (SEQ ID NO: 5), SLAYYTA (SEQ ID NO: 6), NISDLTAG (SEQ ID NO: 7), PPSSLRVT (SEQ ID NO: 8), SGESLSNLTA (SEQ ID NO: 9), RIGFLR (SEQ ID NO: 10), RLQLA(acetyl)L (SEQ ID NO: 11), RLQLKL (SEQ ID NO: 12), DPRSFL (SEQ ID NO: 13), PPRSFL (SEQ ID NO: 14), Norleucine-TPRSFL (SEQ ID NO: 15), GVAY|SGA (SEQ ID NO: 16), YGRAAA (SEQ ID NO: 17), YGPRNR (SEQ ID NO: 18), RSHP(Hfe)TLY (SEQ ID NO: 19), RSHG(Hfe)FLY (SEQ ID NO: 20), SNPYK-Y (SEQ ID NO: 21), SNPKG-Y (SEQ ID NO: 22), SNPYG-Y (SEQ ID NO: 23), TLSE-LH (SEQ ID NO: 24), TIAHLA (SEQ ID NO: 25), (RLQLK(acetyl)L (SEQ ID NO: 26), and KLRFSKQ (SEQ ID NO: 27).

In some embodiments, the cleavable linker X comprises a S-S linkage.

In some embodiments, the cleavable linker X comprises a transition metal complex, wherein said transition metal complex linker is cleaved when the metal is reduced.

In some embodiments, the method comprises multiple molecules of the structure A-X-B-C and wherein the cleavable linker X comprises a plurality of cleavable linkers X. In some embodiments, the plurality of cleavable linkers X linking a portion A to a structure B-C are cleavable by a single protease. In some embodiments, the plurality of cleavable linkers X linking a portion A to a structure B-C are cleavable by more than one protease.

In some embodiments, the method comprises an in vivo method of determining a treatment regimen based on the protease profile of a neoplasia, comprising a) providing to a subject a molecule of the structure A-X-B-C, wherein B is a peptide portion of about 5 to about 20 basic amino acid residues, which is suitable for cellular uptake, A is a peptide portion of about 2 to about 20 acidic amino acid residues, which when linked with portion B is effective to inhibit or prevent cellular uptake of portion B, and X is a cleavable linker of about 2 to about 100 atoms joining A with B, where X is cleavable under physiological conditions and C is a detectable moiety; and b) detecting cleavage of A-X-B-C by detecting a change in detectable moiety C, wherein said change in C is indicative of cleavage and said cleavage is indicative of the presence of one or more protease activities and wherein the presence and/or absence of one or more protease activities allows for determining a medical treatment regimen.

In some embodiments, the medical treatment regimen is a surgical regimen.

In some embodiments, the presence of the protease activity is indicative of neoplasia.

In some embodiments, the cleavable linker X comprises an amino acid sequence selected from the group consisting of PLGLAG (SEQ ID NO: 1), PLGC(met)AG (SEQ ID NO: 2), EDDDDKA (SEQ ID NO: 3), RS-(Cit)-G-(homoF)-YLY (SEQ ID NO: 4), CRPAHLRDSG (SEQ ID NO: 5), SLAYYTA (SEQ ID NO: 6), NISDLTAG (SEQ ID NO: 7), PPSSLRVT (SEQ ID NO: 8), SGESLSNLTA (SEQ ID NO: 9), RIGFLR (SEQ ID NO: 10), RLQLA(acetyl)L (SEQ ID NO: 11), RLQLKL (SEQ ID NO: 12), DPRSFL (SEQ ID NO: 13), PPRSFL (SEQ ID NO: 14), Norleucine-TPRSFL (SEQ ID NO: 15), GVAY|SGA (SEQ ID NO: 16), YGRAAA (SEQ ID NO: 17), YGPRNR (SEQ ID NO:18), RSHP(Hfe)TLY (SEQ ID NO: 19), RSHG(Hfe)FLY (SEQ ID NO: 20), SNPYK-Y (SEQ ID NO: 21), SNPKG-Y (SEQ ID NO: 22), SNPYG-Y (SEQ ID NO: 23), TLSE-LH (SEQ ID NO: 24), TIAHLA (SEQ ID NO: 25), (RLQLK(acetyl)L (SEQ ID NO: 26), and KLRFSKQ (SEQ ID NO: 27).

### BRIEF DESCRIPTION OF THE DRAWINGS

FIG. 1 describes that the ability to cleave RACPPs (radiometric MTSs) to be assayed ex vivo on frozen tissue samples and may differentiate normal from tumor tissues. Y axis indicates rates of change of Cy5 fluorescence (arbitrary units) over time following addition of MMP- and elastase-sensitive RACPPs to 100 mg homogenized fatty tissue from mouse, normal human breast, two mouse breast cancer grafts (4T1 and 8119), and several human head and neck squamous cell carcinoma surgical specimens.

FIG. 2A describes Higher MMP expression in tumors versus normal tissue in TCGA HNSCC. FIG. 2B HPV+ tumors have lower MMP expression than HPV-tumors.

FIG. 2C and FIG. 2D Higher MMP-2/MMP-14 expression in HPV+ tumors correlates with poorer prognosis. Abbreviations: MMP, matrix-metalloproteinase; TCGA, the Cancer Genomic Atlas; HNSCC, head and neck squamous cell carcinoma; HPV, human papilloma virus.

FIG. 3 describes RACPP schematic showing (A) no tumor-contrast immediately post-injection; (B) high tumor-contrast following MMP-dependent cleavage, separating Cy5 from Cy7. (C) Application of RACPP to HNSCC specimens produces faster Cy5/Cy7 ratio-change compared to normal tissue. Abbreviations: RACPP, ratiometric activatable cell-penetrating peptide; HNSCC, head and neck squamous cell carcinoma.

FIG. 4 describes (A, B) RACPP injection produces greater ratiometric fluorescent signal in HNSCC tumor versus normal tissue. (C) Uncleavable-control does not produce tumor-specific contrast. (D) RACPP is sensitive and specific for tumor detection. Abbreviations: HNSCC, head and neck squamous cell carcinoma; RACPP, ratiometric activatable cell-penetrating peptide.

FIG. 5 describes (A) Ratiometric images showing higher fluorescence in tumor (white stippling). (B) Corresponding H&E images confirming tumor burden (red stippling). (C) Ratiometric activatable cell-penetrating peptide (RACPP) uptake correlates directly with tumor burden.

FIG. 6 describes select ACPP substrates.

FIG. 7 describes selectivity for substrates for MMP2, 9 and 14 (1 uM peptide 20 nM enzyme). A) MT1 selective RSHPHfeTLY (SEQ ID NO: 19). B) MMP2 selective TIAHLA (SEQ ID NO: 25). C) MMP9 selective SNPYKY (SEQ ID NO: 21).

FIG. 8A, FIG. 8B and FIG. 8C describe selectivity for substrate cut with MMP-2, MMP9 and MMP-14. FIG. 8A, FIG. 8B and FIG. 8C describe 1 PLGmetCAG-MMP2 (SEQ ID NO: 28), 9,14, 2 TLSELH-MMP-2 selective (SEQ ID NO: 24), 3 TIAHLA-MMP2 selective (SEQ ID NO: 25), 4 CATK-KLRFSKQ (SEQ ID NO: 29), 5 Cit-MMP14 selective, 6 RSHG(Hfe)FLY-MMP14 (SEQ ID NO: 20) selective, 7 RSHP(Hfe)TLY-MMP14 selective (SEQ ID NO: 19), 8 PLGLEEA-MMP12 (SEQ ID NO:30) selective, and 9 SNPYKY-MMP-9 (SEQ ID NO: 21) selective.

FIG. 9 describes selectivity for substrates with A) Panc2 supernatant no radiation abd B) without MMP-2 substrates.

FIG. 10A, FIG. 10B, FIG. 10C and FIG. 10D describe testing of FRET versions of newly optimized MMP-9 selective substrates. FIG. 10A SNPYK-Y (SEQ ID NO: 21) substrate. FIG. 10B SNPKG-Y (SEQ ID NO: 22) substrate. FIG. 10C SNPYG-Y (SEQ ID NO: 23) substrate. FIG. 10D SNPFKY (SEQ ID NO: 31) substrate.

FIG. 11A, FIG. 11B, FIG. 11C, FIG. 11D, FIG. 11E and FIG. 11F describe MMP2 FRET substrates based on rational substitution of consensus/preferred residues. Peptides include:
MMP2-1=FAM-e9-dPEG(6)-SGTLAH-LHTA-r9-(D-cys)-NH2
MMP2-2=FAM-e9-dPEG(6)-SGTLSE-LHTA-r9-(D-cys)-NH2
MMP2-3=FAM-e9-dPEG(6)-SGTISH-LHTA-r9-(D-cys)-NH2
MMP2-4=FAM-e9-dPEG(6)-SGTLSH-LHTA-r9-(D-cys)-NH2
MMP2-5=FAM-e9-dPEG(6)-SGTIAH-FHTA-r9-(D-cys)-NH2

FIG. 12 describes design, synthesis and testing of new Cathepsin K substrates. FAM-e9-dPEG(6)-XXXXXX-r9-(D-cys)-NH2-general format.

FIG. 13A, FIG. 13B, FIG. 13C, FIG. 13D, FIG. 13E and FIG. 13F describe design, synthesis and testing of new Cathepsin K substrates. FAM/Cy5 FRET versions. FIG. 13A KPRGSKQ (SEQ ID NO: 32) substrate. FIG. 13B KLRFSKQ (SEQ ID NO: 33) substrate. FIG. 13C KKPGSKQ (SEQ ID NO: 34) substrate. FIG. 13D HPGGPQ (SEQ ID NO: 35) substrate. FIG. 13E NleTLRSLQ (SEQ ID NO: 36) substrate.

FIG. 14A, FIG. 14B, FIG. 14C and FIG. 14D describe generation of new FRET (FAM/Cy5) versions of MT1-MMP(MMP-14) selective ACPPs. FIG. 14A O-RSHP(Hfe)TLY-(SEQ ID NO: 19) substrate. FIG. 14B O-RSHG(Hfe)FLY (SEQ ID NO: 20) substrate. FIG. 14C Original-R-S-cit-G-Hfe-YLY (SEQ ID NO: 38) substrate. FIG. 14D dPEG6-SG-ARGIKL-TA (SEQ ID NO: 37) substrate.

FIG. 15 describes making ACPPs that have improved selectivity for specific MMPs-Cy5/Cy7 FRET versions. A) PLGC(Me)AG (SEQ ID NO: 2) substrate (MMP2/9)+. B) RS-cit-G-homoF-YLY (SEQ ID NO: 4) substrate (MT1-MMP). C) Substrate diagram. D) Cy5/Cy7 tumor ratio.

FIG. 16 describes skin off images for Cal-27 tumors 2 hrs post 10 nmole injection. PLG, MT1-MMP “Cit” O-R-S-cit-G-Hfe-YLY (SEQ ID NO: 38), MT1-“New” 0-RSHP(Hfe)TLY-(SEQ ID NO: 19) substrates.

FIG. 17A and FIG. 17B are diagrams of Cy5/Cy7 FRET probes.

FIG. 18A, FIG. 18B, FIG. 18C and FIG. 18D provide additional comparison data for Cy5/Cy7 FRET probes with “branched old” versus “backbone new” peg12. FIG. 18A PLGC(met)AG (SEQ ID NO: 2) (Branched Peg12). FIG. 18B New PLGC(met)AG (SEQ ID NO: 2) (backbone peg12). FIG. 18C Nle-TPRSFL (SEQ ID NO: 15) (Original branched). FIG. 18D CatK (backbone peg6).

FIG. 19 describes a comparison of Nle-TPRSFL (SEQ ID NO: 15) branch PEG with new CatK substrate with backbone Peg6. A) CatK (backbone peg6). B) Nle-TPRSFL (SEQ ID NO: 15) (Original branched).

FIG. 20A, FIG. 20B and FIG. 20C describe Synthesis if MMP selective ACPPs in Cy5Cy7 format. (Cy7)-NH2-e9-c(Peg12)-0-Substrate-r9-c (Cy5)-CONH2. FIG. 20A Control. FIG. 20B MMP2 selective TIAHLA (SEQ ID NO: 25). FIG. 20C MMP9 selective SNPYGY (SEQ ID NO: 23).

FIG. 21 describes cleavage of derivatives of RS-(Cit)-G-(homoF)-YLY (SEQ ID NO: 4) cut with MMP-2, MMP-9 and MT1-MMP.

FIG. 22 describes cleavage of derivatives of RS-(Cit)-G-(homoF)-YLY (SEQ ID NO: 4) cut with MMP-2, MMP-9 and MT1-MMP. Insertion of Proline at P3/P4 site makes the substrate a good MMP2 substrate.

FIG. 23 describes cleavage of derivatives of RS-(Cit)-G-(homoF)-YLY (SEQ ID NO: 4) cut with MT2-MMP, Only RS-Q-G-(homoF)-YLY (SEQ ID NO: 71) shows significant cleavage by MT2-MMP.

FIG. 24 describes cleavage of derivatives of RS-(Cit)-G-(homoF)-YLY (SEQ ID NO: 4) cut with MT2-MMP, Only RS-Q-G-(homoF)-YLY (SEQ ID NO: 71) shows significant cleavage by MT2-MMP.

FIG. 25 provides a diagram regarding the substitution of consensus amino acids to our current best optimal MMT1 cleavable substrate.

FIG. 26 describes MMP2/9/14 cleavage of FAM-e9-dPEG(6)-SG-XXXXXX-TA-r9-(D-cys)-NH2 peptides.

FIG. 27 describes digestion of new ACPP with MMP2, 9 and 14 from Ratinakov et. al. (2 hours/2 uM peptide/50 nM enzyme. FAM-e9-dPEG(6)-SG-XXXXXX-TA-r9-(D-cys)-NH2.

FIG. 28A, FIG. 28B, FIG. 28C and FIG. 28D describe generation of new FRET (FAM/Cy5) versions of MT1-MMP(MMP-14) selective ACPPs. FIG. 28A 0-RSHP(Hfe)TLY-(SEQ ID NO: 19) substrate. FIG. 28B O-RSHG(Hfe)FLY (SEQ ID NO: 20) substrate. FIG. 28C Original-R-S-cit-G-Hfe-YLY (SEQ ID NO: 38) substrate. FIG. 28D dPEG6-SG-ARGIKL-TA (SEQ ID NO: 37) substrate.

FIG. 29A, FIG. 29B, FIG. 29C and FIG. 29D describe FIG. 29A Higher MMP expression in tumors versus normal tissue in TCGA HNSCC. FIG. 29B HPV+ tumors have lower MMP expression than HPV-tumors. FIG. 29C and FIG. 29D Higher MMP-2/MMP-14 expression in HPV+ tumors correlates with poorer prognosis.

FIG. 30 describes RACPP schematic showing (A) no tumor-contrast immediately post-injection; (B) high tumor-contrast following MMP-dependent cleavage, separating Cy5 from Cy7. (C) Application of RACPP to HNSCC specimens produces faster Cy5/Cy7 ratio-change compared to normal tissue.

FIG. 31 describes (A,B) RACPP injection produces greater ratiometric fluorescent signal in HNSCC tumor vs. normal tissue. (C) Uncleavable-control does not produce tumor-specific contrast. (D) RACPP is sensitive and specific for tumor detection. E) Receiver operating characteristic analysis.

FIG. 32 describes (A)Ratiometric images showing higher fluorescence in tumor (white stippling). (B) Corresponding H&E images confirming tumor burden (red stippling). (C) RACPP uptake correlates directly with tumor burden.

FIG. 33 describes TCGA data showing that for patients with HPV+ tumor, mRNA expression of MMP-2 and MMP-14 positively correlate.

FIG. 34 describes mouse HNSCC tongue xenografts demonstrate greater MMP2/9 activity compared to normal tongue tissue. Bar graphs display MMP activity of samples as a percentage of activity of pure MMP standard.

FIG. 35 describes the ratio of tumor:control tissue MMP expression levels (red means high ratio, blue means low ratio) for multiple cancers represented within TCGA. HNSCC is the first column.

FIG. 36 provides a graph comparing uPA(aka PLAU) mRNA expression levels in TCGA specimens showing increased levels in tumor (red) compared to paired normal tissue (blue) in multiple cancers including HNSC (number of specimen pairs analyzed for a given tumor site in parentheses, p<0.01 for all tumor types shown).

FIG. 37 describes a) Schematics of regular non-ratiometric ACPP (Standard ACPP) and RACPP induced tumor contrast shown in top and bottom panels respectively. Immediately after IV injection neither configurations produce any tumor contrasts (left panels). Within 1-2 hr spectacular tumor contrast can be obtained with RACPP (bottom middle pane). However poor pharmacokinetic washout of the uncleaved probe with the standard ACPP results in modest tumor to background contrast (top middle panel). Longer waiting time such as 24 hr after IV injections result in loss of tumor contrasts in either configurations (right panels). b) Graph shows the emission spectrum of RACPP1, measured in mouse plasma in a cuvet spectrofluorometer, before (black solid curve) and after (red dashed curve) treatment with MMP-9. The starting spectrum shows considerable quenching of the Cy5 peak at 670 nm and re-emission from Cy7 at 780 nm.

FIG. 38 provides a schematic of RACPPs demonstrating the modular nature of the molecule which enables rational modification of the cleavable site (green) as well as payloads (yellow circles).

FIG. 39 describes that a ACPP fluorescence can be used to guide ex vivo examination of surgical specimens. Photomicrographs showing a representative specimens from tumor bearing mice following IV administration of ACPPD. (A) Low-power Cy5 fluorescence showing positive ACPPD uptake (arrowheads). (B) The same section as in A stained with H&E, confirming the presence of malignant cells in regions that show increased fluorescence uptake (arrowheads). (C and E) Enlarged fluorescence images from the boxed areas in A, showing the demarcation between high (*) and low (arrows) fluorescence uptake. (D and F) Histological (H and E) analysis of C and E, showing that the areas of high fluorescence uptake correspond to malignant cells (*). (Scale bar in A and B: 0.5 mm; C and D: 0.1 mm; E and F: 0.25 mm.) Adapted from Nguyen et al 2010.

FIG. 40 shows application of PLGCMeAG-RACPP (SEQ ID NO: 2) to HNSCC specimens produces faster Cy5/Cy7 ratio-change compared to normal tissue. Adapted from Hauff et al 2014.

FIG. 41 shows an increase in Cy5/Cy7 signal ratio of substrates YGRAAA (SEQ ID NO: 17) upone cleavage by uPA (light purple) compared to MMPs. Whitney et al, manuscript in preparation.

FIG. 42 shows a ratiometric fluorescence RACPP uptake (A) correlates with H&E evidence of tumor burden (B) from Hauff et al, 2014).

FIG. 43 shows a ratiometric fluorescence RACPP uptake (A-C) correlates with more aggressive tumor genotype (D) (from Raju et al, 2015).

### DETAILED DESCRIPTION OF THE INVENTION

The present invention is based in part on the discovery that ex vivo cleavage of ratiometric MTSs (ACPPs) by tumor extract correlates with in-vivo MTS (ACPP) fluorescence uptake and increased emission ratio in cancer, particularly carcinoma. In some embodiments, measuring the ability of individual tumors to cleave MTSs (ACPPs) and assessing the percentage of enzymatically positive tumors in a clinical population provides valuable data in that the ex vivo cleavage data can be correlated with MTS (ACPP) performance in vivo. In some embodiments, the ex vivo cleavage assay may be further developed into a personalized screening assay to determine eligibility to use MTSs (ACPPs) during a given patient procedure such as for example surgery. In some embodiments, the present invention provides methods for assessing the distribution of human surgical specimens with respect to their ability to cleave the MTSs (ACPPs) and the correlation of the MTS with clinical grade and outcome. Methods and compositions useful in such methods are provided below.

### CERTAIN DEFINITIONS

The following terms have the meanings ascribed to them unless specified otherwise.

The terms cell penetrating peptide (CPP), activatable cell penetrating peptide (ACPP), membrane translocating sequence (MTS) and protein transduction domain are used interchangeably. As used herein, the terms mean a peptide (polypeptide or protein) sequence that is able to translocate across the plasma membrane of a cell. In some embodiments, a CPP facilitates the translocation of an extracellular molecule across the plasma membrane of a cell. In some embodiments, the CPP translocates across the plasma membrane by direct penetration of the plasma membrane, endocytosis-mediated entry, or the formation of a transitory structure. In some embodiments the MTS is not transported across the membrane of a cell, but is employed in an ex vivo assay or application.

As used herein, the term “aptamer” refers to a DNA or RNA molecule that has been selected from random pools based on their ability to bind other molecules with high affinity specificity based on non-Watson and Crick interactions with the target molecule (see, e.g., Cox and Ellington, Bioorg. Med. Chem. 9:2525-2531 (2001); Lee et al., Nuc. Acids Res. 32:D95-D100 (2004)). In some embodiments, the aptamer binds nucleic acids, proteins, small organic compounds, vitamins, inorganic compounds, cells, and even entire organisms.

The terms “polypeptide,” “peptide” and “protein” and derivatives thereof as used herein, are used interchangeably herein to refer to a polymer of amino acid residues. The terms apply to naturally occurring amino acid polymers as well as amino acid polymers in which one or more amino acid residues is a non-naturally occurring amino acid (e.g., an amino acid analog). The terms encompass amino acid chains of any length, including full length proteins (i.e., antigens), wherein the amino acid residues are linked by covalent peptide bonds. As used herein, the terms “peptide” refers to a polymer of amino acid residues typically ranging in length from 2 to about 50 residues. In certain embodiments the peptide ranges in length from about 2, 3, 4, 5, 7, 9, 10, or 11 residues to about 50, 45, 40, 45, 30, 25, 20, or 15 residues. In certain embodiments the peptide ranges in length from about 8, 9, 10, 11, or 12 residues to about 15, 20 or 25 residues. Where an amino acid sequence is provided herein, L-, D-, or beta amino acid versions of the sequence are also contemplated as well as retro, inversion, and retro-inversion isoforms. Peptides also include amino acid polymers in which one or more amino acid residues is an artificial chemical analogue of a corresponding naturally occurring amino acid, as well as to naturally occurring amino acid polymers. In addition, the term applies to amino acids joined by a peptide linkage or by other modified linkages (e.g., where the peptide bond is replaced by an α-ester, a β-ester, a thioamide, phosphonamide, carbamate, hydroxylate, and the like (see, e.g., Spatola, Chem. Biochem. Amino Acids and Proteins 7: 267-357 (1983)), where the amide is replaced with a saturated amine (see, e.g., Skiles et al., U.S. Pat. No. 4,496,542, which is incorporated herein by reference, and Kaltenbronn et al., (1990) Pp. 969-970 in Proc. 11th American Peptide Symposium, ESCOM Science Publishers, The Netherlands, and the like)).

The term “amino acid” and derivatives thereof as used herein, refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. Amino acids may be either D amino acids or L amino acids. In peptide sequences throughout the specification, lower case letters indicate the D isomer of the amino acid (conversely, upper case letters indicate the L isomer of the amino acid).

Amino acids may be referred to herein by either their commonly known three letter symbols or by the one-letter symbols recommended by the IUPAC-IUB Biochemical Nomenclature Commission.

Nucleotides, likewise, may be referred to by their commonly accepted single-letter codes.

One of skill will recognize that individual substitutions, deletions or additions to a peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

As used herein, a “linker” is any molecule capable of binding (e.g., covalently) portion A and portion B of a MTS molecule disclosed herein. Linkers include, but are not limited to, straight or branched chain carbon linkers, heterocyclic carbon linkers, peptide linkers, and polyether linkers. For example, poly(ethylene glycol) linkers are available from Quanta Biodesign, Powell, Ohio. These linkers optionally have amide linkages, sulfhydryl linkages, or heterofunctional linkages.

As used herein, the term “label” refers to any molecule that facilitates the visualization and/or detection of a MTS molecule disclosed herein. In some embodiments, the label is a fluorescent moiety.

The term “carrier” means an inert molecule that increases (a) plasma half-life and (b) solubility. In some embodiments, a carrier increases plasma half-life and solubility by reducing glomerular filtration. In some embodiments, a carrier increases tumor uptake due to enhanced permeability and retention (EPR) of tumor vasculature.

The term “thrombin” means an enzyme (EC 3.4.21.5) that cleaves fibrinogen molecules into fibrin monomers. Thrombin, acting through its G-protein coupled receptor PAR-I, is a key player in a wide range of vascular and extravascular disease processes throughout the body, including cancer, cardiovascular diseases, acute kidney injury, and stroke. In certain instances, thrombin activity increases over the course of atherosclerotic plaque development. In some embodiments, thrombin activity is a biomarker for atherosclerotic plaque development.

The terms “individual,” “patient,” or “subject” are used interchangeably. As used herein, they mean any mammal (i.e. species of any orders, families, and genus within the taxonomic classification animalia: chordata: vertebrata: mammalia). In some embodiments, the mammal is a human. None of the terms require or are limited to situation characterized by the supervision (e g constant or intermittent) of a health care worker (e g a doctor, a registered nurse, a nurse practitioner, a physician's assistant, an orderly, or a hospice worker).

As used herein, the term “medical professional” means any health care worker. By way of non-limiting example, the health care worker may be a doctor, a registered nurse, a nurse practitioner, a physician's assistant, an orderly, or a hospice worker.

The terms “administer,” “administering,” “administration,” and derivatives thereof as used herein, refer to the methods that may be used to enable delivery of agents or compositions to the desired site of biological action These methods include, but are not limited to parenteral injection (e g, intravenous, subcutaneous, intraperitoneal, intramuscular, intravascular, intrathecal, intravitreal, infusion, or local) Administration techniques that are optionally employed with the agents and methods described herein, include e g, as discussed in Goodman and Gilman, The Pharmacological Basis of Therapeutics, current ed, Pergamon, and Remington's, Pharmaceutical Sciences (current edition), Mack Publishing Co, Easton, Pa.

The term “pharmaceutically acceptable” and derivatives thereof as used herein, refers to a material that does not abrogate the biological activity or properties of the agents described herein, and is relatively nontoxic (i e, the toxicity of the material significantly outweighs the benefit of the material) In some instances, a pharmaceutically acceptable material may be administered to an individual without causing significant undesirable biological effects or significantly interacting in a deleterious manner with any of the components of the composition in which it is contained.

The term “surgery” and derivatives thereof as used herein, refers to any methods for that may be used to manipulate, change, or cause an effect by a physical intervention These methods include, but are not limited to open surgery, endoscopic surgery, laparoscopic surgery, minimally invasive surgery, and robotic surgery.

The terms “neoplasm” or “neoplasia” and derivatives thereof as used herein, include any non-normal or non-standard cellular growth. Neoplasms can include tumors and cancers of any variety of stages, from benign to metastatic. Neoplasms can be primary or metastatic growths and can occur anywhere in a subject. Neoplasms can include neoplasms of the lung, skin, lymph, brain, nerves, muscle, breast, prostate, testis, pancreases, liver, kidneys, stomach, muscle, bone and blood. Neoplasms can be solid and non-solid tumors.

The terms “sample” or “samples” and derivatives thereof as used herein, include any samples obtained from a subject with can be employed with the methods described herein. Samples can include but are not limited to urine, blood, lymph, tears, mucus, saliva, biopsy or other sample tissue samples. Sample can be frozen, refrigerated, previously frozen, and/or stored for minutes, hours, days, weeks, months, years. Sampling techniques, handling and storage are well known and any such techniques for obtaining samples for use with the present invention are contemplated.

The following symbols, where used, are used with the indicated meanings Fl=fluorescein, aca=ahx=X=ammohexanoyl linker (—HN—(CH2)<rCO-)aminohexanoyl, C=L-cysteine, E=L-glutamate, R=L-arginme, D=L-aspartate, K=L-lysine, A=L-alanine, r=D-arginine, c=D-cysteine, e=D-glutamate, P=L-proline, L=L-leucine, G=glycine, V=valine, I=isoleucine, M=methionine, F==phenylalanine, Y=tyrosine, W=tryptophan, H=histidine, Q=glutamine, N=asparagine, S=serine, T=threonine, o is 5-amino-3-oxapentanoyl linker, and C(me) is S-methylcysteine.

### Methods of Use

The MTS molecules find use in a variety of ex vivo applications as described herein and such MTS molecules have been thoroughly described (see, WO 2005/042034, WO/2006/125134, WO2011008992 and WO2011008996; all of which are incorporated herein by reference in their entireties). As such, according to disclosure contained herein, this invention pertains to methods and compositions that find use in diagnostic, prognostic (e.g., patient prognosis) and characterization (e.g., histologic grade/stage) of neoplasm samples based on the ability of a tumor sample to cleave a MTS molecule of the present invention.

Methods of use and compositions comprising MTS molecules are disclosed. Molecules having features of the invention include peptide portions linked by a cleavable linker portion which may be a peptide. The inventors have found that these MTS molecules can find use in diagnostic, detection, screening, prognosis (e.g., patient prognosis) and characterization (e.g., histologic grade/stage) assays.

According to the present invention, such methods are based in part on cleavage of the MTS molecule and detection of that cleavage event. The presence of one or more proteases in a sample from a subject can be detected ex vivo based on cleavage of the peptide. Such cleavage is detected by detecting a change in a detectable label (detectable moiety) that is part of the MTS peptide. In some embodiments, the MTS molecule contains a detectable moieties which provide for an indication of a cleavage event. In some embodiments, cleavage could be detected by size changes in the length of the peptide (e.g., gel electrophoresis, size exclusion, column chromatography, immunoflourescence, etc.) or other biochemical and physical changes that occur to the MTS molecule. In some embodiments, the MTS molecule comprises a label which facilitates cleavage detection. In some embodiments, cleavage could be detected using a FRET-based pair (a reporter dye and an acceptor dye that are involved in fluorescence resonance energy transfer known as FRET), where a change in fluorescence is indicative of a cleavage event. See, for examples, Examples 1-3. Methods for detecting and monitoring cleavage of proteins are well known and any such methods could be employed in detecting cleavage of the MTS molecules of the invention.

In some embodiments, the invention provides an ex vivo method for detecting the presence of one or more protease activities in a neoplasia sample comprising a) combining ex vivo said sample from a subject with a molecule of the structure A-X-B-C, wherein cleavage of said A-X-B-C is indicative of the presence of protease activity and wherein B is a peptide portion of about 5 to about 20 basic amino acid residues, which is suitable for cellular uptake, A is a peptide portion of about 2 to about 20 acidic amino acid residues, which when linked with portion B is effective to inhibit or prevent cellular uptake of portion B, and X is a cleavable linker of about 2 to about 100 atoms joining A with B, where X is cleavable under physiological conditions, and C is a detectable moiety; and b) detecting cleavage of A-X-B-C by detecting a change in said detectable moiety C, wherein said change in C is indicative of cleavage and said cleavage is indicative of the presence of one or more protease activities in said neoplasia. In some embodiments, one protease activity can be detected. In some embodiments, 2, 3, 4, 5, 6, 7, 8, 9 or 10 protease activities can be detected. In some embodiments, one or more protease activities can be detected.

In some embodiments, the invention provides an ex vivo method of screening for the presence of one or more protease activities in a neoplasia sample comprising combining ex vivo said neoplasia sample from a subject with a molecule of the structure A-X-B-C, wherein B is a peptide portion of about 5 to about 20 basic amino acid residues, which is suitable for cellular uptake, A is a peptide portion of about 2 to about 20 acidic amino acid residues, which when linked with portion B is effective to inhibit or prevent cellular uptake of portion B, and X is a cleavable linker of about 2 to about 100 atoms joining A with B, where X is cleavable under physiological conditions, and C is a detectable moiety; and b) detecting cleavage of A-X-B-C by detecting a change in said detectable moiety C, wherein said change in C is indicative of cleavage and said cleavage is indicative of the presence of one or more protease activities in said neoplasia. In some embodiments the MTS molecules can be used in screening assays to determine how many proteases and/or which proteases are expressed by a sample. In some embodiments, the screening is small scale, involving screening of 1, 5, 10, 20 or 30 samples. In some embodiments, screening is large scale, and involves screening of 100, 500, 1000, 10000, 100 000, 500000 or more samples. In some embodiments, samples are screened for 1, 2, 3, 4, 5, 6, 7, 8, 9, 10 or more protease activities using MTS molecules of the invention. In some embodiments, screening information can be employed to develop data bases and incorporated with other bioinformatic information in order to develop protease profiles of samples.

In some embodiments, the invention provides an ex vivo method of determining the protease profile of a neoplasia sample, comprising a) combining said sample from a subject with a molecule of the structure A-X-B-C, wherein B is a peptide portion of about 5 to about 20 basic amino acid residues, which is suitable for cellular uptake, A is a peptide portion of about 2 to about 20 acidic amino acid residues, which when linked with portion B is effective to inhibit or prevent cellular uptake of portion B, and X is a cleavable linker of about 2 to about 100 atoms joining A with B, where X is cleavable under physiological conditions and C is a detectable moiety; and b) detecting cleavage of A-X-B-C by detecting a change in said detectable moiety C, wherein said change in C is indicative of cleavage and said cleavage is indicative of the presence of one or more protease activities in said neoplasia and wherein the protease profile is developed based on the cleavage detected. In some embodiments, the MTS molecules are employed to develop a protease profile for one or more neoplasia samples. Protease profiles can be employed to develop databases and can be incorporated with other information, including for example bioinformatic information, in order to develop protease profiles of neoplasia samples and for protease profiles for patients with neoplasia.

In some embodiments, the invention provides An ex vivo method of determining a treatment regimen based on the protease profile of a neoplasia sample, comprising a) combining ex vivo said neoplasia sample from a subject with a molecule of the structure A-X-B-C, wherein B is a peptide portion of about 5 to about 20 basic amino acid residues, which is suitable for cellular uptake, A is a peptide portion of about 2 to about 20 acidic amino acid residues, which when linked with portion B is effective to inhibit or prevent cellular uptake of portion B, and X is a cleavable linker of about 2 to about 100 atoms joining A with B, where X is cleavable under physiological conditions and C is a detectable moiety; and b) detecting cleavage of A-X-B-C by detecting a change in detectable moiety C, wherein said change in C is indicative of cleavage and said cleavage is indicative of the presence of one or more protease activities and wherein the presence and/or absence of one or more protease activities allows for determining a medical treatment regimen. In some embodiments, the MTS molecules are employed to determine a treatment regimen. Protease information and/or protease profiles can employed to develop databases and can be incorporated with other information, for example bioinformatic information, in order to develop protease profiles of samples. In some embodiments, such information can be combined with information regarding treatment and surgical options know to those of skill in the medical arts in order to determine and develop personalize treatment regimens for individual subjects. In some embodiments, the medical regimen is a surgical regimen. After detecting the presence or absence of one or more proteases based on MTS molecule cleavage, a determination of the usefulness of an MTS molecule in surgical procedures can be determined. Detection of cleavage of the MTS molecule would be indicative of the presence of one or more proteases and such information would allow for a determination of usefulness of the peptide in a surgical procedure in order to detect tumor borders and assist with surgical removal as previously described (See, e.g., see, WO 2005/042034, WO/2006/125134, WO2011008992 and WO2011008996). Non-detection of cleavage of the MTS molecule would be indicative of the absence of a protease and the non-usefulness of the peptide in a surgical procedure.

In some embodiments, the invention provides an ex vivo method of characterizing a neoplasia based on the protease profile of said neoplasia, comprising a) combining a sample of said neoplasia from a subject with a molecule of the structure A-X-B-C, wherein B is a peptide portion of about 5 to about 20 basic amino acid residues, which is suitable for cellular uptake, A is a peptide portion of about 2 to about 20 acidic amino acid residues, which when linked with portion B is effective to inhibit or prevent cellular uptake of portion B, and X is a cleavable linker of about 2 to about 100 atoms joining A with B, where X is cleavable under physiological conditions and C is a detectable moiety; and detecting cleavage of A-X-B-C by detecting a change is said detectable moiety C, wherein said change in C is indicative of cleavage and said cleavage is indicative of the presence of more than one protease activities and wherein the characterization of the neoplasia is based on the cleavage detected. In some embodiments, the neoplasia is characterized based on histology, stage, grade, location, type, or any of a variety of characteristics known to those skilled in the medical arts. In some embodiments, the protease profile is correlated with histology, stage, grade, location, type, or any of a variety of characteristics known to those skilled in the medical arts in order to characterize the neoplasia. In some embodiments, the presence of the protease activity is indicative of neoplasia. In some embodiments, the presence of the protease activity is indicative of metastasis.

In some embodiments, the present invention provides a diagnostic composition for use in the methods of any of the preceding claims comprising: a molecule of the structure A-X-B-C, wherein B is a peptide portion of about 5 to about 20 basic amino acid residues, which is suitable for cellular uptake, A is a peptide portion of about 2 to about 20 acidic amino acid residues, which when linked with portion B is effective to inhibit or prevent cellular uptake of portion B, and X is a cleavable linker of about 3 to about 30 atoms joining A with B, where X is a cleavable under physiological conditions and C is a detectable moiety; and a diagnostic buffering agent. In some embodiments of the diagnostic composition, the cleavable linker X is of between about 6 to about 30 atoms in length, said portion A has between about 5 to about 9 acidic amino acid residues, and said portion B has between about 9 to about 16 basic amino acid residues.

In some embodiments, the present invention provides an array comprising: a plurality of molecules of the structure A-X-B-C, wherein B is a peptide portion of about 5 to about 20 basic amino acid residues, which is suitable for cellular uptake, A is a peptide portion of about 2 to about 20 acidic amino acid residues, which when linked with portion B is effective to inhibit or prevent cellular uptake of portion B, and X is a cleavable linker of about 3 to about 30 atoms joining A with B, where X is a cleavable under physiological conditions, and C is a detectable moiety. In some embodiments of the array, the cleavable linker X is of between about 6 to about 30 atoms in length, said portion A has between about 5 to about 9 acidic amino acid residues, and said portion B has between about 9 to about 16 basic amino acid residues. In some embodiments, the array comprises a plurality of molecules of the structure A-X-B and wherein the cleavable linker X comprises a plurality of cleavable linkers X. In some embodiments of the array, the plurality of cleavable linkers X linking a portion A to a structure B-C are cleavable by a single protease. In some embodiments of the array, the plurality of cleavable linkers X linking a portion A to a structure B-C are cleavable by more than one protease. In some embodiments, an array of the invention would contain a plurality of one species (one type) of MTS molecules. In some embodiments, an array of the invention would contain a plurality of one species (one type) of MTS molecules and multiple samples could be screened for one protease activity type. In some embodiments, an array of the invention would contain a plurality of a plurality of species (multiple types) of MTS molecules. In some embodiments, an array of the invention would contain a plurality of a plurality of species (multiple types) of MTS molecules and one or more samples could be screened for one or more protease activity types. An array can include but is not limited to any substrate to which the MTS molecules can be bound, and can include for examples solid substrates, micro-arrays and microchips. Methods for making arrays are well known and can even be supplied by commercial suppliers. Arrays can be manually processed and/or automated or a combination thereof. Such arrays can be employed in low-throughput as well as high-throughput applications and can analyze one or more samples, one or more proteases or any combination thereof.

In some embodiments of the above described methods, ratiometric analysis can be employed to determine the level of enzyme activity and/or to assess the percentage of enzymatically positive tumors in a population. Such ratiometric analyses can be based on the ratio of cleaved to non-cleaved MTS molecules. In some embodiments, ratiometric analysis can be employed to correlate ex vivo cleavage with in vivo cleavage activities.

In some embodiments, the protease information can be correlated with histology, grade, type, characterization, etc. in order to better characterize neoplasias and to provide personalized prognosis and treatment regimens. Such information can be provided to those of skill in the medical arts and be employed to develop personalized medical treatment regimens for individuals.

### MTS Peptides

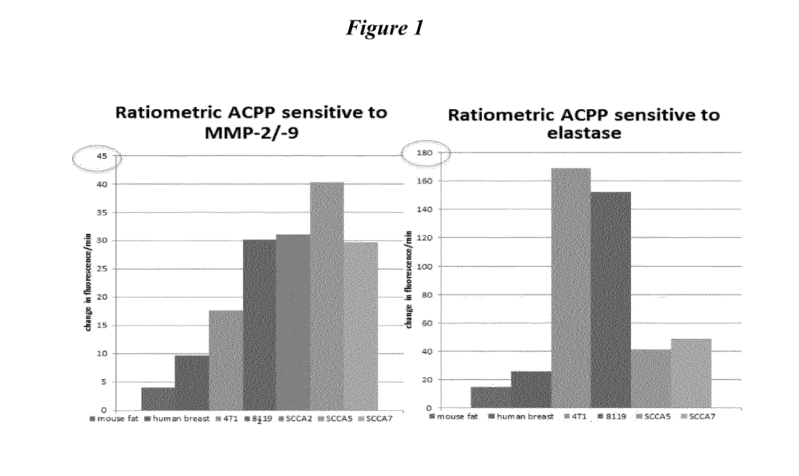

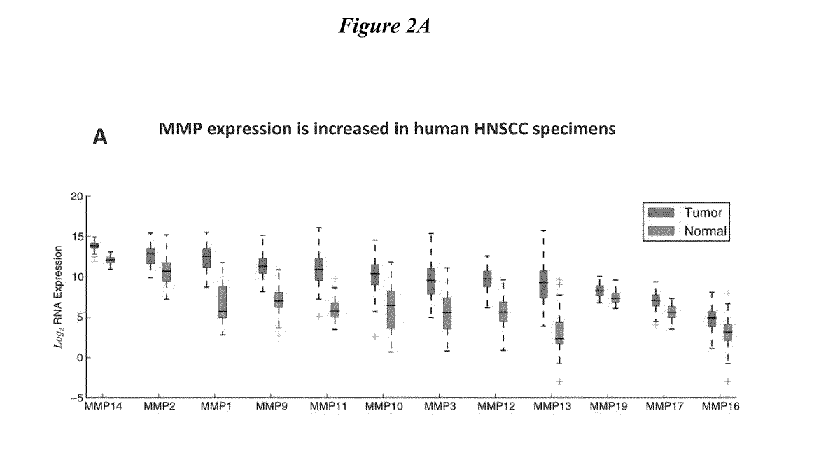

In one embodiment, a generic structure for peptides having features of the invention is A-X-B, where peptide portion B includes between about 5 to about 20 basic amino acids, X is a cleavable linker portion, in some embodiments cleavable under physiological conditions, and where peptide portion A includes between about 2 to about 20 acidic amino acids. In some embodiments of molecules having features of the invention, peptide portion B includes between about 5 to about 20, or between about 9 to about 16 basic amino acids, and may be a series of basic amino acids (e.g., arginines, histidines, lysines, or other basic amino acids). In some embodiments of molecules having features of the invention, peptide portion A includes between about 2 to about 20, or between about 5 to about 20 acidic amino acids, and may be series of acidic amino acids (e.g., glutamates and aspartates or other acidic amino acids). A schematic representation of a MTS molecule having features of the invention comprising a basic portion B, a linker portion X, and an acidic portion A is presented in FIG. 1A of WO 2005/042034. In embodiments, MTS molecules having features of the invention may be cyclic molecules, as schematically illustrated in FIG. 1B of WO 2005/04203. Thus, MTS molecules having features of the invention may be linear molecules, cyclic molecules, or may be linear molecules including a cyclic portion.

In some embodiments, a MTS molecule disclosed herein has the formula A-X-B-C, wherein C is a cargo moiety (including for example a detectable moiety); A is a peptide with a sequence comprising 5 to 9 consecutive acidic amino acids, wherein the amino acids are selected from: aspartates and glutamates; B is a peptide with a sequence comprising 5 to 20 consecutive basic amino acids; and X is a linker that is cleavable by protease.

In some embodiments, a MTS molecule disclosed herein has the formula (A-X-B-Qn-M, wherein C is a cargo moiety; A is a peptide with a sequence comprising 5 to 9 consecutive acidic amino acids, wherein the amino acids are selected from: aspartates and glutamates; B is a peptide with a sequence comprising 5 to 20 consecutive basic amino acids; X is a linker that is cleavable by thrombin; and M is a macromolecular carrier.

Regulation of transport into and out of a cell is important for its continued viability. For example, cell membranes contain ion channels, pumps, and exchangers capable of facilitating the transmembrane passage of many important substances. However, transmembrane transport is selective in addition to facilitating the entry of desired substances into a cell, and facilitating the exit of others, a major role of a cell membrane is to prevent uncontrolled entry of substances into the cell interior. This barrier function of the cell membrane makes difficult the delivery of markers, drugs, nucleic acids, and other exogenous material into cells.

As discussed above, molecules including a multiple basic amino acids, such as a series of basic amino acids, are often taken up by cells. However, the present inventors have discovered that molecules having structures including a basic portion B, a linker portion X, and an acidic portion A are not taken up by cells. An acidic portion A may include amino acids that are not acidic. Acidic portion A may comprise other moieties, such as negatively charged moieties. In embodiments of MTS molecules having features of the invention, an acidic portion A may be a negatively charged portion, in some embodiments having about 2 to about 20 negative charges at physiological pH, that does not include an amino acid. A basic portion B may include amino acids that are not basic. Basic portion B may comprise other moieties, such as positively charged moieties. In embodiments of MTS molecules having features of the invention, a basic portion B may be a positively charged portion, having between about 5 and about 20 positive charges at physiological pH, that does not include an amino acid. Including an acidic portion A is effective to inhibit or prevent the uptake of a portion B into cells. Such a block of uptake that would otherwise be effected by the basic amino acids of portion B may be termed a “veto” of the uptake by the acidic portion A. The present inventors have made the further surprising discovery that cleavage of linker X, allowing the separation of portion A from portion B is effective to allow the uptake of portion B into cells.

In a further embodiment, a generic structure for peptides having features of the invention is A-X-B-C, where C is a cargo moiety, X a linker, A an acidic portion, and B a basic portion. An acidic portion A may include amino acids that are not acidic. Acidic portion A may comprise other moieties, such as negatively charged moieties. In embodiments of MTS molecules having features of the invention, an acidic portion A may be a negatively charged portion, having about 2 to about 20 negative charges at physiological pH, that does not include an amino acid. A basic portion B may include amino acids that are not basic. Basic portion B may comprise other moieties, such as positively charged moieties. In embodiments of MTS molecules having features of the invention, a basic portion B may be a positively charged portion, having between about 5 and about 20 positive charges at physiological pH, that does not include an amino acid. In some embodiments, the amount of negative charge in portion A is approximately the same as the amount of positive charge in portion B.

A cargo moiety C may be, for example, a variety of detectable agents, including for example any detectable moiety for detection in an ex vivo assay, a contrast agent for diagnostic imaging, or a chemotherapeutic drug or radiation-sensitizer for therapy. B may be, for example, a peptide portion having between about 5 to about 20 basic amino acids, such as a series of basic amino acids (arginines are can be employed, as well as histidines, lysines or other basic amino acids). In some embodiments, X is a cleavable linker that is cleavable under physiological conditions. A may be a peptide portion having between about 2 to about 20 about 2 to about 20 acidic amino acids, such as a series of acidic amino acids. In some embodiments of molecules having features of the invention, glutamates and aspartates are employed as acidic amino acids for peptide portion A.

The present inventors have made the surprising discovery that including an acidic portion A is also effective to inhibit or prevent the uptake into cells of molecules combining a portion B and a portion C. The present inventors have made the further discovery that cleavage of linker X, allowing the separation of portion A from portion B is effective to allow the uptake of portions B and C into cells. Thus, delivery of cargo C can be controlled and enhanced by molecules having features of the invention.

For example, when peptide portion A contains about 5 to about 9 consecutive glutamates or aspartates, and X is a flexible linker of about 2 to about 100, or about 6 to about 30 atoms in length, the normal ability of a peptide portion B (e.g., a sequence of nine consecutive arginine residues) to cause uptake into cells is blocked. Cleavage of linker X allows the separation of portion A from portion B and portion C, alleviating the veto by portion A. Thus, when separated from A, the normal ability of portion B to effect the uptake of cargo C into cells is regained. Such cellular uptake typically occurs near the location of the cleavage event. Thus, design of cleavable linker X such that it is cleaved at or near a target cell is effective to direct uptake of cargo C into target cells. Extracellular cleavage of X allows separation of A from the rest of the molecule to allow uptake into cells.

A MTS molecule having features of the invention may be of any length. In embodiments of MTS molecules having features of the invention, a MTS molecule may be about 7 to about 40 amino acids in length, not including the length of a linker X and a cargo portion C. In other embodiments, particularly where multiple non-acidic (in portion A) or non-basic (in portion B) amino acids are included in one or both of portions A and B, portions A and B of a MTS molecule may together be about 50, or about 60, or about 70 amino acids in length. A cyclic portion of an MTS may include about 12 to about 60 amino acids, not including the length of a linker X and a cargo portion C. For example, a linear MTS molecule having features of the invention may have a basic portion B having between about 5 to about 20 basic amino acids (in some embodiments between about 9 to about 16 basic amino acids) and an acidic portion A having between about 2 to about 20 acidic amino acids (e.g., between about 5 to about 20, between about 5 to about 9 acidic amino acids). In some embodiments, a MTS molecule having features of the invention may have a basic portion B having between about 9 to about 16 basic amino acids and between about 5 to about 9 acidic amino acids.

Portions A and B may include either L-amino acids or D-amino acids. In embodiments of the invention, D-amino acids are employed for the A and B portions in order to minimize immunogenicity and nonspecific cleavage by background peptidases or proteases. Cellular uptake of oligo-D-arginine sequences is known to be as good or better than that of oligo-L-arginines. The generic structures A-X-B and -A-X-B C can be effective where A is at the amino terminus and where A is at the carboxy terminus, i.e. either orientation of the peptide bonds is permissible. However, in embodiments where X is a peptide cleavable by a protease, it may be beneficial to join the C-terminus of X to the N-terminus of B, so that the new amino terminus created by cleavage of X contributes an additional positive charge that adds to the positive charges already present in B.

In some embodiments, a MTS molecule disclosed herein has the formula A-X-B-C, wherein C is a cargo moiety; A is a peptide with a sequence comprising 5 to 9 consecutive acidic amino acids, wherein the amino acids are selected from: aspartates and glutamates; B is a peptide with a sequence comprising 5 to 20 consecutive basic amino acids; and X is a linker that is cleavable by thrombin. In some embodiments, the acid amino acids are consecutive. In some embodiments, the acid amino acids are not consecutive.

In some embodiments, a MTS molecule disclosed herein has the formula (A-X-B-C)n-M, wherein C is a cargo moiety; A is a peptide with a sequence comprising 5 to 9 consecutive acidic amino acids, wherein the amino acids are selected from: aspartates and glutamates; B is a peptide with a sequence comprising 5 to 20 consecutive basic amino acids; X is a linker that is cleavable by thrombin; M is a macromolecular carrier; and n is an integer between 1 and 20.

In some embodiments, a MTS molecule disclosed herein has the formula (A-X-B)n-D, wherein C is a cargo moiety; A is a peptide with a sequence comprising 5 to 9 consecutive acidic amino acids, wherein the amino acids are selected from: aspartates and glutamates; B is a peptide with a sequence comprising 5 to 20 consecutive basic amino acids; X is a linker that is cleavable by thrombin; D is a dendrimer; and n is an integer between 1 and 20. In some embodiments, D comprises a cargo moiety.

In some embodiments of molecules having features of the invention, peptide portion A includes between about 2 to about 20, or between about 5 to about 20 acidic amino acids, and may be series of acidic amino acids (e.g., glutamates and aspartates or other acidic amino acids). In some embodiments, A has a sequence comprising 5 to 9 consecutive glutamates. In some embodiments, portion A comprises 8 consecutive glutamates (i.e., EEEEEEEE or eeeeeeee).

An acidic portion A may include amino acids that are not acidic. Acidic portion A may comprise other moieties, such as negatively charged moieties. In embodiments of a MTS molecule disclosed herein, an acidic portion A may be a negatively charged portion, in some embodiments having about 2 to about 20 negative charges at physiological pH that does not include an amino acid. In some embodiments, the amount of negative charge in portion A is approximately the same as the amount of positive charge in portion B.

Portion A is either L-amino acids or D-amino acids. In embodiments of the invention, D-amino acids are can be employed in order to minimize immunogenicity and nonspecific cleavage by background peptidases or proteases. Cellular uptake of oligo-D-arginine sequences is known to be as good as or better than that of oligo-L-arginines.

It will be understood that portion A may include non-standard amino acids, such as, for example, hydroxylysine, desmosine, isodesmosine, or other non-standard amino acids. Portion A may include modified amino acids, including post-translationally modified amino acids such as, for example, methylated amino acids (e.g., methyl histidine, methylated forms of lysine, etc.), acetylated amino acids, amidated amino acids, formylated amino acids, hydroxylated amino acids, phosphorylated amino acids, or other modified amino acids. Portion A may also include peptide mimetic moieties, including portions linked by non-peptide bonds and amino acids linked by or to non-amino acid portions.

The generic structures A-X-B and -A-X-B-C is effective where A is at the amino terminus or where A is at the carboxy terminus, i.e., either orientation of the peptide bonds is permissible.

In some embodiments, a MTS molecule disclosed herein has the formula A-X-B-C, wherein C is a cargo moiety, A is a peptide with a sequence comprising 5 to 9 consecutive acidic amino acids, wherein the amino acids are selected from: aspartates and glutamates; B is a peptide with a sequence comprising 5 to 20 consecutive basic amino acids; and X is a linker that is cleavable by thrombin.

In some embodiments, a MTS molecule disclosed herein has the formula (A-X-B-C)n-M, wherein C is a cargo moiety; A is a peptide with a sequence comprising 5 to 9 consecutive acidic amino acids, wherein the amino acids are selected from: aspartates and glutamates; B is a peptide with a sequence comprising 5 to 20 consecutive basic amino acids; X is a linker that is cleavable by thrombin; M is a macromolecular carrier; and n is an integer between 1 and 20.

In some embodiments, a MTS molecule disclosed herein has the formula (A-X-B)n-D, wherein C is a cargo moiety, A is a peptide with a sequence comprising 5 to 9 consecutive acidic amino acids, wherein the amino acids are selected from: aspartates and glutamates; B is a peptide with a sequence comprising 5 to 20 consecutive basic amino acids; X is a linker that is cleavable by thrombin; D is a dendrimer; and n is an integer between 1 and 20. In some embodiments, D comprises a cargo moiety.

In some embodiments of molecules having features of the invention, peptide portion B includes between about 5 to about 20, or between about 9 to about 16 basic amino acids, and may be a series of basic amino acids (e.g., arginines, histidines, lysines, or other basic amino acids). In some embodiments, portion B comprises 9 consecutive arginines (i.e., RRRRRRRRR or rrrrrrrrr). In some embodiments, the basic amino acids are consecutive. In some embodiments, the basic amino acids are not consecutive.

A basic portion B may include amino acids that are not basic. Basic portion B may comprise other moieties, such as positively charged moieties. In embodiments, a basic portion B may be a positively charged portion, having between about 5 and about 20 positive charges at physiological pH, that does not include an amino acid. In some embodiments, the amount of negative charge in portion A is approximately the same as the amount of positive charge in portion B.

Portion B is either L-amino acids or D-amino acids. In embodiments of the invention, D-amino acids are employed in order to minimize immunogenicity and nonspecific cleavage by background peptidases or proteases. Cellular uptake of oligo-D-arginine sequences is known to be as good as or better than that of oligo-L-arginines.

It will be understood that portion B may include non-standard amino acids, such as, for example, hydroxylysine, desmosine, isodesmosine, or other non-standard amino acids. Portion B may include modified amino acids, including post-translationally modified amino acids such as, for example, methylated amino acids (e.g., methyl histidine, methylated forms of lysine, etc.), acetylated amino acids, amidated amino acids, formylated amino acids, hydroxylated amino acids, phosphorylated amino acids, or other modified amino acids. Portion B may also include peptide mimetic moieties, including portions linked by non-peptide bonds and amino acids linked by or to non-amino acid portions.

In embodiments where X is a peptide cleavable by a protease, it may be beneficial to join the C-terminus of X to the N-terminus of B, so that the new amino terminus created by cleavage of X contributes an additional positive charge that adds to the positive charges already present in B.

Cargo portion C may be attached to B in any location or orientation. A cargo portion C need not be located at an opposite end of portion B than a linker X. Any location of attachment of C to B is acceptable as long as that attachment remains after X is cleaved. For example, a cargo portion C may be attached to or near to an end of portion B with linker X attached to an opposite end of portion B. A cargo portion C may also be attached to or near to an end of portion B with linker X attached to or near to the same end of portion B. In some embodiments of the invention, a linker X may link to a cargo portion C which is linked to a basic portion B where a MTS molecule having features of the invention comprising a cargo portion C linked to multiple basic portions B, each of which basic portions B are linked to a linker portion X, and via the linker to an acidic portion A.

A linker X may be designed for cleavage in the presence of particular conditions or in a particular environment. In some embodiments, a linker X is cleavable under physiological conditions. Cleavage of such a linker X may, for example, be enhanced or may be effected by particular pathological signals or a particular environment related to cells in which cargo delivery is desired. The design of a linker X for cleavage by specific conditions, such as by a specific enzyme, allows the targeting of cellular uptake to a specific location where such conditions obtain. Thus, one important way that MTS molecules having features of the invention provide specific detection of specific proteases presence is by the design of the linker portion X to be cleaved by the protease. The linker portion X can be designed to be cleaved only by specific proteases or to be selective for specific proteases. After cleavage of a linker X, the portions B-C of the molecule are then a simple conjugate of B and C, in some instances retaining a relatively small, inert stub remaining from a residual portion of linker X.

A linker portion X may be cleavable by conditions found in the extracellular environment, such as acidic conditions which may be found near cancerous cells and tissues or a reducing environment, as may be found near hypoxic or ischemic cells and tissues; by proteases or other enzymes found on the surface of cells or released near cells having a condition to be treated, such as diseased, apoptotic or necrotic cells and tissues; or by other conditions or factors. An acid-labile linker may be, for example, a cis-aconitic acid linker. A linker portion X may also be cleaved extracellularly in an ex vivo reaction. Other examples of pH-sensitive linkages include acetals, ketals, activated amides such as amides of 2,3-dimethylmaleamic acid, vinyl ether, other activated ethers and esters such as enol or silyl ethers or esters, imines, iminiums, enamines, carbamates, hydrazones, and other linkages. A linker X may be an amino acid or a peptide. A peptide linker may be of any suitable length, such as, for example, about 3 to about 30, or about 6 to about 24 atoms in sequence (e.g., a linear peptide about 1 to 10 or about 2 to 8 amino acids long). A cleavable peptide linker may include an amino acid sequence recognized and cleaved by a protease, so that proteolytic action of the protease cleaves the linker X.

In some embodiments, X is a cleavable linker. In some embodiments, a linker X is designed for cleavage in the presence of particular conditions or in a particular environment In some embodiments, a linker X is cleavable under physiological conditions Cleavage of such a linker X may, for example, be enhanced or may be affected by particular pathological signals or a particular environment related to cells in which cargo delivery is desired The design of a linker X for cleavage by specific conditions, such as by a specific enzyme (e g, thrombin), allows the targeting of cellular uptake to a specific location where such conditions obtain Thus, one important way that MTS molecules provide specific targeting of cellular uptake to desired cells, tissues, or regions is by the design of the linker portion X to be cleaved by conditions near such targeted cells, tissues, or regions After cleavage of a linker X, the portions B-C of the molecule are then a simple conjugate of B and C, in some instances retaining a relatively small, inert stub remaining from a residual portion of linker X.

In some embodiments, X is cleaved by thrombin. In some embodiments, X is substantially specific for thrombin, MMPs or elastates. In some embodiments, X is cleaved by or is substantially specific for MMPs (PLGLAG (SEQ ID NO: 1) and PLGC(met)AG (SEQ ID NO: 2), RSHP(Hfe)TLY (SEQ ID NO: 19), RSHG(Hfe)FLY (SEQ ID NO: 20), SNPYK-Y (SEQ ID NO: 21), SNPKG-Y (SEQ ID NO: 22), SNPYG-Y (SEQ ID NO: 23), TLSE-LH (SEQ ID NO: 24), TIAHLA (SEQ ID NO: 25)), elastases (RLQLK(acetyl)L (SEQ ID NO: 26), plasmin and/or thrombin, cathepsin K (KLRFSKQ (SEQ ID NO: 27)). In some embodiments, the MMP 2,9 cleavable or substantially specific sequence is PLGLAG and/or PLGC(met)AG (SEQ ID NO: 2). In some embodiments, the MMP 14 cleavable or substantially specific sequences could include but are not limited to RSHP(Hfe)TLY (SEQ ID NO: 19) or RSHG(Hfe)FLY (SEQ ID NO: 20). In some embodiments, the MMP 9 cleavable or substantially specific sequences could include but are not limited to SNPYK-Y (SEQ ID NO: 21), SNPKG-Y (SEQ ID NO: 22), or SNPYG-Y (SEQ ID NO: 23). In some embodiments, the MMP 2 cleavable or substantially specific sequences could include but are not limited to TLSE-LH (SEQ ID NO: 24), TIAHLA (SEQ ID NO: 25). In some embodiments, the cathepsin K cleavable or substantially specific sequences could include but are not limited to KLRFSKQ (SEQ ID NO: 27). In some embodiments, the MMP cleavable or substantially specific sequences could include but are not limited to RS-(Cit)-G-(homoF)-YLY (SEQ ID NO: 4), CRPAHLRDSG (SEQ ID NO: 5), SLAYYTA (SEQ ID NO: 6), NISDLTAG (SEQ ID NO: 7), PPSSLRVT (SEQ ID NO; 8), SGESLSNLTA (SEQ ID NO: 9), RIGFLR (SEQ ID NO: 10) elastase cleavable or substantially specific sequence is RLQLA(acetyl)L (SEQ ID NO: 11). In some embodiments, the plasmin cleavable or substantially specific sequence is RLQLKL (SEQ ID NO: 12). Thrombin selective substrates DPRSFL (SEQ ID NO: 13), PPRSFL (SEQ ID NO: 14), Norleucine-TPRSFL (SEQ ID NO: 15). In some embodiments, the chymase cleavable or substantially specific sequence GVAY|SGA (SEQ ID NO: 16). Urokinase-type plasminogen activator (uPA) and tissue plasminogen activator (tPA) cleavable or substantially specific sequence is YGRAAA (SEQ ID NO: 17). In some embodiments, the uPA cleavable or substantially specific sequence is YGPRNR (SEQ ID NO: 18).

In some embodiments, a linker consisting of one or more amino acids is used to join peptide sequence A (i.e., the sequence designed to prevent uptake into cells) and peptide sequence B (i.e., the TS). Generally the peptide linker will have no specific biological activity other than to join the molecules or to preserve some minimum distance or other spatial relationship between them. However, the constituent amino acids of the linker may be selected to influence some property of the molecule such as the folding, net charge, or hydrophobicity.

In some embodiments, the linker is flexible. In some embodiments, the linker is rigid.

In some embodiments, the linker comprises a linear structure. In some embodiments, the linker comprises a non-linear structure. In some embodiments, the linker comprises a branched structure. In some embodiments, the linker comprises a cyclic structure.

In some embodiments, X is about 5 to about 30 atoms in length. In some embodiments, X is about 6 atoms in length. In some embodiments, X is about 8 atoms in length. In some embodiments, X is about 10 atoms in length. In some embodiments, X is about 12 atoms in length. In some embodiments, X is about 14 atoms in length. In some embodiments, X is about 16 atoms in length. In some embodiments, X is about 18 atoms in length. In some embodiments, X is about 20 atoms in length. In some embodiments, X is about 25 atoms in length. In some embodiments, X is about 30 atoms in length.

In some embodiments, X is cleaved by thrombin. In some embodiments, the linker is substantially specific for thrombin.

In some embodiments, the linker has a formula selected from: DPRSFL (SEQ ID NO: 13), or PPRSFL (SEQ ID NO: 14).

In some embodiments, the linker binds peptide portion A (i.e., the peptide sequence which prevents cellular uptake) to peptide portion B (i.e., the MTS sequence) by a covalent linkage. In some embodiments, the covalent linkage comprises an ether bond, thioether bond, amine bond, amide bond, carbon-carbon bond, carbon-nitrogen bond, carbon-oxygen bond, or carbon-sulfur bond.

In some embodiments, X comprises a peptide linkage. The peptide linkage comprises L-amino acids and/or D-amino acids. In embodiments of the invention, D-amino acids are employed in order to minimize immunogenicity and nonspecific cleavage by background peptidases or proteases. Cellular uptake of oligo-D-arginine sequences is known to be as good as or better than that of oligo-L-arginines.

It will be understood that a linker disclosed herein may include non-standard amino acids, such as, for example, hydroxylysine, desmosine, isodesmosine, or other non-standard amino acids. A linker disclosed herein may include modified amino acids, including post-translationally modified amino acids such as, for example, methylated amino acids (e.g., methyl histidine, methylated forms of lysine, etc.), acetylated amino acids, amidated amino acids, formylated amino acids, hydroxylated amino acids, phosphorylated amino acids, or other modified amino acids. A linker disclosed herein may also include peptide mimetic moieties, including portions linked by non-peptide bonds and amino acids linked by or to non-amino acid portions.

In some embodiments, a MTS molecule disclosed herein comprises a single of linker Use of a single mechanism to mediate uptake of both imaging and therapeutic cargoes is particularly valuable, because imaging with noninjurous tracer quantities can be used to test whether a subsequent therapeutic dose is likely to concentrate correctly in the target tissue.

In some embodiments, a MTS molecule disclosed herein comprises a plurality of linkers. Where a MTS molecule disclosed herein includes multiple linkages X, separation of portion A from the other portions of the molecule requires cleavage of all linkages X Cleavage of multiple linkers X may be simultaneous or sequential Multiple linkages X may include linkages X having different specificities, so that separation of portion A from the other portions of the molecule requires that more than one condition or environment (“extracellular signals”) be encountered by the molecule Cleavage of multiple linkers X thus serves as a detector of combinations of such extracellular signals For example, a MTS molecule may include two linker portions Xa and Xb connecting basic portion B with acidic portion A Both linkers Xa and Xb must be cleaved before acidic portion A is separated from basic portion B allowing entry of portion B and cargo moiety C (if any) to enter a cell It will be understood that a linker region may link to either a basic portion B or a cargo moiety C independently of another linker that may be present, and that, where desired, more than two linker regions X may be included

Combinations of two or more linkers X may be used to further modulate the detection of multiple proteases with a single MTS molecule, as well as targeting and delivery of molecules to desired cells, tissue or regions. Combinations of extracellular signals are used to widen or narrow the specificity of the cleavage of linkers X if desired. Where multiple linkers X are linked in parallel, the specificity of cleavage is narrowed, since each linker X must be cleaved before portion. A may separate from the remainder of the molecule. Where multiple linkers X are linked in series, the specificity of cleavage is broadened, since cleavage on any one linker X allows separation of portion A from the remainder of the molecule For example, in order to detect either a protease OR hypoxia (i. e., to cleave X in the presence of either protease or hypoxia), a linker X is designed to place the protease-sensitive and reduction-sensitive sites in tandem, so that cleavage of either would suffice to allow separation of the acidic portion A Alternatively, in order to detect the presence of both a protease AND hypoxia (i e, to cleave X in the presence of both protease and hypoxia but not in the presence of only one alone), a linker X is designed to place the protease sensitive site between at least one pair of cysteines that are disulfide-bonded to each other In that case, both protease cleavage and disulfide reduction are required in order to allow separation of portion A.

One important class of signals is the hydrolytic activity of matrix metalloproteinases (MMPs), which are very important in the invasive migration of metastatic tumor cells. MMPs are also believed to play major roles in inflammation and stroke. MMPs are reviewed in Visse et al., Circ. Res. 92:827-839 (2003). MMPs may be used to cleave a linker X and so to allow separation of acidic portion A from portions B and C, allowing cellular uptake of cargo C so that cellular uptake of C is triggered by action of MMPs. Such uptake is typically in the vicinity of the MMPs that trigger cleavage of X. Thus, uptake of molecules having features of the invention are able to direct cellular uptake of cargo C to specific cells, tissues, or regions having active MMPs in the extracellular environment.

For example, a linker X that includes the amino-acid sequence PLGLAG (SEQ ID NO: 1) may be cleaved by the metalloproteinase enzyme MMP-2 (a major MMP in cancer and inflammation). Cleavage of such a linker X occurs between the central G and L residues, causing cell uptake to increase by 10 to 20-fold. A great deal is known about the substrate preferences of different MMPs, so that linkers X may be designed that are able to bias X to be preferentially sensitive to particular subclasses of MMPs, or to individual members of the large MMP family of proteinases. For example, in some embodiments, linkers X designed to be cleaved by membrane-anchored MMPs are particularly employed because their activity remains localized to the outer surface of the expressing cell. In alternative embodiments, linkers X designed to be cleaved by a soluble secreted MMP are employed where diffusion of cargo C away from the exact location of cleavage may be desired, thereby increasing the spatial distribution of the cargo. Other linkers X cleavable by other MMPs are discussed throughout the application.

Hypoxia is an important pathological signal. For example, hypoxia is thought to cause cancer cells to become more resistant to radiation and chemotherapy, and also to initiate angiogenesis. A linker X suitable for cleavage in or near tissues suffering from hypoxia enables targeting of portion B and C to cancer cells and cancerous tissues, infarct regions, and other hypoxic regions. For example, a linker X that includes a disulfide bond is preferentially cleaved in hypoxic regions and so targets cargo delivery to cells in such a region. In a hypoxic environment in the presence of, for example, leaky or necrotic cells, free thiols and other reducing agents become available extracellularly, while the 0₂ that normally keeps the extracellular environment oxidizing is by definition depleted. This shift in the redox balance should promote reduction and cleavage of a disulfide bond within a linker X. In addition to disulfide linkages which take advantage of thiol-disulfide equilibria, linkages including quinones that fall apart when reduced to hydroquinones may be used in a linker X designed to be cleaved in a hypoxic environment.

Necrosis often leads to release of enzymes or other cell contents that may be used to trigger cleavage of a linker X. A linker X designed for cleavage in regions of necrosis in the absence of hypoxia, for example, may be one that is cleaved by calpains or other proteases that may be released from necrotic cells. Such cleavage of linkers X by calpains would release the connected portions B-C from portion A, allowing cargo to be taken up by diseased cells and by neighboring cells that had not yet become fully leaky.

Acidosis is also commonly observed in sites of damaged or hypoxic tissue, due to the Warburg shift from oxidative phosphorylation to anaerobic glycolysis and lactic acid production. Such local acidity could be sensed either by making an acid-labile linker X (e.g., by including in X an acetal or vinyl ether linkage). Alternatively, or in addition, acidosis may be used as a trigger of cargo uptake by replacing some of the arginines within B by histidines, which only become cationic below pH 7.

Molecules having features of the invention are suitable for carrying different cargoes, including different types of cargoes and different species of the same types of cargo, for uptake into cells. For example, different types of cargo may include marker cargoes (e.g., fluorescent or radioactive label moieties) and therapeutic cargoes (e.g., chemotherapeutic molecules such as methotrexate or doxorubicin), or other cargoes. Where destruction of aberrant or diseased cells is therapeutically required, a therapeutic cargo may include a “cytotoxic agent,” i.e. a substance that inhibits or prevents the function of cells and/or causes destruction of cells. In some embodiments, a single molecule having features of the invention may include more than one cargo portion C so that a basic portion B may be linked to multiple cargoes C. Such multiple cargoes C may include marker cargoes, therapeutic cargoes, or other cargoes. Multiple cargo moieties may allow, for example, delivery of both a radioactive marker and an ultrasound or contrast agent, allowing imaging by different modalities. Alternatively, for example, delivery of radioactive cargo along with an anti-cancer agent, providing enhanced anticancer activity, or delivery of a radioactive cargo with a fluorescent cargo, allowing multiple means of localizing and identifying cells which have taken up cargo.

Delivery of cargo such as a fluorescent molecule may be used to visualize cells having a certain condition or cells in a region exhibiting a particular condition. For example, thrombosis (clot formation) may be visualized by designing a linker X to be cleaved by any of the many proteases in the blood clot formation cascade for delivery of a cargo including a fluorescent or other marker to the region. Similarly, complement activation may be visualized by designing a linker X to be cleaved by any one or more of the proteases in the complement activation cascades for delivery of a fluorescent or other marker to the region. Thus, fluorescent molecules are one example of a marker that may be delivered to target cells and regions upon release of a portion A upon cleavage of a linker X.

A molecule having features of the invention may include one or more linkers X so that an acidic portion A may be linked to portions B and C by one or more linkages. Such linkages connecting to portion A may be to portion B, to portion C, or to both portions B and C. Where a molecule having features of the invention includes multiple linkages X, separation of portion A from the other portions of the molecule requires cleavage of all linkages X. Cleavage of multiple linkers X may be simultaneous or sequential. Multiple linkages X may include linkages X having different specificities, so that separation of portion A from the other portions of the molecule requires that more than one condition or environment (“extracellular signals”) be encountered by the molecule. Cleavage of multiple linkers X thus serves as a detector of combinations of such extracellular signals. In some embodiments, MTS molecule having includes two linker portions Xa and Xb connecting basic portion B with acidic portion A. In some embodiments, a cyclic MTS molecule includes two linker regions Xa and Xb connecting basic portion B with acidic portion A. In some embodiments, both linkers Xa and Xb must be cleaved before acidic portion A is separated from basic portion B allowing entry of portion B and cargo portion C (if any) to enter a cell. It will be understood that a linker region may link to either a basic portion B or a cargo portion C independently of another linker that may be present, and that, where desired, more than two linker regions X may be included.

Combinations of two or more linkers X may be used to further modulate the targeting and delivery of molecules to desired cells, tissue or regions. Boolean combinations of extracellular signals can be detected to widen or narrow the specificity of the cleavage of linkers X if desired. Where multiple linkers X are linked in parallel, the specificity of cleavage is narrowed, since each linker X must be cleaved before portion A may separate from the remainder of the molecule. Where multiple linkers X are linked in series, the specificity of cleavage is broadened, since cleavage on any one linker X allows separation of portion A from the remainder of the molecule. For example, in order to detect either a protease OR hypoxia (i.e., to cleave X in the presence of either protease or hypoxia), a linker X is designed to place the protease-sensitive and reduction-sensitive sites in tandem, so that cleavage of either would suffice to allow separation of the acidic portion A. Alternatively, in order to detect the presence of both a protease AND hypoxia (i.e., to cleave X in the presence of both protease and hypoxia but not in the presence of only one alone), a linker X is designed to place the protease sensitive site between at least one pair of cysteines that are disulfide-bonded to each other. In that case, both protease cleavage AND disulfide reduction are required in order to allow separation of portion A.

D amino acids may be used in MTS molecules having features of the invention. For example, some or all of the peptides of portions A and B may be D-amino acids in some embodiments of the invention. In an embodiment of the invention suitable for delivering a detectable marker to a target cell, a MTS having features of the invention includes a contrast agent as cargo C attached to a basic portion B comprising 8 to 10 D-arginines. Acidic portion A may include D-amino acids as well. Similarly, a drug may be delivered to a cell by such molecules having a basic portion B including 8 to 10 D-arginines and an acidic portion A including acidic D-amino acids.

It will be understood that a MTS molecule having features of the invention may include non-standard amino acids, such as, for example, hydroxylysine, desmosine, isodesmosine, or other non-standard amino acids. A MTS molecule having features of the invention may include modified amino acids, including post-translationally modified amino acids such as, for example, methylated amino acids (e.g., methyl histidine, methylated forms of lysine, etc.), acetylated amino acids, amidated amino acids, formylated amino acids, hydroxylated amino acids, phosphorylated amino acids, or other modified amino acids. A MTS molecule having features of the invention may also include peptide mimetic moieties, including portions linked by non-peptide bonds and amino acids linked by or to non-amino acid portions. For example, a MTS molecule having features of the invention may include peptoids, carbamates, vinyl polymers, or other molecules having non-peptide linkages but having an acidic portion cleavably linked to a basic portion having a cargo moiety.

The linker portion X may be designed so that it is cleaved, for example, by proteolytic enzymes or reducing environment, as may be found near cancerous cells. Such an environment, or such enzymes, are typically not found near normal cells. In some embodiments, a cleavable linker X is designed to be cleaved near cancerous cells. In some embodiments, the cleavable linker is not cleaved near normal tissue. A capable of vetoing cellular uptake of a portion B, and of a portion B-C, blocking the entry of cargo into normal tissue.

In some embodiments, the linker portion X may be cleaved, for example, by proteolytic enzymes or reducing environment found near cancerous cells to deliver a marker or a drug to cancerous cells. In some embodiments, a MTS molecule with a cleavable linker X that is cleaved by proteolytic enzymes or by the reducing environment near cancer cells is able to facilitate cargo entry into diseased tissue. Thus, the selective cleavage of the linker X and the resulting separation of cargo C and basic portion B from acidic portion A allows the targeted uptake of cargo into cells having selected features (e.g., enzymes), or located near to, a particular environment. Thus, molecules having features of the invention are able to selectively deliver cargo to target cells without doing so to normal or otherwise non-targeted cells.

In some embodiments, a MTS disclosed herein has the formula (A-X-B)n-D, wherein C is a cargo moiety; A is a peptide with a sequence comprising 5 to 9 consecutive acidic amino acids, wherein the amino acids are selected from: aspartates and glutamates; B is a peptide with a sequence comprising 5 to 20 consecutive basic amino acids; X is a linker that is cleavable by thrombin; D is a dendrimer; and n is an integer between 1 and 20. In some embodiments, D comprises a cargo moiety.

In embodiments, a MTS molecule disclosed herein is a linear molecule. In embodiments, a MTS molecule disclosed herein is a cyclic molecule, as schematically illustrated in FIG. 1B of WO 2011/008996; incorporated herein by reference in its entirety. In embodiments, a MTS molecule disclosed herein comprises a cyclic portion and a linear portion.

A MTS disclosed herein may be of any length. In some embodiments, a MTS molecule disclosed herein is about 7 to about 40 amino acids in length, not including the length of a linker X and a cargo moiety C. In other embodiments, particularly where multiple non-acidic (in portion A) or non-basic (in portion B) amino acids are included in one or both of portions A and B, portions A and B of a MTS molecule disclosed herein may together be about 50, or about 60, or about 70 amino acids in length. A cyclic portion of a MTS molecule disclosed herein may include about 12 to about 60 amino acids, not including the length of a linker X and a cargo moiety C. For example, a linear MTS molecule disclosed herein may have a basic portion B having between about 5 to about 20 basic amino acids (between about 9 to about 16 basic amino acids) and an acidic portion A having between about 2 to about 20 acidic amino acids (e.g., between about 5 to about 20, between about 5 to about 9 acidic amino acids). In some particular embodiments, a MTS molecule disclosed herein may have a basic portion B having between about 9 to about 16 basic amino acids and between about 5 to about 9 acidic amino acids. In some embodiments, A is consecutive glutamates (i.e., EEEEEEEE, E9, eeeeeeee, or e9), B is nine consecutive arginines (i.e., RRRRRRRRR, R9, rrrrrrrrr, or r9), and X is PLGLAG (SEQ ID NO: 1).

In some embodiments, the MTS is selected from: Suc-e9-XDPRSFL-r9-c(Cy5)-CONH2; Suc-e9-ODPRSFL-r9-c(Cy5)-CONH2; and Suc-e9-Xdprsfl-r9-c(Cy5)-CONH2.

A MTS molecule disclosed herein may be of any length. In some embodiments, a MTS molecule disclosed herein is about 7 to about 40 amino acids in length, not including the length of a linker X and a cargo moiety C. In other embodiments, particularly where multiple non-acidic (in portion A) or non-basic (in portion B) amino acids are included in one or both of portions A and B, portions A and B of a MTS molecule disclosed herein may together be about 50, or about 60, or about 70 amino acids in length. A cyclic portion of a MTS molecule disclosed herein may include about 12 to about 60 amino acids, not including the length of a linker X and a cargo moiety.

For example, a linear MTS molecule disclosed herein may have a basic portion B having between about 5 to about 20 basic amino acids (in some embodiments between about 9 to about 16 basic amino acids) and an acidic portion A having between about 2 to about 20 acidic amino acids (e.g., between about 5 to about 20, between about 5 to about 9 acidic amino acids). In some embodiments, a MTS molecule disclosed herein may have a basic portion B having between about 9 to about 16 basic amino acids and between about 5 to about 9 acidic amino acids. In some embodiments, A is 9 consecutive glutamates (i.e., EEEEEEEE, E9, eeeeeeee, or e9), B is nine consecutive arginines (i.e., RRRRRRRRR, R9, rrrrrrrrr, or r9), and X is PLGLAG (SEQ ID NO: 1).

In some embodiments, the MTS molecule has a formula given below. It should be noted that in some instances the peptide sequence is given by the amino acid symbol and a number indicating the number of amino acids (for example, R9 translates to RRRRRRRRR or nine consecutive L-argmines; and r9 translates to nine consecutive D-argmines or rrrrrrrrr)

(SEQ ID NO: 39)
EDDDDKA-aca-R9-aca-C(F1)-CONH2
(SEQ ID NO: 40)
Fl-aca-CRRRRRRRRR-aca-EEEEEEEEEC-CONH2
(SEQ ID NO: 41)
Fl-aca-CEEEE-aca-RRRRRRRRRC-CONH2
(SEQ ID NO: 42)
H2N-EEEEEDDDDKA-aca-RRRRRRRRR-aca-C(Fl)-CONH2
(SEQ ID NO: 43)
H2N-EDDDDKA-aca-RRRRRRRRR-aca-C(Fl)-CONH2
(SEQ ID NO: 44)
H2N-EEEEEDDDDK ARRRRRRRRR-aca-C(Fl)-CONH2
(SEQ ID NO: 45)
H2N-EEDDDDKA-aca-rarrarr-aca-C(Fl)-CONH2
H2N-DDDDDDKARRRRRRRRR-aca-C(Fl)-CONH2
(SEQ ID NO: 46)
H2N-EEDDDDKAR-aca-RR-aca-RR-aca-RR-aca-RR-aca-
C(Fl)-CONH2
(SEQ ID NO: 47)
H2N-eeeeee-aca-PLGLAG-rrrrrrrrr-aca-c(Fl)-CONH2
EDA-aca-R,-aca-C(Fl)-CONH2
(SEQ ID NO: 48)
EDDDDKA-aca-R6-aca-C(DOX)-CONH2
(SEQ ID NO: 49)
EEEDDDEEEDA-aca-R9-aca-Y(12SI)-CONH2
ededdAAeeeDDDDKA-aca-R,,-aca-C(Fl)-CONH2
eddedededDDDDKA-aca-Rs-AGA-R6-aca-C(DOX)-CONH2
Ggedgddeeeeeeddeed-aca-PLGLAG-aca-R8-AAA-Ri2-
aca-C(Fl)-CONH2
eeddeeddKA-aca-R7-aca-C(Fl)-CONH2
eDDDDKA-aca-RGRGRRR-aca-C(Fl)-CONH2
eddddeeeeeee-aca-PLGLAGKA-aca-R10-aca-C(Fl)-CONH2
eeeeeeeeeeeeeeee-aca-DDDDKA-aca-R20-aca-C(Fl)-
CONH2
eeeeeeeeeddddd-aca-DDDDKA-aca-R, 7-aca-Y
(′2<iI)-CONH2
dddddddddddddddd-aca-PLGLAG-aca-R, 4-aca-C(DOX)-
CONH2
NH2-eeeeee-ahx-PLG
LAG-rrrrrrrrr-ahx-c(Fl)-CONH2, where ″ahx″
indicates ammohexanoic acid
(SEQ ID NO: 50)
EEEEEDDDDKAXRRRRRRRRRXC(FI)
(SEQ ID NO: 51)
EEEEEDDDDKARRRRRRRRRXC(Fl)
(SEQ ID NO: 52)
EDDDDKAXRRRRRRRRRXC(Fl)
(SEQ ID NO: 53)
EEDDDDKARXRRXRRXRRXRRXC(Fl)
(SEQ ID NO: 54)
DDDDDDKARRRRRRRRRXC(Fl)
EEDDDDKAXrrrrrrrrrXC(Fl)
eeeeeeXPLGLAGrrrrrrrrrXc(Fl)
UeeeeeeeeXPLGLAGrrrrrrrrrXk(Fl)
eeeeeeXPLGLAGrrrrrrrrrXc(Cy5)
UeeeeeeXPLGLAGrrrrrrrrrXc(Cy5)
UeeeeeeeeXPLGLACorturraXk(Cy5)
11-kDaPEG]XeeeeeeeeeXPLGLAGrarrarrXk(Cy5)
11-kDaPEG]XeeeeeeeeeXLALGPGrarrarrXk(Cy5)
Fl-XrarrarrXPLGLAGeeeeeeee-βAla
Fl-XrarrarrXSGRSAeeeeeeee-βAla
eeeeeeXSGRSAXrrrrrrrrrXc(Cy5)
Fl-rrrrrrrrrc-SS-ceeeeee
succinyl-e8-XPLGLAG-r9-Xk, where X denotes 6-
aminohexanoyI
[11 kDa PEG]-X-e9-XPLGLAG-r9
[11-kDa PEG]-X-e9-XPLGLAG-r9-Xk(Cy5)
H2N-e6-XPLGLAG-r9-Xc(Cy5)-CONH2, where
X ≡ aminohexanoic acid
H2N-eeeeee-(ahx)-PLG LAG-rrrrrrrrr-(ahx)-c
(Fluor)-CONH2
XeeeeeeeeeXPLGLAGrrrrrrrrXk
eeeeeeeeeXLALGPG-rrrrrrrrrXk(Cy5)
mPEG(11 kd)-S-CH2-CONH-ahx-e9-ahx-PLGLAG-r9-ahx-
k-CONH2 mPEG-S-CH2CONH-e9-ahx-PLGLAG-r9-
K[DOTA(Gd)]-CONH2
(11 KDa-mPEG)-e9-XPLGLAG-r9-[DPK-99mTc(CO)3]
(70 KDa-dextran)-e9-XPLGLAG-r9-[DPK-99mTc(CO)3]
murine serum albumin)-e9-XPLGLAG-r9-[DPK-
99mTc(CO)3]
(PAMAM generation 5 dendrimer)-e9-XPLGLAX-r9-
[DPK-″mTc(CO)3]
(70 KDa dextran)-e9-XPLGLAX-r9-(DOTA-′11In)
(11-KDa-mPEG)-e9-XPLGLAG-r9-K(DOTA-Gd)
Suc9-(70 KDa dextran)-e9-XPLGLAG-r9-K(DOT A-Gd)
Suc9-(70 KDa dextran)-e9-XPLGLAX-r9-K(DOT A-Gd)
Suc9-(70 KDa dextran)-e9-XPLGLAG-r9-K(DOT A-Gd)
cyclic[succinoyl-PLGLAG-c(11 KDa-mPEG)-e9-
XPLGLAG-r9-K]-k(Cy5)
Cy5-X-e6-XPLGLAG-r9-Xk(Cy5)
Cy7-X-e6-XPLGLAG-r9-Xk(Cy5)
11 KDa mPEG-e9-PLGLAG-r9
Ac-r9-k-NH2
mPEG(11 kd)-e9-XPLGLAG-r9-Xk-NH2
e9-XPLGLAG-r9-Xk-NH2
FAM-e9-dPEG(6)-SGRFPKTVHTA-r9-(D-cys)-NH2
FAM-e9-dPEG(6)-SGSNPFKYHTA-r9-(D-cys)-NH2
FAM-e9-dPEG(6)-SGGPQGIAGTA-r9-(D-cys)-NH2
FAM-e9-dPEG(6)-SGPLKITRTA-r9-(D-cys)-NH2
FAM-e9-dPEG(6)-SGIPFFMTTA-r9-(D-cys)-NH2
FAM-e9-dPEG(6)-SGMGPWFMHTA-r9-(D-cys)-NH2
FAM-e9-dPEG(6)-SGSNPYK-YTA-r9-(D-cys)-NH2
FAM-e9-dPEG(6)-SGSNPKG-YTA-r9-(D-cys)-NH2
FAM-e9-dPEG(6)-SGSNPYG-YTA-r9-(D-cys)-NH2
FAM-e9-dPEG(6)-SG- XXXXXX-TA-r9-(D-cys)-NH2

TABLE 1

| Cap | Macromolecule | Polyanion | P4 | P3 | P2 | P1 | P1′ | P2′ | P3′ . . . Pn′ | Polycation | Cargo | C-term |
|---|---|---|---|---|---|---|---|---|---|---|---|---|
| Suc9 | Dextran | e9 | X | P | L | G | L | A | G | r9 | K[DOTA(Gd)] | NH₂ |
| | (70 KDa) | | | | | | | | | | | |
| Suc | — | e9 | X | P | L | G | C(Me) | A | X | r9 | DPK | NH₂ |
| Suc | — | e9 | X | P | ThienylAla | G | C(Me) | A | X | r9 | DPK | NH₂ |
| Suc | — | e9 | X | P | F(4---Cl) | G | C(Me) | A | X | r9 | DPK | NH₂ |
| Suc | — | e8 | X | P | L | G | L | A | G | r9 | c[Cy5] | NH₂ |
| Suc | — | e8 | X | P | F(4---Cl) | G | C(Me) | M | X | r9 | c[Cy5] | NH₂ |
| Suc | — | e8 | X | P | F(4---Cl) | G | C(Me) | Y | X | r9 | c[Cy5] | NH₂ |
| Suc | — | e8 | X | P | F(4---Cl) | G | C(Me) | R | X | r9 | c[Cy5] | NH₂ |
| Suc | — | e8 | X | P | F(4---Cl) | G | C(Me) | PhGly | X | r9 | c[Cy5] | NH₂ |
| Suc | — | e8 | X | P | F(4---Cl) | G | C(Me) | C(Me) | X | r9 | c[Cy5] | NH₂ |
| — | Albumin | e9 | X | P | L | G | L | A | X | r9 | DPK | NH₂ |
| Suc | — | e8 | X | P | C(Me) | G | C(Me) | A | X | r9 | c[Cy5] | NH₂ |
| Suc | — | e8 | X | P | ThienylAla | G | C(Me) | A | X | r9 | c[Cy5] | NH₂ |
| Suc | — | e8 | X | P | F(4---Cl) | G | C(Me) | A | X | r9 | c[Cy5] | NH₂ |
| Suc | — | e8 | X | P | K(Dnp) | G | C(Me) | A | X | r9 | c[Cy5] | NH₂ |
| — | Albumin | e9 | X | P | L | G | L | A | X | r9 | DPK | NH₂ |
| Suc | — | e8 | X | P | L | G | C(Me) | M | X | r9 | c[Cy5] | NH₂ |
| Suc | — | e8 | X | P | L | G | C(Me) | Y | X | r9 | c[Cy5] | NH₂ |
| Suc127 | PAMAM-Gen5 | e9 | X | P | L | G | L | A | X | r9 | DPK | NH₂ |
| Suc | — | e8 | X | P | L | G | C(Me) | A | X | r9 | c[Cy5] | NH₂ |
| Suc9 | Dextran | e9 | X | P | L | G | L | A | G | r9 | K[DOTA(Gd)] | NH₂ |
| | (70 KDa) | | | | | | | | | | | |
| Suc127 | PAMAM-Gen5 | e9 | X | P | L | G | L | A | X | r9 | k[Cy5] | NH₂ |
| — | — | — | — | — | — | — | — | — | — | r9 | Xc[Cy5] | NH₂ |
| Ac127 | PAMAM-Gen5 | e9 | X | P | L | G | L | A | X | r9 | k[Cy5] | NH₂ |
| Suc | — | e8 | X | P | L | G | L | F(4--- | A | Xr9 | k[Cy5] | NH₂ |
| | | | | | | | | NO2) | | | | |
| Suc127 | PAMAM-Gen5 | e9 | X | P | L | G | L | A | X | r9 | k[Cy5] | NH₂ |
| Suc63 | PAMAM-Gen4 | e9 | X | P | L | G | L | A | X | r9 | k[Cy5] | NH₂ |
| — | Albumin | e9 | X | P | L | G | L | A | G | r9 | DPK | NH₂ |
| Suc136 | Dextran | e9 | X | P | L | G | L | A | X | r9 | k[Cy5] | NH₂ |
| | (86 KDa) | | | | | | | | | | | |
| Suc | — | e8 | X | P | L | G | L | A | X | r9 | k[Cy5] | NH₂ |
| Suc9 | Dextran | e9 | X | P | L | G | L | A | X | r9 | K[DOTA(Gd)] | NH₂ |
| | (70 KDa) | | | | | | | | | | | |
| Suc9 | Dextran | e9 | X | P | L | G | L | A | X | r9 | K[DOTA(Gd)] | NH₂ |
| | (70 KDa) | | | | | | | | | | | |
| Suc9 | Dextran | e9 | X | P | L | G | L | A | G | r9 | k[Cy5] | NH₂ |
| | (70 KDa) | | | | | | | | | | | |
| Suc9 | Dextran | e9 | X | P | L | G | L | A | G | r9 | DPK | NH₂ |
| | (70 KDa) | | | | | | | | | | | |
| Suc | — | e8 | X | p | l | g | l | a | g | r9 | k[Cy5] | NH₂ |
| — | Albumin | e9 | X | p | l | g | l | a | g | r9 | k[Cy5] | NH₂ |
| Suc9 | Dextran | e9 | X | p | l | g | l | a | g | r9 | k[Cy5] | NH₂ |
| Suc97 | Dextran | e9 | X | P | L | G | L | A | G | r9 | k[Cy5] | NH₂ |
| | (500 KDa) | | | | | | | | | | | |
| — | Albumin | e9 | X | P | L | G | L | A | G | r9 | k[Cy5] | NH₂ |
| Suc9 | Dextran | e9 | X | P | L | G | L | A | G | r9 | k[Cy5] | NH₂ |
| | (70 KDa) | | | | | | | | | | | |
| — | Albumin | e9 | X | P | L | G | L | A | G | r9 | k[Cy5] | NH₂ |
| Suc | | e8 | X | P | L | G | L | A | X | r9 | k[Cy5] | NH₂ |
| Suc9 | Dextran | e9 | X | P | L | G | L | A | G | r9 | k[Cy5] | NH₂ |
| | (70 KDa) | | | | | | | | | | | |
| — | Albumin | e9 | X | P | L | G | L | A | G | r9 | k[Cy5] | NH₂ |
| — | Albumin | e9 | X | P | L | G | L | A | G | r9 | k[Cy5] | NH₂ |
| — | Albumin | e9 | X | P | L | G | L | A | G | r9 | k[Cy5] | NH₂ |
| Suc9 | Dextran | e9 | X | P | L | G | L | A | G | r9 | k[Cy5] | NH₂ |
| | (70 KDa) | | | | | | | | | | | |
| Suc | nonconj. | e8 | X | P | L | G | L | A | G | r9X | k[Cy5] | NH₂ |
| | Albumin | | | | | | | | | | | |
| Suc | — | e8 | X | P | L | G | L | A | G | r9X | k[Cy5] | NH₂ |
| — | Albumin | e9 | X | P | L | G | L | A | G | r9 | k[Cy5] | NH₂ |
| — | mPEG | e9 | x | p | l | g | l | a | g | r9X | k[Cy5] | NH₂ |
| | (5 KDa) | | | | | | | | | | | |
| — | mPEG | e9 | X | P | L | G | L | A | G | r9 | K[DOTA(Gd)] | NH₂ |
| | (11 KDa) | | | | | | | | | | | |
| — | mPEG | e10 | X | P | L | G | F(4- | A | Q | Xr9 | k[Cy5] | NH₂ |
| | (11 KDa) | | | | | | NO₂) | | | | | |
| — | mPEG | e10 | X | P | L | G | C(Me) | W | A | Qr9 | k[Cy5] | NH₂ |
| | (11 KDa) | | | | | | | | | | | |
| — | mPEG | e9 | X | P | L | G | C(Me) | W | A | Qr9 | k[Cy5] | NH₂ |
| | (11 KDa) | | | | | | | | | | | |

In some embodiments, cargo C may be a fluorescent molecule such as fluorescein. Fluorescent cargo moieties enable easy measurement by fluorescence microscopy or flow cytometry in unfixed cultured cells. However, oligoarginine sequences, such as make up portion B, have been demonstrated to import a very wide varieties of cargoes C, ranging from small polar molecules to nanoparticles and vesicles (Tung & Weissleder, Advanced Drug Delivery Reviews 55: 281-294 (2003)). Thus, in embodiments of the invention, a cargo portion C may be any suitable cargo moiety capable of being taken up by a cell while connected to a basic portion B.

For example, for in vivo imaging purposes, C may be labeled with a positron-emitting isotope (e.g. ¹⁸F)) for positron emission tomography (PET), gamma-ray isotope (e.g. ^{99m}Tc) for single photon emission computed tomography (SPECT), a paramagnetic molecule or nanoparticle (e.g. Gd³⁺ chelate or coated magnetite nanoparticle) for magnetic resonance imaging (MRI), a near-infrared fluorophore for near-infra red (near-IR) imaging, a luciferase (firefly, bacterial, or coelenterate) or other luminescent molecule for bioluminescence imaging, or a perfluorocarbon-filled vesicle for ultrasound. For therapeutic purposes, for example, suitable classes of cargo include but are not limited to: a) chemotherapeutic agents such as doxorubicin, mitomycin, paclitaxel, nitrogen mustards, etoposide, camptothecin, 5-fluorouracil, etc.; b) radiation sensitizing agents such as porphyrins for photodynamic therapy, or ¹⁰I3 clusters or ¹⁵⁷Gd for neutron capture therapy; or c) peptides or proteins that modulate apoptosis, the cell cycle, or other crucial signaling cascades. Existing chemotherapeutic drugs may be used, although they may not be ideal, because they have already been selected for some ability to enter cells on their own. In some embodiments of the molecules of the invention, cargoes that are unable to enter or leave cells without the help of the polybasic portion B may be employed.

Cargo C may include a radioactive moiety, for example a radioactive isotope such as ²¹¹At, ¹³¹I, ¹²⁵I, ⁹⁰Y, ¹⁸⁶Re, ¹⁸⁸Re, ¹⁵³Sm, ²¹²Bi, ³²P, radioactive isotopes of Lu, and others.

Cargo portion C may include a fluorescent moiety, such as a fluorescent protein, peptide, or fluorescent dye molecule. Common classes of fluorescent dyes include, but are not limited to, xanthenes such as rhodamines, rhodols and fluoresceins, and their derivatives; bimanes; coumarins and their derivatives such as umbelliferone and aminomethyl coumarins; aromatic amines such as dansyl; squarate dyes; benzofurans; fluorescent cyanines; carbazoles; dicyanomethylene pyranes, polymethine, oxabenzanthrane, xanthene, pyrylium, carbostyl, perylene, acridone, quinacridone, rubrene, anthracene, coronene, phenanthrecene, pyrene, butadiene, stilbene, lanthanide metal chelate complexes, rare-earth metal chelate complexes, and derivatives of such dyes. Fluorescent dyes are discussed, for example, in U.S. Pat. No. 4,452,720, U.S. Pat. No. 5,227,487, and U.S. Pat. No. 5,543,295. Cargo can also include detection agents.

A cargo portion C may include a fluorescein dye. Typical fluorescein dyes include, but are not limited to, 5-carboxyfluorescein, fluorescein-5-isothiocyanate and 6-carboxyfluorescein; examples of other fluorescein dyes can be found, for example, in U.S. Pat. No. 6,008,379, U.S. Pat. No. 5,750,409, U.S. Pat. No. 5,066,580, and U.S. Pat. No. 4,439,356. A cargo portion C may include a rhodamine dye, such as, for example, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, rhodamine 101 sulfonyl chloride (sold under the tradename of TEXAS RED®), and other rhodamine dyes. Other rhodamine dyes can be found, for example, in U.S. Pat. No. 6,080,852, U.S. Pat. No. 6,025,505, U.S. Pat. No. 5,936,087, U.S. Pat. No. 5,750,409. A cargo portion C may include a cyanine dye, such as, for example, Cy3, Cy3B, Cy3.5, Cy5, Cy5.5, Cy7.

Some of the above compounds or their derivatives will produce phosphorescence in addition to fluorescence, or will only phosphoresce. Some phosphorescent compounds include porphyrins, phthalocyanines, polyaromatic compounds such as pyrenes, anthracenes and acenaphthenes, and so forth, and may be, or may be included in, a cargo portion C. A cargo portion C may also be or include a fluorescence quencher, such as, for example, a (4-dimethylamino-phenylazo)benzoic acid (DABCYL) group.

In some embodiments, a cargo moiety is all or part of a molecular beacon. In some embodiments a cargo moeity is combined with a quencher moiety Q to form all or part of a molecular beacon. As used herein, “molecular beacon” means a pair of connected compounds having complementary regions with a fluorophore and a fluorescent quencher so that the fluorescence of the fluorophore is quenched by the quencher. One or both of the complementary regions may be part of the cargo moiety. Where only one of the complementary regions (e.g., the fluorescent moiety) is part of the cargo moiety, and where the quencher moiety is part of the linker X or the acidic portion A, then cleavage of the linker X will allow fluorescence of the fluorescent portion and detection of the cleavage. Upon cellular uptake, the fluorescent portion of a molecular beacon will allow detection of the cell. For example, a quencher Q may be attached to an acidic portion A to form a MTS molecule having features of the invention Q-A-X-B-C where cargo is fluorescent and is quenched by Q The quenching of the cargo moiety by Q is relieved upon cleavage of X, allowing fluorescent marking of a cell taking up portion B-C The combination of fluorescence dequenching and selective uptake should increase contrast between tissues able to cleave X compared to those that cannot cleave X.

A pair of compounds may be connected to form a molecular beacon or FRET pair, having complementary regions with a fluorophore and a fluorescent quencher associated together so that the fluorescence of the fluorophore is quenched by the quencher. Such pairs can be useful as detection agents and any fluorescent pairs known or described herein can be employed with the present invention. One or both of the complementary regions may be part of the cargo portion C. Where only one of the complementary regions (e.g., the fluorescent moiety) is part of the cargo portion C, and where the quencher moiety is part of the linker X or the acidic portion A, then cleavage of the linker X will allow fluorescence of the fluorescent portion and detection of the cleavage. Upon cellular uptake, the fluorescent portion of a molecular beacon will allow detection of the cell. For example, a quencher Q may be attached to an acidic portion A to form a MTS molecule having features of the invention Q-A-X-B C where cargo C is fluorescent and is quenched by Q. The quenching of C by Q is relieved upon cleavage of X, allowing fluorescent marking of a cell taking up portion B-C. The combination of fluorescence dequenching and selective uptake should increase contrast between tissues able to cleave X compared to those that cannot cleave X.

In some embodiments, C and/or Q and C for all or part of a donor:acceptor FRET pair or a BRET (bioluminescence resonance energy transfer) pair. Donors can include any appropriate molecules listed herein or known in the art and as such include but are not limited to FITC; Cy3; EGFP; cyan fluorescent protein (CFP); EGFP; 6-FAM; fluorescein, IAEDANS, EDANS and BODIPY FL. Acceptors can include any appropriate molecules listed herein or known in the art and as such include but are not limited to TRITC; Cy5; Cy3; YFP; 6-FAM; LC Red 640; Alexa Fluor 546; fluorescein; tetramethylrhodamine; Dabcyl (acceptor); BODIPY FL; QSY 7 and QSY 9 dyes. Exemplary FRET pairs can include but are not limited to CFP:YFP; Cy5:Cy7; FITC:TRITC; Cy3:Cy5; EGFP:Cy3; EGFP:YFP; 6-FAM:LC Red 640 or Alexa Fluor 546; fluorescein:tetramethylrhodamine; IAEDANS:fluorescein; EDANS:Dabcyl; fluorescein:fluorescein; BODIPY FL:BODIPY FL; and fluorescein:QSY 7 and QSY 9 dyes.

In some embodiments, the cargo moiety C and/or quencher moeity Q are a fluorescent moiety including but not limited to a fluorescent protein, peptide, or fluorescent dye molecule. Common classes of fluorescent dyes include, but are not limited to, xanthenes such as rhodamines, rhodols and fluoresceins, and their derivatives; bimanes; coumarins and their derivatives such as umbelliferone and aminomethyl coumarins; aromatic amines such as dansyl; squarate dyes; benzofurans; fluorescent cyanines; carbazoles; dicyanomethylene pyranes, polymethine, oxabenzanthrane, xanthene, pyrylium, carbostyl, perylene, acridone, quinacridone, rubrene, anthracene, coronene, phenanthrecene, pyrene, butadiene, stilbene, lanthanide metal chelate complexes, rare-earth metal chelate complexes, and derivatives of such dyes. Fluorescent dyes are discussed, for example, in U.S. Pat. No. 4,452,720, U.S. Pat. No. 5,227,487, and U.S. Pat. No. 5,543,295.

In some embodiments, a cargo moiety C and/or quencher moeity Q are fluorescein dyes. Typical fluorescein dyes include, but are not limited to, 5-carboxyfluorescein, fluorescein-5-isothiocyanate and 6-carboxyfluorescein; examples of other fluorescein dyes can be found, for example, in U.S. Pat. No. 6,008,379, U.S. Pat. No. 5,750,409, U.S. Pat. No. 5,066,580, and U.S. Pat. No. 4,439,356. A cargo moiety C may include a rhodamine dye, such as, for example, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, rhodamine 101 sulfonyl chloride (sold under the tradename of TEXAS RED®), and other rhodamine dyes. Other rhodamine dyes can be found, for example, in U.S. Pat. No. 6,080,852, U.S. Pat. No. 6,025,505, U.S. Pat. No. 5,936,087, U.S. Pat. No. 5,750,409. A cargo moiety C may include a cyanine dye, such as, for example, Cy3, Cy3B, Cy3.5, Cy5, Cy5.5, Cy7.

In some embodiments, cargo moiety C and/or quencher moeity Q are fluorophores. Fluorophores are commercially available and any known and/or commercially available fluorophore can be employed as the cargo moiety C detectable entity for the present invention. In some embodiments, the fluorophore exhibits green fluorescence (such as for example 494 nm/519 nm), orange fluorescence (such as for example 554 nm/570 nm), red fluorescence (such as for example 590 nm/617 nm), or far red fluorescence (such as for example 651 nm/672 nm) excitation/emission spectra. In some embodiments, the fluorophore is a fluorophore with excitation and emission spectra in the range of about 350 nm to about 775 nm. In some embodiments the excitation and emission spectra are about 346 nm/446 nm, about 494 nm/519 nm, about 554 nm/570 nm, about 555 nm/572 nm, about 590 nm/617 nm, about 651 nm/672 nm, about 679 nm/702 nm or about 749 nm/775 nm. In some embodiments, the fluorophore can include but is not limited to AlexaFluor 3, AlexaFluor 5, AlexaFluor 350, AlexaFluor 405, AlexaFluor 430, AlexaFluor 488, AlexaFluor 500, AlexaFluor 514, AlexaFluor 532, AlexaFluor 546, AlexaFluor 555, AlexaFluor 568, AlexaFluor 594, AlexaFluor 610, AlexaFluor 633, AlexaFluor 647, AlexaFluor 660, AlexaFluor 680, AlexaFluor 700, and AlexaFluor 750 (Molecular Probes AlexaFluor dyes, available from Life Technologies, Inc. (USA)). In some embodiments, the fluorophore can include but is not limited to Cy dyes, including Cy2, Cy3, Cy3B, Cy3.5, Cy5, Cy5.5 and Cy7 (available from Lumiprobes). In some embodiments the fluorophore can include but is not limited to DyLight 350, DyLight 405, DyLight 488, DyLight 550, DyLight 594, DyLight 633, DyLight 650, DyLight 680, DyLight 750 and DyLight 800 (available from Thermo Scientific (USA)). In some embodiments, the fluorophore can include but is not limited to a FluoProbes 390, FluoProbes 488, FluoProbes 532, FluoProbes 547H, FluoProbes 594, FluoProbes 647H, FluoProbes 682, FluoProbes 752 and FluoProbes 782, AMCA, DEAC (7-Diethylaminocoumarin-3-carboxylic acid); 7-Hydroxy-4-methylcoumarin-3; 7-Hydroxycoumarin-3; MCA (7-Methoxycoumarin-4-acetic acid); 7-Methoxycoumarin-3; AMF (4′-(Aminomethyl)fluorescein); 5-DTAF (5-(4,6-Dichlorotriazinyl)aminofluorescein); 6-DTAF (6-(4,6-Dichlorotriazinyl)aminofluorescein); 6-FAM (6-Carboxyfluorescein), 5(6)-FAM cadaverine; 5-FAM cadaverine; 5(6)-FAM ethylenediamme; 5-FAM ethylenediamme; 5-FITC (FITC Isomer I; fluorescein-5-isothiocyanate); 5-FITC cadaverin; Fluorescein-5-maleimide; 5-IAF (5-Iodoacetamidofluorescein); 6-JOE (6-Carboxy-4′,5′-dichloro-2′,7′-dimethoxyfluorescein); 5-CR110 (5-Carboxyrhodamine 110); 6-CR110 (6-Carboxyrhodamine 110); 5-CR6G (5-Carboxyrhodamine 6G); 6-CR6G (6-Carboxyrhodamine 6G); 5(6)-Caroxyrhodamine 6G cadaverine; 5(6)-Caroxyrhodamine 6G ethylenediamme; 5-ROX (5-Carboxy-X-rhodamine); 6-ROX (6-Carboxy-X-rhodamine); 5-TAMRA (5-Carboxytetramethylrhodamine); 6-TAMRA (6-Carboxytetramethylrhodamine); 5-TAMRA cadaverine; 6-TAMRA cadaverine; 5-TAMRA ethylenediamme; 6-TAMRA ethylenediamme; 5-TMR C6 malemide; 6-TMR C6 malemide; TR C2 malemide; TR cadaverine; 5-TRITC; G isomer (Tetramethylrhodamine-5-isothiocyanate); 6-TRITC; R isomer (Tetramethylrhodamine-6-isothiocyanate); Dansyl cadaverine (5-Dimethylaminonaphthalene-1-(N-(5-aminopentyl))sulfonamide); EDANS C2 maleimide; fluorescamine; NBD; and pyrromethene and derivatives thereof.

Some of the above compounds or their derivatives will produce phosphorescence in addition to fluorescence, or will only phosphoresce. Some phosphorescent compounds include porphyrins, phthalocyanines, polyaromatic compounds such as pyrenes, anthracenes and acenaphthenes, and so forth, and may be, or may be included in, a cargo moiety. A cargo moiety may also be or include a fluorescence quencher, such as, for example, a (4-dimethylamino-phenylazo)benzoic acid (DABCYL) group.

In some embodiments, a cargo moiety is a fluorescentl label. In some embodiments, a cargo moiety C and/or quencher moeity Q is indocarbocyanine dye, Cy5, Cy5.5, Cy7, IR800CW, or a combination thereof. In some embodiments, a cargo moiety is a MRI contrast agent. In some embodiments, a cargo moiety is Gd complex of [4,7,10-tris(carboxymethyl)-1,4,7,10-tetraazacyclododec-1-yl]acetyl.

Cargo C may include a chemotherapeutic moiety, such as a chemical compound useful in the treatment of cancer, or other therapeutic moiety, such as an agent useful in the treatment of ischemic tissue, or of necrotic tissue, or other therapeutic agent.

Multiple membrane translocation signals (MTS) have been identified. For example, the Tat protein of the human immunodeficiency virus 1 (HIV-1) is able to enter cells from the extracellular environment. A domain from Antennapedia homeobox protein is also able to enter cells.

Molecules comprising a MTS may also be used to carry other molecules into cells along with them. The most important MTS are rich in amino acids such as arginine with positively charged side chains. Molecules transported into cell by such cationic peptides may be termed “cargo” and may be reversibly or irreversibly linked to the cationic peptides.

The uptake facilitated by molecules comprising a MTS is currently without specificity, enhancing uptake into most or all cells. It is desirable to have the ability to target the delivery of cargo to a type of cell, or to a tissue, or to a location or region within the body of an animal. Accordingly, a need for a MTS molecule with increased in vivo circulation has been identified.

In some embodiments, a MTS molecule disclosed herein has the formula A-X-B-C, wherein C is a cargo moiety; A is a peptide with a sequence comprising 5 to 9 consecutive acidic amino acids, wherein the amino acids are selected from: aspartates and glutamates; B is a peptide with a sequence comprising 5 to 20 consecutive basic amino acids; and X is a linker that is cleavable by thrombin.

In some embodiments, a MTS molecule disclosed herein has the formula A-X-B-C, wherein C is a cargo moiety; A is a peptide with a sequence comprising 5 to 9 consecutive acidic amino acids, wherein the amino acids are selected from: aspartates and glutamates; B is a peptide with a sequence comprising 5 to 20 consecutive basic amino acids; and X is a linker that is cleavable by thrombin.

In some embodiments, a MTS molecule disclosed herein has the formula (A-X-B-C)n-M, wherein C is a cargo moiety; A is a peptide with a sequence comprising 5 to 9 consecutive acidic amino acids, wherein the amino acids are selected from: aspartates and glutamates; B is a peptide with a sequence comprising 5 to 20 consecutive basic amino acids; X is a linker that is cleavable by thrombin; M is a macromolecular carrier; and n is an integer between 1 and 20.

In some embodiments, a MTS molecule disclosed herein has the formula (A-X-B)n-D, wherein C is a cargo moiety; A is a peptide with a sequence comprising 5 to 9 consecutive acidic amino acids, wherein the amino acids are selected from: aspartates and glutamates; B is a peptide with a sequence comprising 5 to 20 consecutive basic amino acids; X is a linker that is cleavable by thrombin; D is a dendrimer; and n is an integer between 1 and 20. In some embodiments, D comprises a cargo moiety. See Example 1 for methods of attaching a label to a MTS molecule.

MTS molecules disclosed herein are suitable for carrying different cargoes, including different types of cargoes and different species of the same types of cargo, for uptake into cells. For example, different types of cargo may include marker cargoes (e.g., fluorescent or radioactive label moieties) and therapeutic cargoes (e.g., chemotherapeutic molecules such as methotrexate or doxorubicin), or other cargoes. Where destruction of aberrant or diseased cells is therapeutically required, a therapeutic cargo may include a “cytotoxic agent,” i.e., a substance that inhibits or prevents the function of cells and/or causes destruction of cells.

Delivery of cargo such as a fluorescent molecule may be used to visualize cells having a certain condition or cells in a region exhibiting a particular condition For example, thrombosis (clot formation) may be visualized by designing a linker X to be cleaved by thrombin Thus, fluorescent molecules are one example of a marker that may be delivered to target cells and regions upon release of a portion A upon cleavage of a linker X

In some embodiments, the cargo moiety is selected from an imaging agent, a therapeutic agent, a lipid, a detection agent or a combination thereof.

In some embodiments, the cargo portion comprises at least two cargo moieties In some embodiments, C comprises a marker cargo and a therapeutic cargo Multiple cargo moieties may allow, for example, delivery of both a radioactive marker and an ultrasound or contrast agent, allowing imaging by different modalities Alternatively, for example, delivery of radioactive cargo along with an anti cancer agent, providing enhanced anticancer activity, or delivery of a radioactive cargo with a fluorescent cargo, allowing multiple means of localizing and identifying cells which have taken up cargo.

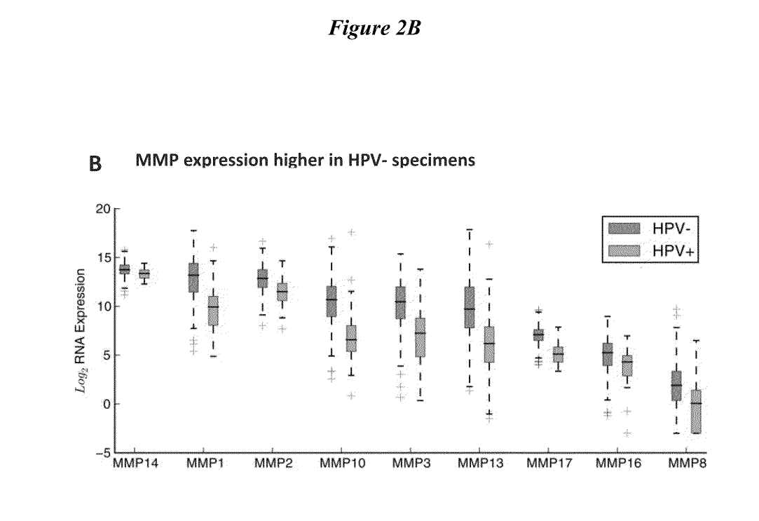

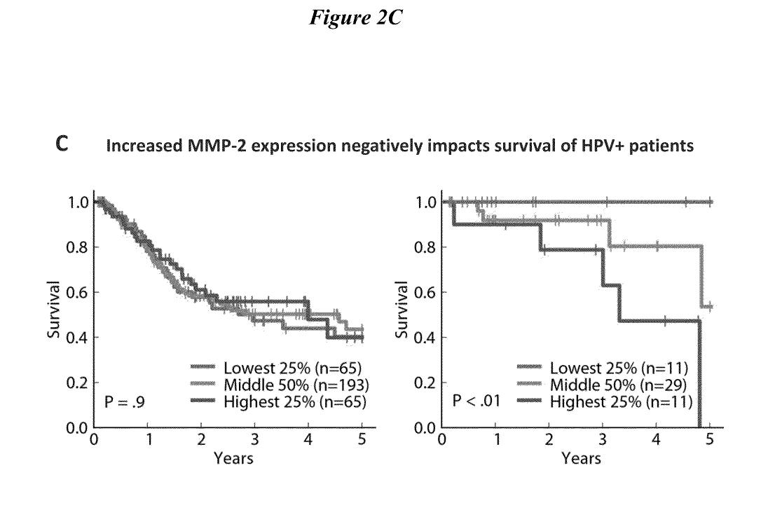

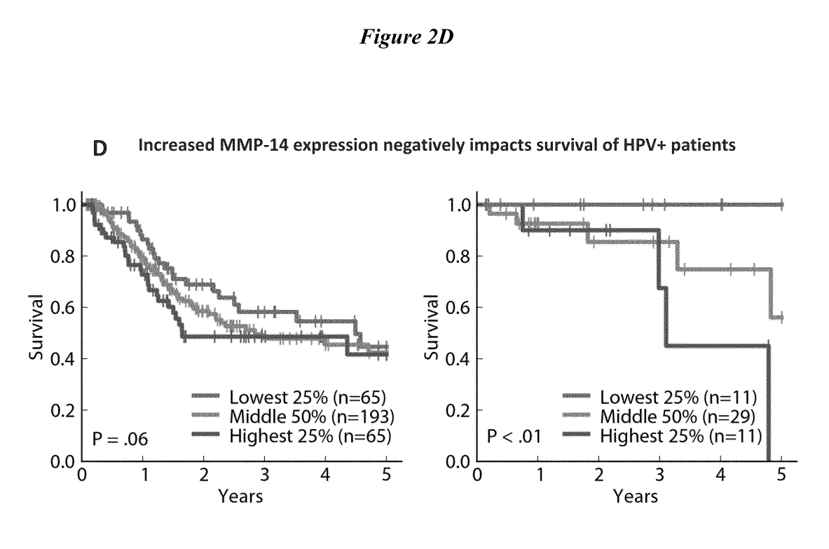

The cargo moiety is attached to B in any location or orientation The cargo moiety need not be located at an opposite end of portion B than a linker X Any location of attachment of the cargo moiety to B is acceptable as long as that attachment remains after X is cleaved For example, the cargo moiety may be attached to or near to an end of portion B with linker X attached to an opposite end of portion B as illustrated in FIGS. 2A and 2B The cargo moiety may also be attached to or near to an end of portion B with linker X attached to or near to the same end of portion B.

Wherein the molecule comprises a dendrimer, the cargo is attached directly to D By way of non-limiting example, the cargo is attached as follows (A-X-B)n-D-cargo.

In some embodiments, a cargo moiety is a fluorescent molecule such as fluorescein Fluorescent cargo moieties enable easy measurement by fluorescence microscopy or flow cytometry in unfixed cultured cells.

In some embodiments, a cargo moiety is labeled with a positron-emitting isotope (e g, 18F) for positron emission tomography (PET), gamma-ray isotope (e g, 99 mTc) for single photon emission computed tomography (SPECT), a paramagnetic molecule or nanoparticle (e g, Gd3+ chelate or coated magnetite nanoparticle) for magnetic resonance imaging (MRI), a near-infrared fluorophore for near-infra red (near-IR) imaging, a luciferase (firefly, bacterial, or coelenterate) or other luminescent molecule for bioluminescence imaging, or a perfluorocarbon-filled vesicle for ultrasound.

In some embodiments, a cargo moiety is a radioactive moiety, for example a radioactive isotope such as 21 1At, π iI, 12SI, 90Y, 186Re, 188Re, ‘“Sm, 212Bi, 12P, radioactive isotopes of Lu, and others.

In some embodiments, a cargo moiety is a therapeutic agent, such as a chemical compound useful in the treatment of cancer, ischemic tissue, or necrotic tissue.

For therapeutic purposes, for example, suitable classes of cargo include but are not limited to a) chemotherapeutic agents, b) radiation sensitizing agents, or c) peptides or proteins that modulate apoptosis, the cell cycle, or other crucial signaling cascades.

In some embodiments, a cargo moiety is an agent that treats a cardiovascular disorder In some embodiments, the cargo moiety is a niacin, a fibrate, a statin, an Apo-Al mimetic peptide, an apoA-I transciptional up-regulator, an ACAT inhibitor, a CETP modulator, or a combination thereof, a Glycoprotein (GP) Ilb/IIIa receptor antagonist, a P2Y12 receptor antagonist, a Lp-PLA2-inhibitor, a leukotriene inhibitor, a MIF antagonist, or a combination thereof. In some embodiments the cargo moiety is atorvastatin, cerivastatin, fluvastatin, lovastatin, mevastatin, pitavastatin, pravastatin, rosuvastatin, simvastatin, simvastatin and ezetimibe, lovastatin and niacin, extended-release, atorvastatin and amlodipine besylate, simvastatin and niacin, extended-release, bezafibrate, ciprofibrate, clofibrate, gemfibrozil, fenofibrate, DF4 (Ac-D-W-F-K-A-F-Y-D-K-V-A-E-K-F-K-E-A-F-NH2 (SEQ ID NO: 55)), DF5, RVX-208 (Resverlogix), avasimibe, pactimibe sulfate (CS-505), CI-1011 (2,6-diisopropylphenyl [(2, 4,6-triisopropylphenyl)acetyl]sulfamate), CI-976 (2,2-dimethyl-N-(2,4,6-trimethoxyphenyl)dodecanamide), VULM1457 (1-(2,6-diisopropyl-phenyl)-3-[4-(4′-mtrophenylthio)phenyl]urea), CI-976 (2,2-dimethyl-N-(2,4,6-trimethoxyphenyl)dodecanamide), E-5324 (n-butyl-N′-(2-(3-(5-ethyl-4-phenyl-1H-imidazol-1-yl)propoxy)-6-methylphenyl)urea), HL-004 (N-(2,6-diisopropylphenyl) tetradecylthioacetamide), KY-455 (N-(4,6-dimethyl-1-pentylindolin-7-yl)-2,2-dimethylpropanamide), FY-087 (N-[2-[N′-pentyl-(6,6-dimethyl-2,4-heptadiynyl)amino]ethyl]-(2-methyl-1-naphthyl-thio)acetamide), MCC-147 (Mitsubishi Pharma), F 1251 1 ((S)-2′,3′,5′-trimethyl-4′-hydroxy-alpha-dodecylthioacetanihde), SMP-500 (Sumitomo Pharmaceuticals), CL 277082 (2,4-difluoro-phenyl-N[[4-(2,2-dimethylpropyl)phenyl]methyl]-N-(hepthyl)urea), F-1394 ((1s,2s)-2 [3-(2,2-dimethylpropyl)-3-nonylureido]aminocyclohexane-1-yl 3-[N-(2,2,5,5-tetramethyl-1,3-dioxane-4-carbonyl)amino]propionate), CP-113818 (N-(2,4-bis(methylthio)-6-methylpyridm-3-yl)-2-(hexylthio)decanoic acid amide), YM-750, torcetrapib, anacetrapid, JTT-705 (Japan Tobacco/Roche), abciximab, eptifibatide, tirofiban, roxifiban, variabihn, XV 459 (N(3)-(2-(3-(4-formamidinophenyl)isoxazolm-5-yl)acetyl)-N(2)-(1-butyloxycarbonyl)-2,3-diaminopropionate), SR 121566A (3-[N-{4-[4-(aminoiminomethyl)phenyl]-1,3-thiazol-2-yl J-N-(I-carboxymethylpipeπd-4-yl) aminol propionic acid, trihydrochloride), FK419 ((S)-2-acetylamino-3-[(R)-[1-[3-(pipeπdin-4-yl)propionyl]pipeπdm-3-ylcarbonyl]ammo]propionic acid tπhydrate), clopidogrel, prasugrel, cangrelor, AZD6140 (AstraZeneca); MRS 2395 (2,2-Dimethyl-propionic acid 3-(2-chloro-6-methylaminopurin-9-y])-2-(2,2-dimethyl-propionyloxymethyl)-propyl ester); BX 667 (Berlex Biosciences); BX 048 (Berlex Biosciences); darapladib (SB 480848); SB-435495 (GlaxoSmithKline); SB-222657 (GlaxoSmithKline); SB-253514 (GlaxoSmithKline); A-81834 (3-(3-(1,1-dimethylethylthio-5-(quinoline-2-ylmethoxy)-1-(4-chloromethylphenyl)indole-2-yl)-2,2-dimethylpropionaldehyde oxime-O-2-acetic acid; AMEI 03 (Amira); AME803 (Amira); atreleuton; BAY-x-1005 ((R)-(+)-alpha-cyclopentyl-4-(2-quinolinylmethoxy)-Benzeneacetic acid); CJ-13610 (4-(3-(4-(2-Methyl-imidazol-1-yl)-phenylsulfanyl)-phenyl)-tetrahydro-pyran-4-carboxylic acid amide); DG-031 (DeCode); DG-051 (DeCode); MK886 (1-[(4-chlorophenyl)methyl]3-[(1,1-dirnethylethyl)thio]-α,α-dimethyl-5-(1-methylethyl)-1H-indole-2-propanoic acid, sodium salt); MK591 (3-(1-4[(4-chlorophenyl)methyl]-3-[(t-butylthio)-5-((2-quinoly)methoxy)-1H-indole-2]-, dimehtylpropanoic acid); RP64966 ([4-[5-(3-Phenyl-propyl)thiophen-2-yl]butoxy]acetic acid); SA6541 ((R)-S-[[4-(dimethylamino)phenyl]methyl]-N-(3-mercapto-2methyl-1-oxopropyl-L-cycteine); SC-56938 (ethyl-1-[2-[4-(phenylmethyl)phenoxy]ethyl]-4-piperidine-carboxylate); VIA-2291 (Via Pharmaceuticals); WY-47,288 (2-[(1-naphthalenyloxy)methyl]quinoline); zileuton; ZD-2138 (6-((3-fluoro-5-(tetrahydro-4-methoxy-2H-pyran-4yl)phenoxy)methyl)-]-methyl-2(1H)-quinlolinone); or combinations thereof.

In some embodiments, the drug is an agent that modulates death (e.g., via apoptosis or necrosis) of a cell. In some embodiments, the drug is a cytotoxic agent. In some embodiments, the drug is maytansine, methotrexate (RHEUMATREX®, Amethopterin); cyclophosphamide (CYTOXAN®); thalidomide (THALIDOMID®); paclitaxel; pemetrexed; pentostatin; pipobroman; pixantrone; plicamycin; procarbazine; proteasome inhibitors (e.g.; bortezomib); raltitrexed; rebeccamycin; rubitecan; SN-38; salinosporamide A; satraplatin; streptozotocin; swainsonine; tariquidar; taxane; tegafur-uracil; temozolomide; testolactone; thioTEPA; tioguanine; topotecan; trabectedin; tretinoin; triplatin tetranitrate; tris(2-chloroethyl)amine; troxacitabine; uracil mustard; valrubicin; vinblastine; vincristine; vinorelbine; vorinostat; zosuquidar; or a combination thereof. In some embodiments, the drug is a pro-apoptotic agent. In some embodiments, the drug is an anti-apoptotic agent. In some embodiments, the drug is selected from: minocycline; SB-203580 (4-(4-Fluorophenyl)-2-(4-methylsulfinyl phenyl)-5-(4-pyridyl) 1H-imidazole); PD 169316 (4-(4-Fluorophenyl)-2-(4-nitrophenyl)-5-(4-pyridyl)-1H-imidazole); SB 202190 (4-(4-Fluorophenyl)-2-(4-hydroxyphenyl)-5-(4-pyridyl)-1H-imidazole); RWJ 67657 (4-[4-(4-fluorophenyl)-1-(3-phenylpropyl)-5-(4-pyridinyl)-1H-imidazol-2-yl]-3-butyn-1-ol); SB 220025 (5-(2-Amino-4-pyrimidinyl)-4-(4-fluorophenyl)-1-(4-piperidinlyl)imidazole); D-JNKI-I ((D)-hJIP175-157-DPro-DPro-(D)-HIV-TAT57-48); AM-1 1 1 (Auris); SP600125 (anthra[1,9-cd]pyrazol-6(2H)-one); JNK Inhibitor I ((L)-HIV-T AT48-57-PP-JBD20); JNK Inhibitor III ((L)-HIV-T AT47-57-gaba-c-Junδ33-57); AS601245 (1,3-benzothiazol-2-yl (2-[[2-(3-pyridinyl)ethyl]amino]-4 pyrimidinyl) acetonitrile); JNK Inhibitor VI (H2N-RPKRPTTLNLF-NH2 (SEQ ID NO: 56)); JNK Inhibitor VIII (N-(4-Amino-5-cyano-6-ethoxypyridin-2-yl)-2-(2,5-dimethoxyphenyl)acetamide); JNK Inhibitor IX (N-(3-Cyano-4,5,6,7-tetrahydro-1-benzothien-2-yl)-1-naphthamide); dicumarol (3,3′-Methylenebis(4-hydroxycoumarin)); SC-236 (4-[5-(4-chlorophenyl)-3-(trifluoromethyl)-1H-pyrazol-1-yl]benzene-sulfonamide); CEP-1347 (Cephalon); CEP-11004 (Cephalon); an artificial protein comprising at least a portion of a Bcl-2 polypeptide; a recombinant FNK; V5 (also known as Bax inhibitor peptide V5); Bax channel blocker ((±)-1-(3,6-Dibromocarbazol-9-yl)-3-piperazin-1-yl-propan-2-ol); Bax inhibiting peptide P5 (also known as Bax inhibitor peptide P5); Kp7-6; FAIM(S) (Fas apoptosis inhibitory molecule-short); FAIM(L) (Fas apoptosis inhibitory molecule-long); Fas:Fc; FAP-I; NOK2; F2051; F1926; F2928; ZB4; Fas M3 mAb; EGF; 740 Y-P; SC 3036 (KKHTDDGYMPMSPGVA (SEQ ID NO: 57)); PI 3-kinase Activator (Santa Cruz Biotechnology, Inc.); Pam3Cys ((S)-(2,3-bis(palmitoyloxy)-(2RS)-propyl)-N-palmitoyl-(R)-Cys-(S)-Ser(S)-Lys4-OH, trihydrochloride); Actl (NF-κB activator 1); an anti-IκB antibody; Acetyl-1 1-keto-b-Boswellic Acid; Andrographolide; Caffeic Acid Phenethyl Ester (CAPE); Gliotoxin; Isohelenin; NEMO-Binding Domain Binding Peptide (DRQIKIWFQNRRMKWKKTALDWSWLQTE (SEQ ID NO: 58)); NF-κB Activation Inhibitor (6-Amino-4-(4-phenoxyphenylethylamino)quinazoline); NF-κB Activation Inhibitor II (4-Methyl-N1-(3-phenylpropyl)benzene-1,2-diamine); NF-κB Activation Inhibitor III (3-Chloro-4-nitro-N-(5-nitro-2-thiazolyl)-benzamide); NF-κB Activation Inhibitor IV ((E)-2-Fluoro-4′-methoxystilbene); NF-κB Activation Inhibitor V (5-Hydroxy-(2,6-diisopropylphenyl)-1H-isoindole-1,3-dione); NF-κB SN50 (AAVALLPAVLLALLAPVQRKRQKLMP (SEQ ID NO: 59)); Oridonin; Parthenolide; PPM-18 (2-Benzoylamino-1,4-naphthoquinone); RoI 06-9920; Sulfasalazine; TIRAP Inhibitor Peptide (RQIKIWFNRRMKWKKLQLRDAAPGGAIVS (SEQ ID NO: 60)); Withaferin A; Wogonin; BAY 1 1-7082 ((E)3-[(4-Methylphenyl)sulfonyl]-2-propenenitrile); BAY 1 1-7085 ((E)3-[(4-t-Butylphenyl)sulfonyl]-2-propenenitrile); (E)-Capsaicin; Aurothiomalate (ATM or AuTM); Evodiamine; Hypoestoxide; IKK Inhibitor m (BMS-345541); IKK Inhibitor VII; IKK Inhibitor X; IKK Inhibitor II; IKK-2 Inhibitor IV; IKK-2 Inhibitor V; IKK-2 Inhibitor VI; IKK-2 Inhibitor (SC-514); IκB Kinase Inhibitor Peptide; IKK-3 Inhibitor IX; ARRY-797 (Array BioPharma); SB-220025 (5-(2-Amino-4-pyrimidinyl)-4-(4-fluorophenyl)-1-(4-piperidinlyl)imidazole); SB-239063 (trans-4-[4-(4-Fluorophenyl)-5-(2-methoxy-4-pyrimidinyl)-1H-imidazol-1-yl]cyclohexanol); SB-202190 (4-(4-Fluorophenyl)-2-(4-hydroxyphenyl)-5-(4-pyridyl)1H-imidazole); JX-401 (-[2-Methoxy-4-(methylthio)benzoyl]-4-(phenylmethyl)piperidine); PD-169316 (4-(4-Fluorophenyl)-2-(4-nitrophenyl)-5-(4-pyridyl)-1H-imidazole); SKF-86002 (6-(4-Fluorophenyl)-2,3-dihydro-5-(4-pyridinyl)imidazo[2,1-b]thiazole dihydrochloride); SB-200646 (N-(I-Methyl-1H-indol-5-yl)-N′-3-pyridinylurea); CMPD-I (2′-Fluoro-N-(4-hydroxyphenyl)-[1, 1′-biphenyl]-4-butanamide); EO-1428 ((2-Methylphenyl)-[4-[(2-amino-4-bromophenyl)amino]-2-chlorophenyl]methanone); SB-253080 (4-[5-(4-Fluorophenyl)-2-[4-(methylsulfonyl)phenyl]-1H-imidazol-4-yl]pyridine); SD-169 (1H-Indole-5-carboxamide); SB-203580 (4-(4-Fluorophenyl)-2-(4-methylsulfinyl phenyl)-5-(4-pyridyl) 1H-imidazole); TZP-101 (Tranzyme Pharma); TZP-102 (Tranzyme Pharma); GHRP-6 (growth hormone-releasing peptide-6); GHRP-2 (growth hormone-releasing peptide-2); EX-1314 (Elixir Pharmaceuticals); MK-677 (Merck); L-692,429 (Butanamide, 3-amino-3-methyl-N-(2,3,4,5-tetrahydro-2-oxo-l-((2′-(1H-tetrazol-5-yl)(1, 1′-biphenyl)-4-yl)methyl)-1 H-1-benzazepin-3-yl)-, (R)-); EP1 572 (Aib-DTrp-DgTrp-CHO); diltiazem; metabolites of diltiazem; BRE (Brain and Reproductive organ-Expressed protein); verapamil; nimodipine; diltiazem; omega-conotoxin; GVIA; amlodipine; felodipine; lacidipine; mibefradil; NPPB (5-Nitro-2-(3-phenylpropylamino)benzoic Acid); flunarizine; erythropoietin; piperine; hemin; brazilin; z-V AD-FMK (Benzyloxycarbonyl-Val-Ala-Asp(OMe)-fluoromethylketone); z-LEHD-FMK (SEQ ID NO: 61) (benzyloxycarbonyl-Leu-Glu(OMe)-His-Asp(OMe)-fluoromethylketone); B-D-FMK (boc-aspartyl(Ome)-fluoromethylketone); Ac-LEHD-CHO (SEQ ID NO: 62) (N-acetyl-Leu-Glu-His-Asp-CHO); Ac-IETD-CHO (SEQ ID NO: 63) (N-acetyl-Ile-Glu-Thr-Asp-CHO); z-IETD-FMK (SEQ ID NO: 64) (benzyloxycarbonyl-Ile-Glu(OMe)-Thr-Asp(OMe)-fluoromethy Iketone); FAM-LEHD-FMK (SEQ ID NO: 65) (benzyloxycarbonyl Leu-Glu-His-Asp-fluoromethyl ketone); FAM-LETD-FMK (SEQ ID NO: 66) (benzyloxycarbonyl Leu-Glu-Thr-Asp-iluoromethyl ketone); Q-VD-OPH (Quinoline-Val-Asp-CH2-O-Ph); XIAP; cIAP-1; cIAP-2; ML-IAP; ILP-2; NAIP; Survivin; Bruce; IAPL-3; fortilin; leupeptine; PD-150606 (3-(4-Iodophenyl)-2-mercapto-(Z)-2-propenoic acid); MDL-28170 (Z-Val-Phe-CHO); calpeptin; acetyl-calpastatin; MG 132 (N-t(phenylmethoxy)carbonyl]-L-leucyl-N-[(1S)-1-formyl-3-methylbutyl]-L-leucinamide); MYODUR; BN 82270 (Ipsen); BN 2204 (Ipsen); AHLi-11 (Quark Pharmaceuticals), an mdm2 protein, pifithrin-α (1-(4-Methylphenyl)-2-(4,5,6,7-tetrahydro-2-imino-3(2H)-benzothiazolyl)ethanone); trans-stilbene; cis-stilbene; resveratrol; piceatannol; rhapontin; deoxyrhapontin; butein; chalcon; isoliquirtigen; butein; 4,2′,4′-trihydroxychalcone; 3,4,2′,4′,6′-pentahydroxychalcone; flavone; morin; fisetin; luteolin; quercetin; kaempferol; apigenin; gossypetin; myricetin; 6-hydroxyapigenin; 5-hydroxyflavone; 5,7,3′,4′,5′-pentahydroxyflavone; 3,7,3′,4′,5′-pentahydroxyflavone; 3,6,3′,4′-tetrahydroxyflavone; 7,3′,4′,5′-tetrahydroxyflavone; 3,6,2′,4′-tetrahydroxyflavone; 7,4′-dihydroxyflavone; 7,8,3′,4′-tetrahydroxyflavone; 3,6,2′,3′-tetrahydroxyflavone; 4′-hydroxyflavone; 5-hydroxyflavone; 5,4′-dihydroxyflavone; 5,7-dihydroxyflavone; daidzein; genistein; naringenin; flavanone; 3,5,7,3′,4′-pentahydroxyflavanone; pelargonidin chloride; cyanidin chloride; delphinidin chloride; (−)-epicatechin (Hydroxy Sites: 3,5,7,3′,4′); (−)-catechin (Hydroxy Sites: 3,5,7,3′,4′); (−)-gallocatechin (Hydroxy Sites: 3,5,7,3′,4′,5′) (+)-catechin (Hydroxy Sites: 3,5,7,3′,4′); (+)-epicatechin (Hydroxy Sites: 3,5,7,3′,4′); Hinokitiol (b-Thujaplicin; 2-hydroxy-4-isopropyl-2,4,6-cycloheptatrien-1-one); L-(+)-Ergothioneine ((S)-a-Carboxy-2,3-dihydro-N,N,N-trimethyl-2-thioxo-1H-imidazole4-ethanaminium inner salt); Caffeic Acid Phenyl Ester; MCI-186 (3-Methyl-1-phenyl-2-pyrazolin-5-one); HBED (N,N′-Di-(2-hydroxybenzyl)ethylenediamine-N,N′-diacetic acid*H20); Ambroxol (trans-4-(2-Amino-3,5-dibromobenzylamino)cyclohexane-HCl; and U-83836E ((−)-2-((4-(2,6-di-1-Pyrrolidinyl-4-pyrimidinyl)-1-piperzainyl)methyl)-3,4-dihydro-2,5,7,8-tetramethyl-2H-1-benzopyran-6-ol*2HCl); β-1′-5-methyl-nicotinamide-2′-deoxyribose; β-D-r-5-methyl-nico-tinamide-2′-deoxyribofuranoside; β-1′-4,5-dimethyl-nicotinamide-2′-de-oxyribose; β-D-1′-4,5-dimethyl-nicotinamide-2′-deoxyribofuranoside; 1-Naphthyl PPI (1-(1,1-Dimethylethyl)-3-(1-naphthalenyl)-1H-pyrazolo[3,4-d]pyrimidin-4-amine); Lavendustin A (5-[[(2,5-Dihydroxyphenyl)methyl][(2-hydroxyphenyl)methy 1]amino]-2-hydroxybenzoic acid); MNS (3,4-Methylenedioxy-b-nitrostyrene), PPI (1-(1,1-Dimethylethyl)-1-(4-methylphenyl)-1H-pyrazolo[3, 4-d]pyπmidin-4-amine), PP2 (3-(4-chlorophenyl) 1-(1, 1-dimethylethyl)-l H-pyrazolo[3,4-d]pyπmidin-4-amine), KX-004 (Kinex), KX-005 (Kinex), KX-136 (Kinex), KX-174 (Kinex), KX-141 (Kinex), KX2-328 (Kinex), KX-306 (Kinex), KX-329 (Kinex), KX2-391 (Kinex), KX2-377 (Kinex), ZD4190 (Astra Zeneca, N-(4-bromo-2-fluorophenyl)-6-methoxy-7-(2-(1H-1,2,3-triazol-1-yl)ethoxy)quinazolin-4-amine), AP22408 (Airad Pharmaceuticals), AP23236 (Aπad Pharmaceuticals), AP23451 (Atad Pharmaceuticals), AP23464 (Atad Pharmaceuticals), AZD0530 (Astra Zeneca), AZM475271 (M475271, Astra Zeneca), Dasatmib (N-(2-chloro-6-methylphneyl)-2-(6-(4-(2-hydroxyethyl)-piperazin-1-yl)-2-methylpynmidin-4-yl)ammo) thiazole-5-carboxamide), GN963 (trans-4-(6,7-dimethoxyqmnoxalm-2ylamino)cyclohexanol sulfate); Bosutimb (4-((2,4-dichloro-5-methoxyphenyl)ammo)-6-methoxy-7-(3-(4-methyl-1-prperazmyl)propoxy)-3-quinolinecarboniπle), or combinations thereof.

In some embodiments, a MTS molecule disclosed herein has the formula (A-X-B)n-L, wherein L is a lipid, A is a peptide with a sequence comprising 5 to 9 consecutive acidic ammo acids, wherein the amino acids are selected from aspartates and glutamates, B is a peptide with a sequence comprising 5 to 20 consecutive basic amino acids, X is a linker, and n is an integer between 1 and 20, and wherein L is bound to an (A-X-B) moiety by a bond with a B.

In some embodiments, the lipid entraps a hydrophobic molecule In some embodiments, the lipid entraps at least one agent selected from the group consisting of a therapeutic moiety or an imaging moiety.

In some embodiments, the lipid is PEGylated In some embodiments, the lipid is PEG(2K)-phosphatidylethanolamine.

In some embodiments, a MTS molecule disclosed herein has the formula (A-X-B-C)n-M, wherein C is a cargo moiety; A is a peptide with a sequence comprising 5 to 9 consecutive acidic amino acids, wherein the amino acids are selected from: aspartates and glutamates; B is a peptide with a sequence comprising 5 to 20 consecutive basic amino acids; X is a linker that is cleavable by thrombin; M is a macromolecular carrier; and n is an integer between 1 and 20.

Disclosed herein, in certain embodiments, is a MTS molecule with increased in vivo circulation In some embodiments, a MTS molecule disclosed herein has the formula (A-X-B-C)n-M, wherein C is a cargo moiety, A is a peptide with a sequence comprising 5 to 9 consecutive acidic ammo acids, wherein the amino acids are selected from aspartates and glutamates, B is a peptide with a sequence comprising 5 to 20 consecutive basic amino acids; X is a linker; M is a macromolecular carrier; and n is an integer between 1 and 20.

The term “carrier” indicates an inert molecule that increases (a) plasma half-life and (b) solubility. In some embodiments, a carrier decreases uptake of a MTS molecule into cartilage. In some embodiments, a carrier decreases uptake of a MTS molecule into joints. In some embodiments, a carrier decreases uptake of a MTS molecule into the liver. In some embodiments, a carrier decreases uptake of a MTS molecule into kidneys.

In some embodiments, a carrier increases plasma half-life and solubility by reducing glomerular filtration. In some embodiments, a carrier increases tumor uptake due to enhanced permeability and retention (EPR) of tumor vasculature.

In some embodiments, M is bound to A. In some embodiments, M is bound to A at the n-terminal poly glutamate. In some embodiments, M is bound to A (or, the n-terminal poly glutamate) by a covalent linkage. In some embodiments, the covalent linkage comprises an ether bond, thioether bond, amine bond, amide bond, carbon-carbon bond, carbon-nitrogen bond, carbon-oxygen bond, or carbon-sulfur bond.

In some embodiments, M is bound to B. In some embodiments, M is bound to B at the c-terminal polyarginine. In some embodiments, M is bound to B (or, the c-terminal polyarginine) by a covalent linkage. In some embodiments, the covalent linkage comprises an ether bond, thioether bond, amine bond, amide bond, carbon-carbon bond, carbon-nitrogen bond, carbon-oxygen bond, or carbon-sulfur bond.

In some embodiments, M is selected from a protein, a synthetic or natural polymer, or a dendrimer. In some embodiments, M is selected from dextran, a PEG polymer (e.g., PEG 5 kDa and PEG 12 kDa), albumin, or a combination thereof. In some embodiments, M is a PEG polymer.

In some embodiments, the size of the carrier is between 50 kDa and 70 kDa. In some embodiments, small amounts of negative charge keep peptides out of the liver while not causing synovial uptake.

In some embodiments, the MTS molecule is conjugated to albumin. In certain instances, albumin is excluded from the glomerular filtrate under normal physiological conditions. In some embodiments, the MTS molecule comprises a reactive group such as maleimide that can form a covalent conjugate with albumin. A MTS molecule comprising albumin results in enhanced accumulation of cleaved MTS molecules in tumors in a cleavage dependent manner. See, Example 2. In some embodiments, albumin conjugates have good pharmacokinetic properties but are difficult to work with synthetically.

In some embodiments, the MTS molecule is conjugated to a PEG polymer. In some embodiments, the MTS molecule is conjugated to a PEG 5 kDa polymer. In some embodiments, the MTS molecule is conjugated to a PEG 12 kDa polymer. In some embodiments, 5kD PEG conjugates behaved similarly to free peptides. In some embodiments, 12kD PEG conjugates had a longer halflife as compared to free peptides.

In some embodiments, the MTS molecule is conjugated to a dextran. In some embodiments, the MTS molecule is conjugated to a 70 kDa dextran. In some embodiments, dextran conjugates, being a mixture of molecular weights, are difficult to synthesize and purify reproducibly.

In some embodiments, the MTS molecule is conjugated to streptavidin.

In some embodiments, the MTS molecule is conjugated to a fifth generation PAMAM dendrimer.

In some embodiments, a carrier is capped. See Example 1 for methods of capping. In some embodiments, capping a carrier improves the pharmacokinetics and reduces cytotoxicity of a carrier by adding hydrophilicity. In some embodiments, the cap is selected from: Acetyl, succinyl, 3-hydroxypropionyl, 2-sulfobenzoyl, glycidyl, PEG-2, PEG-4, PEG-8 and PEG-12.

In some embodiments, a MTS molecule disclosed herein has the formula (A-X-B)n-D, wherein D is a dendrimer; A is a peptide with a sequence comprising 5 to 9 consecutive acidic amino acids, wherein the amino acids are selected from: aspartates and glutamates; B is a peptide with a sequence comprising 5 to 20 consecutive basic amino acids; X is a linker; and n is an integer between 1 and 20; and wherein D is bound to an (A-X-B) moiety by a bond with a B. In some embodiments, D is bound to an (A-X-B) moiety by a bond with a polyarginine terminus. In some embodiments, D comprises at least one cargo moiety.

As used herein, “dendrimer” means a poly-functional (or, poly-branched) molecule. In some embodiments, a denrimer is a structure in which a central molecule branches repetitively and repetitiously. In some embodiments, the dendrimer is a nanoparticle.

In some embodiments, D is bound to B (or, the c-terminal polyarginine) by a covalent linkage. In some embodiments, the covalent linkage comprises an ether bond, thioether bond, amine bond, amide bond, carbon-carbon bond, carbon-nitrogen bond, carbon-oxygen bond, or carbon-sulfur bond.

In some embodiments, a plurality of (A-X-B) moieties are attached to D. See, Example 3. In some embodiments, a plurality of cargo moieties are attached to D. In some embodiments, (a) a plurality of (A-X-B) moieties are attached to D; and (b) a plurality of cargo moieties are attached to D.

In some embodiments, the dendrimer comprises a reactive group such as maleimide that can form a covalent conjugate with albumin. In some embodiments, a dendrimer is conjugated to a MTS molecule via a maleimide linker at the C-terminal end of the MTS molecule.

In some embodiments, conjugating a MTS molecule to a dendrimer increases plasma half-life as compared to an unconjugated (or, free) MTS molecule. In some embodiments, a MTS molecule conjugated to a dendrimer results in a decrease in acute toxicity as compared to unconjugated MTS molecules. In some embodiments, a MTS molecule conjugated to a dendrimer reduces uptake by synovium, cartilage and kidney as compared to unconjugated MTS molecules.

In some embodiments, a MTS molecule conjugated to a dendrimeric nanoparticle is used to target tumor associated macrophages. In some embodiments, a MTS molecule conjugated to a dendrimeric nanoparticle, wherein the nanoparticle further comprises Ricin A, is used to poison subcutaneous macrophages.

MTS molecules having features of the invention may be synthesized by standard synthetic techniques, such as, for example, solid phase synthesis including solid phase peptide synthesis. An example of peptide synthesis using Fmoc is given as Example 1 below in WO 2005/042034). For example, conventional solid phase methods for synthesizing peptides may start with N-alpha-protected amino acid anhydrides that are prepared in crystallized form or prepared freshly in solution, and are used for successive amino acid addition at the N-terminus. At each residue addition, the growing peptide (on a solid support) is acid treated to remove the N-alpha-protective group, washed several times to remove residual acid and to promote accessibility of the peptide terminus to the reaction medium. The peptide is then reacted with an activated N-protected amino acid symmetrical anhydride, and the solid support is washed. At each residue-addition step, the amino acid addition reaction may be repeated for a total of two or three separate addition reactions, to increase the percent of growing peptide molecules which are reacted. Typically, 1 to 2 reaction cycles are used for the first twelve residue additions, and 2 to 3 reaction cycles for the remaining residues.

After completing the growing peptide chains, the protected peptide resin is treated with a strong acid such as liquid hydrofluoric acid or trifluoroacetic acid to deblock and release the peptides from the support. For preparing an amidated peptide, the resin support used in the synthesis is selected to supply a C-terminal amide, after peptide cleavage from the resin. After removal of the strong acid, the peptide may be extracted into 1M acetic acid solution and lyophilized. The peptide can be isolated by an initial separation by gel filtration, to remove peptide dimers and higher molecular weight polymers, and also to remove undesired salts The partially purified peptide may be further purified by preparative HPLC chromatography, and the purity and identity of the peptide confirmed by amino acid composition analysis, mass spectrometry and by analytical HPLC (e.g., in two different solvent systems).

The invention also provides polynucleotides encoding MTS molecules described herein. The term “polynucleotide” refers to a polymeric form of nucleotides of at least 10 bases in length. The nucleotides can be ribonucleotides, deoxynucleotides, or modified forms of either type of nucleotide. The term includes single and double stranded forms of DNA. The term therefore includes, for example, a recombinant DNA which is incorporated into a vector, e.g., an expression vector; into an autonomously replicating plasmid or virus; or into the genomic DNA of a prokaryote or eukaryote, or which exists as a separate molecule (e.g., a cDNA) independent of other sequences.

These polynucleotides include DNA, cDNA, and RNA sequences which encode MTS molecules having features of the invention, or portions thereof. Peptide portions may be produced by recombinant means, including synthesis by polynucleotides encoding the desired amino acid sequence. Such polynucleotides may also include promoter and other sequences, and may be incorporated into a vector for transfection (which may be stable or transient) in a host cell.

The construction of expression vectors and the expression of genes in transfected cells involves the use of molecular cloning techniques that are well known in the art. See, for example, Sambrook et al., Molecular Cloning—A Laboratory Manual, Cold Spring Harbor Laboratory, Cold Spring Harbor, N.Y., (1989) and Current Protocols in Molecular Biology, F. M. Ausubel et al., eds., (Current Protocols, a joint venture between Greene Publishing Associates, Inc. and John Wiley & Sons, Inc., most recent Supplement). Nucleic acids used to transfect cells with sequences coding for expression of the polypeptide of interest generally will be in the form of an expression vector including expression control sequences operatively linked to a nucleotide sequence coding for expression of the polypeptide. As used herein, “operatively linked” refers to a juxtaposition wherein the components so described are in a relationship permitting them to function in their intended manner. A control sequence operatively linked to a coding sequence is ligated such that expression of the coding sequence is achieved under conditions compatible with the control sequences. “Control sequence” refers to polynucleotide sequences which are necessary to effect the expression of coding and non-coding sequences to which they are ligated. Control sequences generally include promoter, ribosomal binding site, and transcription termination sequence. The term “control sequences” is intended to include, at a minimum, components whose presence can influence expression, and can also include additional components whose presence is advantageous, for example, leader sequences and fusion partner sequences. As used herein, the term “nucleotide sequence coding for expression of a polypeptide refers to a sequence that, upon transcription and translation of mRNA, produces the polypeptide. This can include sequences containing, e.g., introns. As used herein, the term “expression control sequences” refers to nucleic acid sequences that regulate the expression of a nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (i.e., ATG) in front of a protein-encoding gene, splicing signals for introns, maintenance of the correct reading frame of that gene to permit proper translation of the mRNA, and stop codons.

Any suitable method is used to construct expression vectors containing the fluorescent indicator coding sequence and appropriate transcriptional/translational control signals. Any methods which are well known to those skilled in the art can be used to construct expression vectors containing the fluorescent indicator coding sequence and appropriate transcriptional/translational control signals. These methods include in vitro recombinant DNA techniques, synthetic techniques and in vivo recombination/genetic recombination. (See, for example, the techniques described in Maniatis, et al., Molecular Cloning A Laboratory Manual, Cold Spring Harbor Laboratory, N. Y., 1989). Transformation of a host cell with recombinant DNA may be carried out by conventional techniques as are well known to those skilled in the art.

Where the host is prokaryotic, such as E. coli, competent cells which are capable of DNA uptake can be prepared from cells harvested after exponential growth phase and subsequently treated by the CaCl₂ method by procedures well known in the art. Alternatively, MgCl₂ or RbCl can be used. Transformation can also be performed after forming a protoplast of the host cell or by electroporation.

When the host is a eukaryote, such methods of transfection of DNA as calcium phosphate co-precipitates, conventional mechanical procedures such as microinjection, electroporation, insertion of a plasmid encased in liposomes, or virus vectors may be used. Eukaryotic cells can also be cotransfected with DNA sequences encoding the fusion polypeptide of the invention, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein. (Eukagotic Viral Vectors, Cold Spring Harbor Laboratory, Gluzman ed., 1982). Techniques for the isolation and purification of polypeptides of the invention expressed in prokaryotes or eukaryotes may be by any conventional means such as, for example, preparative chromatographic separations and immunological separations such as those involving the use of monoclonal or polyclonal antibodies or antigen.

It will be understood that the compounds of the present invention can be formulated in pharmaceutically and or diagnostically useful compositions. Such pharmaceutical and diagnositcally useful compositions may be prepared according to well known methods. For example, MTS compounds having features of the invention, and having a cargo portion C that is, for example, a therapeutic moiety or a detection moiety, may be combined in admixture with a pharmaceutically acceptable carrier vehicle or a diagnostic buffering agent. Suitable vehicles and agents and their formulation, inclusive of other human proteins, e.g. human serum albumin are described, for example, in Remington's Pharmaceutical Sciences by E. W. Martin and, the techniques described in Maniatis, et al., Molecular Cloning A Laboratory Manual, Cold Spring Harbor Laboratory, N. Y., 1989-2013, which are hereby incorporated by reference. Such compositions will contain an effective amount of the compounds hereof together with a suitable amount of vehicle in order to prepare pharmaceutically acceptable compositions suitable for effective administration. Dosages and dosing regimens may be determined for the indications and compounds by methods known in the art, including determining (e.g., in experimental animals) the effective dose which causes half of those treated to respond to the treatment (ED₅₀) by providing a range of doses to experimental animals or subjects and noting the responses.

### EXAMPLES

### Example 1

### Methods for Measuring Ex Vivo Cleavage of FRET-Based Enzymatically Cleavable Peptide Probes by Tumor Extract

The rationale is to identify enzymatically positive tumors in a patient population that may benefit from the use of enzymatically cleavable peptide probes for early tumor detection and intraoperative margin evaluation. Experiments have been performed using probes that detected either MMPs (PLGLAG (SEQ ID NO: 1) and PLGC(met)AG (SEQ ID NO: 2) or elastases (RLQLK(acetyl)L (SEQ ID NO: 26). A panel of probes will be expanded to include other tumor expressed proteases.

Xenograft Tumor Extracts:

Animals models of a variety of cancers cancer were generated as previously described. Tumors were grown to 0.5 cm-1 cm and then surgically excised. Following dissection, tissue was gently homogenized in PBS, while kept cooled on ice to minimize the release of intracellular and intraorganellar proteases. Homogenates were microcentrifuged at 14,000×g for 1 minute, and supernatants (extracts) were tested for ability to increase the Cy5 donor:Cy7 acceptor emission ratio of a FRET-based ACPP. The effect of freezing the tumor specimen before homogenization was tested and showed that the assay can be reliably performed on frozen material, simplifying the logistics of collection. Furthermore, banked clinical specimens may be retrospectively analyzable and able to be compared with known outcomes.

Surgical Specimen Extracts:

Surgical specimens from patients undergoing excision of neoplasms were obtained under University of California, San Diego (UCSD) IRB approval. Following receipt of tumor specimens from the pathology department, tissue was frozen in −80C for varying periods of time from 1 day to 1 year. Frozen specimens are thawed and assayed as described in the previous subsection (Xenograft tumor extracts).

Peptide Synthesis:

FRET-based ACPPs were synthesized by attaching a fluorescent acceptor, Cy7, to the polyanionic domain of the previously described ACPP2, 3 so that the donor (Cy5) and acceptor (Cy7) fluorophores in the uncleaved probe are sufficiently close to each other for FRET to occur This FRET quenches the Cy5 emission intensity and causes re-emission of Cy7. Upon cleavage of the ACPP linker, FRET was disrupted and Cy5 emission is increased while Cy7 re-emission is eliminated. This loss of FRET, imaged in vivo by multispectral imaging in animal tumor models, produced faster and more intense tumor:background contrast than occurred with previously published non-FRET ACPPs.

Ex Vivo Cleavage:

Ex-vivo cleavages were done with 5 μM peptide in 20 μl of PBS containing 1 μM ZnCl2 plus 2-20 μl of 20% tissue extract (20 mg of tissue homogenate per 100 μl), followed by incubation at 37° C. Cleavage of probe will be determined as a ratio of Cy5:Cy7 fluorescence, using either a microplate fluorometer (Tecan M1000) or multispectral imaging (Maestro, CRI) at various time points for different tumor extract concentrations. Cleavage rate will be determined by plotting Cy5:Cy7 ratio as a function of time following addition of tumor extract to the peptide.

In Vivo Imaging:

Human-derived tongue SCC cell lines (named above) or minced tumor specimens from patients will be implanted subcutaneously into nude animals. Tumors were grown to 0.5 cm-1 cm. Animals were injected intravenously with 10 nmoles of FRET-ACPP. Multispectral imaging was performed using Maestro (CRI), and ratio of Cy5:Cy7 fluorescence was measured at 2 hours. Ratio of Cy5:Cy7 fluorescence were measured for tumor and adjacent non-tumor tissue.

ACPP Cleavage Vs. ACPP Fluorescence Uptake Comparison:

Ratio of Cy5:Cy7 fluorescence were measured for tumor and adjacent non-tumor tissue for a given tumor specimen (SCC cell lines or individual human surgical specimens) and were compared with the ex-vivo cleavage rate.

### Example 2

### Generation of Panel of ACPP for Profiling Tumor Protease Activity

Multiple ACPPs with varied protease cleavage sequence will be used to establish each specific tumors protease profile. Currently the panel consist of ACPPs that are selective for MMPs, elastases, plasmin, thrombin. MMP 2,9 cleavable sequence PLGLAG (SEQ ID NO: 1), PLGC(met)AG (SEQ ID NO: 2). Other MMP selective substrates could include RS-(Cit)-G-(homoF)-YLY (SEQ ID NO: 4), CRPAHLRDSG (SEQ ID NO: 5), SLAYYTA (SEQ ID NO: 6), NISDLTAG (SEQ ID NO: 7), PPSSLRVT (SEQ ID NO: 8), SGESLSNLTA (SEQ ID NO: 9), RIGFLR (SEQ ID NO: 10). Elastase cleavable sequence RLQLA(acetyl)L (SEQ ID NO: 11). Plasmin selective substrate RLQLKL (SEQ ID NO: 12). Thrombin selective substrates DPRSFL (SEQ ID NO: 13), PPRSFL (SEQ ID NO: 14), Norleucine-TPRSFL (SEQ ID NO: 15). Chymase selective substrate GVAY|SGA (SEQ ID NO: 16). Urokinase-type plasminogen activator (uPA) and tissue plasminogen activator (tPA) selective substrate YGRAAA (SEQ ID NO: 17). uPA selective sequence YGPRNR (SEQ ID NO: 18).

### Example 3

### Personalized Protease Assay

A personalized protease (PePA) assay will be provided which will find use in a variety of situations. A PePA assay will provide an assay for use in understanding the heterogeneity in levels of tumor specific enzymes between patients for a given cancer diagnosis. A PePA assay will also provide an assay for use in correlating tumor specific enzyme activity with known histologic grade/stage and patient prognosis. A PePA assay will provide an assay for use in identifying which patients may benefit from the use of individual enzymatically activatable probes for staging, medical and surgical management in a variety of cancers.

### Example 4

### Abstract

### Objective:

1. Obtain matrix-metalloproteinase(MMP) expression profiles for head and neck squamous cell carcinoma(HNSCC) specimens from The Cancer Genomic Atlas (TCGA)
2. Demonstrate HNSCC imaging using MMP-cleavable, fluorescently-labeled ratiometric activatable cell-penetrating peptide (RACPP).

### Study Design:

Retrospective human cohort study; prospective animal study

### Setting:

Translational Research Laboratory

### Subjects and Methods:

Patient clinical data and mRNA expression levels of MMP genes were downloaded from TCGA data portal. RACPP provides complementary ratiometric fluorescent contrast (increased Cy5 and decreased Cy7 intensities) when cleaved by MMP2/9. HNSCC-tumor bearing mice were imaged in-vivo after RACPP injection. Histology was evaluated by a pathologist blinded to experimental conditions. Zymography confirmed MMP-2/9 activity in xenografts. RACPP was applied to homogenized human HNSCC specimens and ratiometric fluorescent signal was measured on a microplate reader for ex-vivo analysis.

### Results:

Expression of multiple MMPs including MMP2/9 is greater in patient HNSCC tumors than matched control tissue. In patients with human papilloma virus positive (HPV+) tumors, higher MMP2 and MMP14 expression correlates with worse 5-year survival. Orthotopic tongue HNSCC xenografts showed excellent ratiometric fluorescent labeling with MMP2/9-cleavable RACPP(sensitivity=95.4%, specificity=95.0%). Fluorescence ratios were greater in areas of higher tumor burden(p<0.03), which is useful for intraoperative margin assessment. Ex-vivo, human HNSCC specimens showed greater cleavage of RACPP when compared to control tissue(p=0.009).

### Conclusions:

Human HNSCC tumors show increased mRNA expression of multiple MMPs including MMP2/9. RACPP, a ratiometric fluorescence assay of MMP2/9 activity, was used to show improved occult tumor identification and margin clearance. Ex-vivo assays using RACPP in biopsy specimens may identify patients who will benefit from intraoperative RACPP use.

### Introduction

Head and neck squamous cell carcinoma (HNSCC) is the sixth most common cancer worldwide with an estimated annual burden of 355,000 deaths and 633,000 incident cases¹. Major risk factors include smoking, alcohol abuse, and human papilloma virus (HPV)². Surgical management is usually the primary therapy for this disease, although radiation and chemotherapy also have prominent roles³.

For HNSCC, MMP expression has been shown to have prognostic value⁴⁻⁸. Of the various MMPs thought to be involved in cancer, attention has focused on MMPs 2 and 9 because they are overexpressed in a variety of malignant tumors and their expression is often associated with tumor grade and poor patient prognosis. Absolute levels of MMP2/9 have been used to differentiate between benign papillomas and carcinoma of the larynx⁴. Increased MMP2/9 expression has also been shown to correlate with cancer grade⁵ and decreased survival^{6,7}. In carcinoma of the tongue, increased MMP2/9 expression has been shown to correlate with an increased incidence of lymph node metastases⁸.

In this example, MMP mRNA levels in HNSCC were evaluated using The Cancer Genomic Atlas (TCGA), the largest available collection of HNSCC specimens. The prognostic value of MMP mRNA levels in patients with HPV+ and HPV−HNSCC tumors was evaluated.

Although MMP expression (mRNA and protein) has been associated with tumor grade and poor patient prognosis for a variety of cancers, at the tissue level, MMP activity is regulated by a variety of factors including activation from pro-enzyme form and presence or absence of inhibitors⁹. Consequently MMP activity, rather than expression, may have closer association with tumor biological behavior and therefore greater prognostic value. Activatable cell penetrating peptides (ACPPs), which rely on tumor-associated proteases MMP2/9 to unmask the adhesiveness of CPPs have been previously described^{10,11}. A ratiometric version of ACPPs (RACPPS) which employs Cy5 as a far-red fluorescent resonance energy transfer (FRET) donor and Cy7 as near-infrared FRET acceptor has been recently described. The Cy5 emission is absorbed by Cy7 and re-emitted as near-infrared fluorescence until the intervening linker is cleaved by tumor-associated MMP2/9. This cleavage event increases Cy5:Cy7 emission ratio up to 40-fold and enables tissue retention of the Cy5 fragment¹². ACPP was previously used to improve tumor margin (defined as tumor cells present at the cut edge of the surgical specimen) detection in animal model of melanoma and breast cancer¹³.

In HNSCC, positive margins have been associated with increased local recurrence and a poor prognosis¹⁴. For the majority of solid tumors, salvage surgery or adjuvant therapy not only cause extra trauma and expense but also often fail to remediate the poor outcome¹⁴⁻²⁰. The reason for this observation is likely multifactorial and related in part to the difficulty in identifying the residual cancer during repeat surgery. Therefore, development of more sensitive imaging for accurate detection of positive surgical margins during the primary operation would be one of the most effective means to prevent positive margins, thereby minimizing patient suffering and expense, while improving outcomes.

Using RACPP, MMP2/9 activity levels were compared between patient derived ex-vivo HNSCC specimens versus non-tumor tissue. The use of intravenously applied RACPPs to distinguish between orthotopic HNSCC xenografts from normal tissue and stratify tumor burden at the surgical margin in mice was evaluated.

### Methods

All animal studies were approved by the UCSD Institutional Animal Care and Use Committee. All studies involving tumor samples obtained from HNSCC patients were approved by the UCSD Institutional Review Board.

### The Cancer Genomic Atlas (TCGA)

All available clinical and RNA expression data were downloaded from the TCGA data-portal on Dec. 15, 2013. HPV status was obtained from the TCGA HNSCC working group. HPV status was extracted from sequencing data or RNA data²¹. For tumor-normal comparison, 37 patients (out of 377 total) with matched tumor/normal tissue were considered and paired tests were used.

### Ex-Vivo Assay on HNSCC

Tumor samples were obtained from patients undergoing surgery for mucosal head and neck squamous cell carcinoma and stored at −80° C. until analysis. Samples were homogenized using equal quantities of beads and tissue and twice the volume of PBS. 150 nmol of RACPP was added to 100-175 μl of PBS containing 25 μl of 10% tissue extract. Cleavage of the probe was determined by capturing the Cy5/Cy7 fluorescence ratio every 15 minutes for 2 hours (excitation 630 nm/emission 680-780 nm) using Tecan Infinite M100 pro plate reader (Tecan Laboratories, Switzerland).

### Zymogram

Zymogram was prepared as previously described²². Briefly, 30-40 mg of tissue was homogenized in buffered solution and centrifuged. Tissue samples, along with SeeBlue Plus 2 Protein ladder and MMP standards were loaded on the gel and run at 120V for two hours. Following renaturation, development and staining, gels were imaged and analyzed with Image J. MMP activity of samples was recorded as a percentage of MMP activity within the positive control lane.

### Peptide Synthesis

RACPP and uncleavable-control were synthesized as previously described¹². The RACPP contains a poly-cationic moiety linked to a neutralizing poly-anionic arm via a linker that is cleavable by MMP-2 and MMP-9. A Cy5 fluorophore is attached to the polycationic portion while the Cy7 fluorescent molecule is attached to the poly-anionic domain. Following cleavage by MMPs, the polycationic portion conjugated to Cy5 is dequenched and becomes trapped within nearby tissue. Uncleavable-control peptide lacks an MMP cleavable linker.

### Cell Culture and Mouse Tongue Xenografts

Human tongue squamous cell carcinoma lines SCC-4, SCC-9, SCC-15, and SCC-25 (ATCC) were maintained in Dulbecco's modified Eagle's medium with nutrient mixture F-12 (DMEM/F-12) containing 10% fetal bovine serum (FBS) and supplemented with 400 ng/mL of hydrocortisone. Human tongue squamous cell carcinoma line CAL-27 (ATCC) was maintained in DMEM containing 10% FBS. Cells were incubated at 37° C. in 5% CO₂. Nu/nu mice (age, 3-6 months) were injected with cultured HNSCC cells (˜10⁶ for CAL-27, ˜5×10⁶ for SCC-4, SCC-9, SCC-15, SCC-25) into the tip of the tongue. One cell line was used in each mouse for these experiments (n=22 total; CAL-27:n=5, SCC-25 n=4, SCC-15:n=4, SCC-4:n=4, SCC-9:n=5).

In Vivo Imaging with RACPP

Mice were monitored for 20% weight loss or tumor size >4-5 mm. Once these parameters were met, animals were anesthetized with isoflurane and injected intravenously with RACPP or control uncleavable peptide (0.4 nmol/g). Two hours after injection, mice were re-anesthetized (100 mg/kg ketamine and 5 mg/kg midazolam) and subcutaneous cervical tissue/anterior tongue exposed for imaging (Maestro, CRI). After completion of whole body imaging, animals were euthanized. The entirety of the tongue was immediately extracted and imaged in the dorsal position (Maestro, CRI).

Spectral imaging was carried out by exciting Cy5 at 620 (±10) nm followed by step-wise emission measurements from 640 to 840 nm through a tunable LCD emission filter. For ratio imaging, numerator (Cy5) and denominator (Cy7) images were generated by integrating spectral images over a defined range at 10 nm intervals (660-720 nm for Cy5 and 760-830 nm for Cy7). Ratio images were generated and color-encoded using custom software. The ratio for each pixel was encoded as hue on a blue to red scale and brightness was based on the original Cy5 images. The software also generated monochromatic Cy5/Cy7 images for further processing (see Image analysis and histologic correlation).

### Histology

Immediately following imaging, tongue tissues were embedded in cryopreservative and stored at −80° C. Samples were cryosectioned into 5-μM sections in the same orientation as the whole tongue molecular imaging and stained with hematoxylin and eosin (H&E). The entirety of the tongue was included in the slice, including both tumor and normal tissue. Samples were evaluated by a pathologist blinded to experimental conditions.

### Mapping Histology to Molecular Imaging

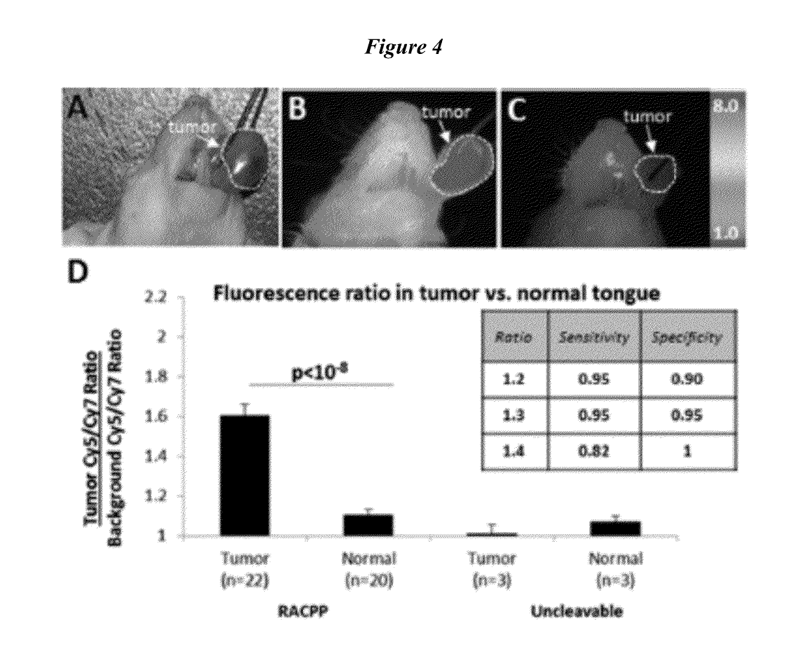

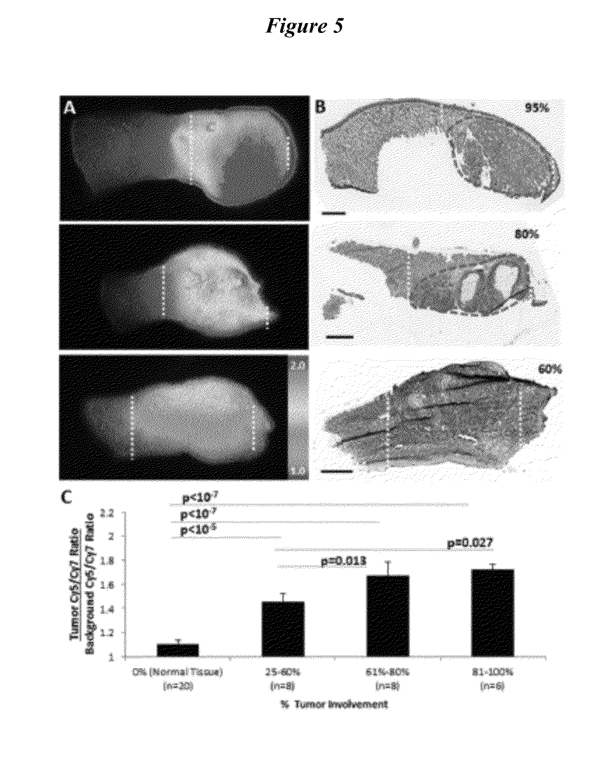

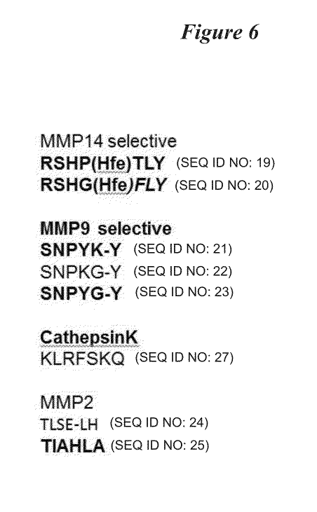

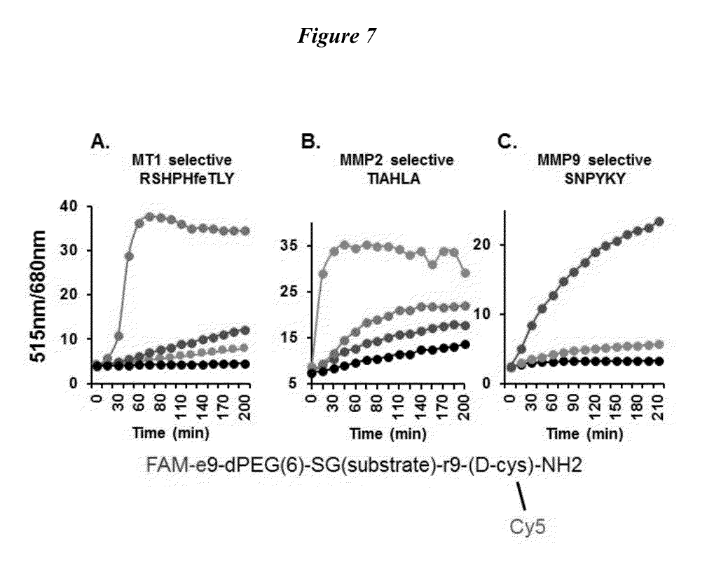

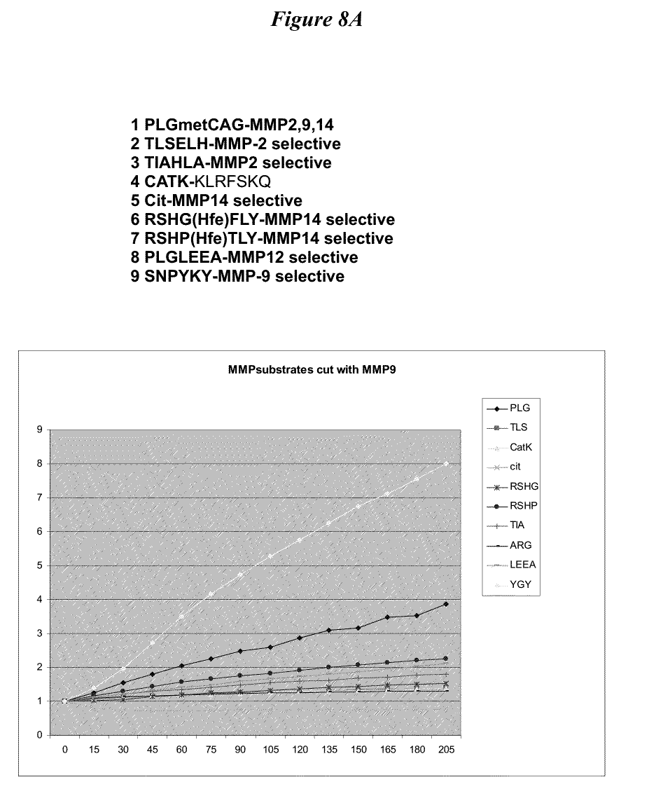

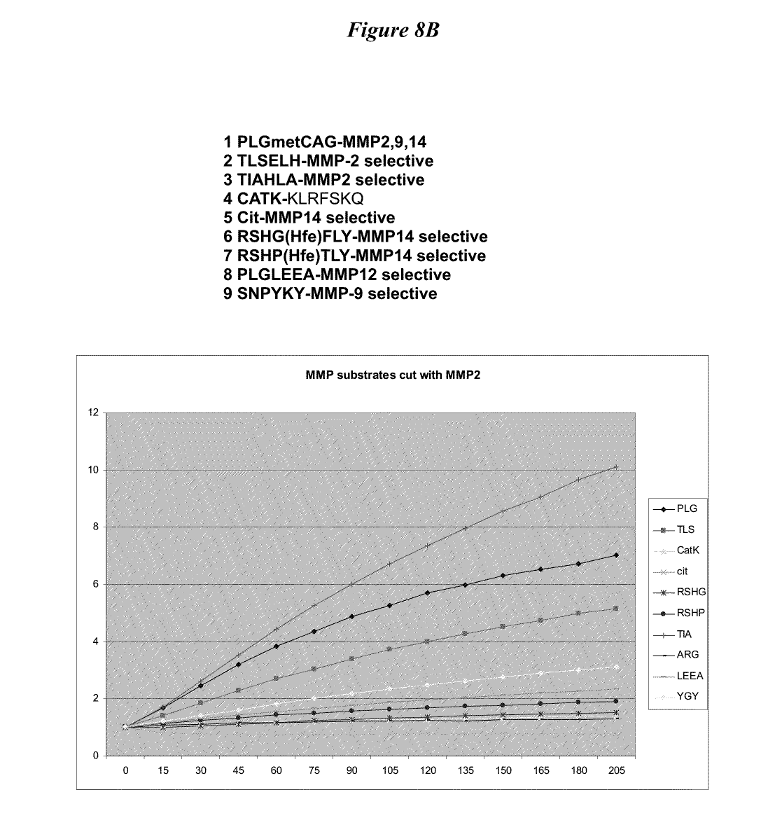

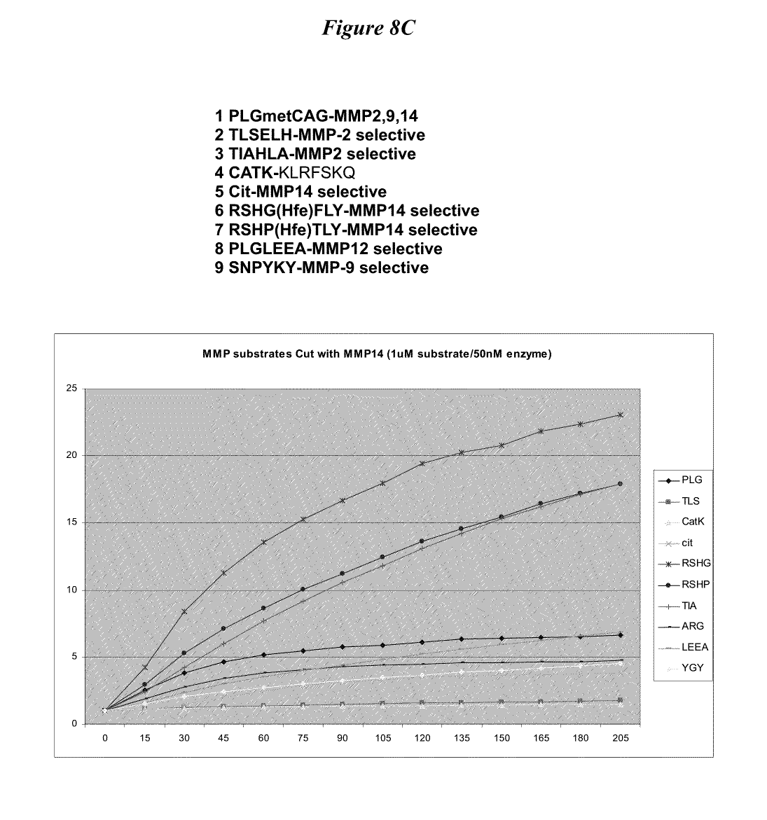

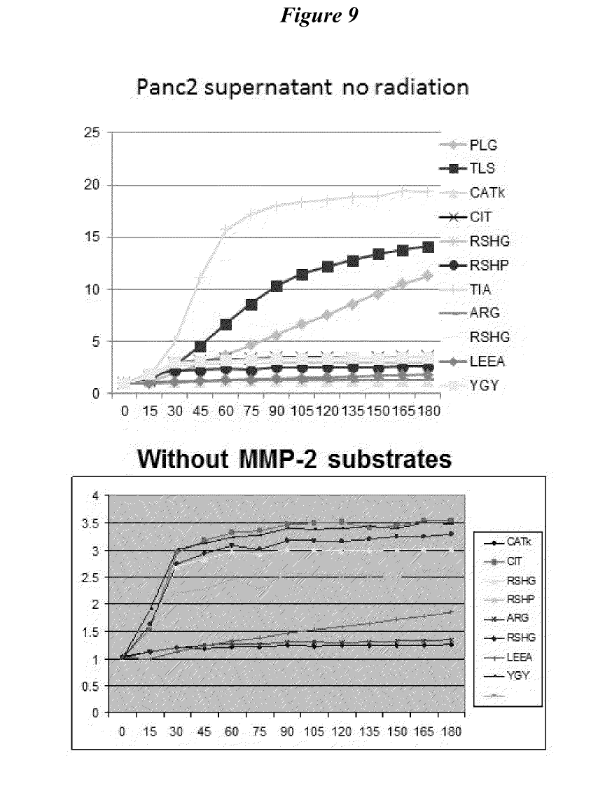

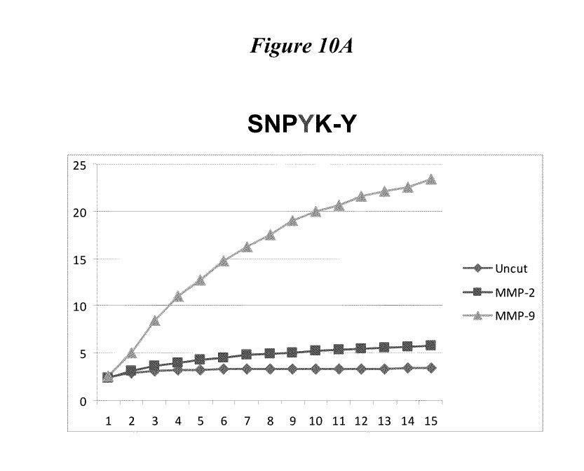

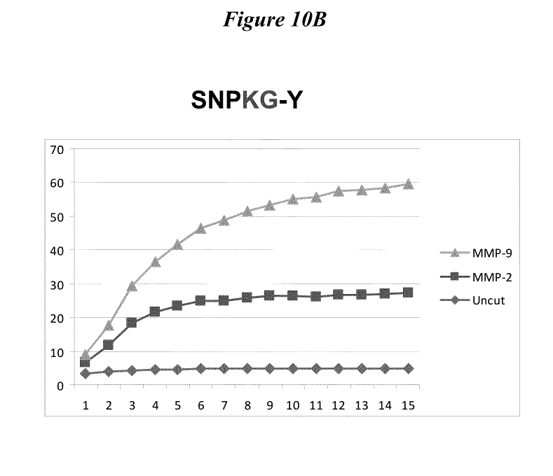

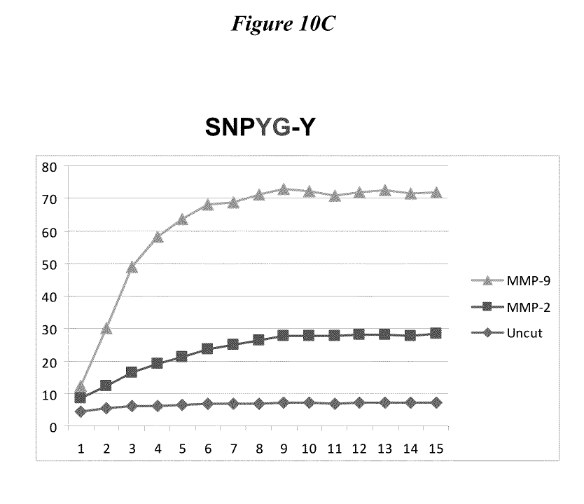

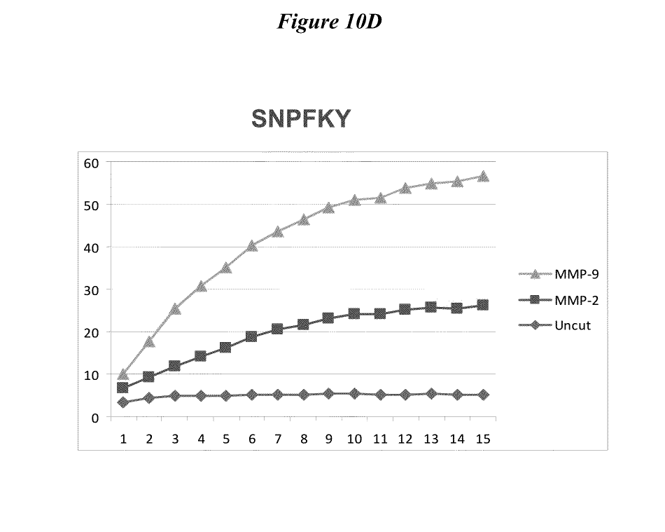

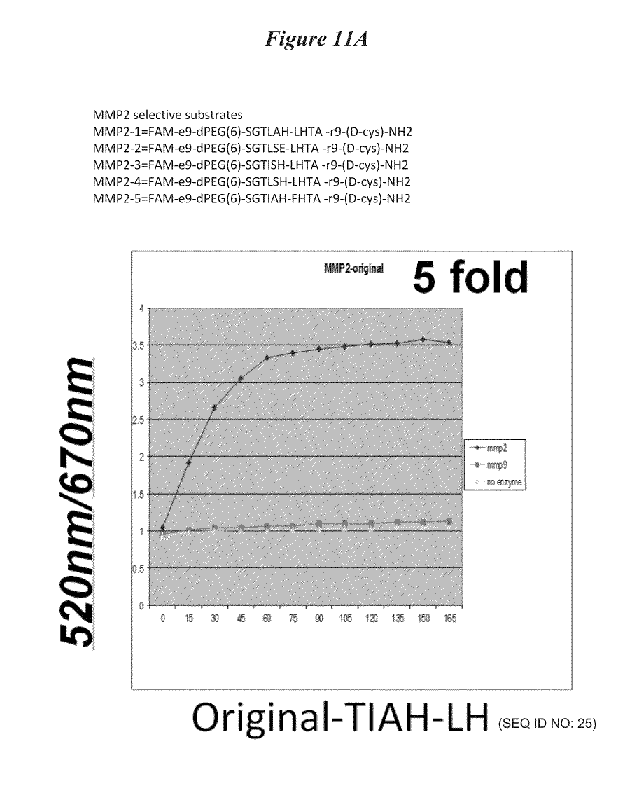

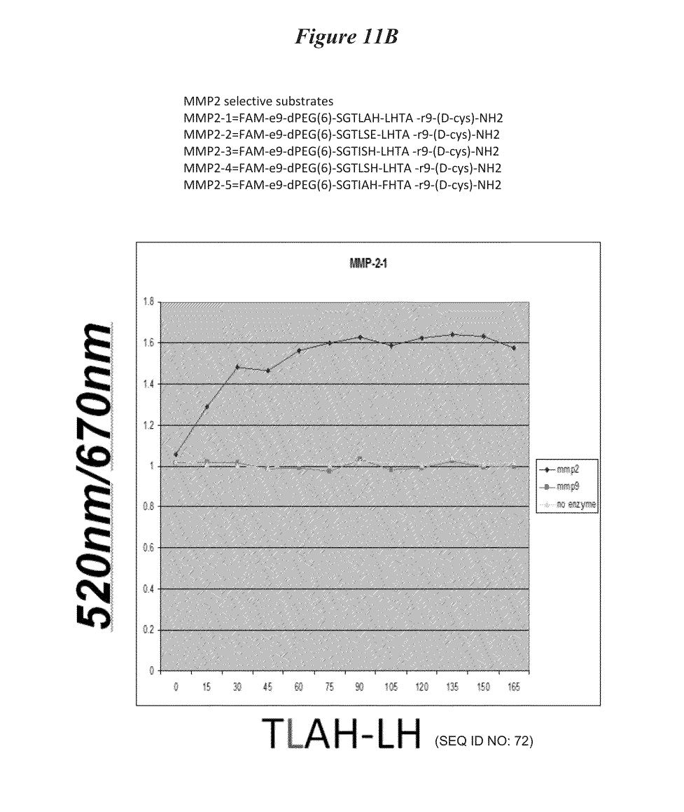

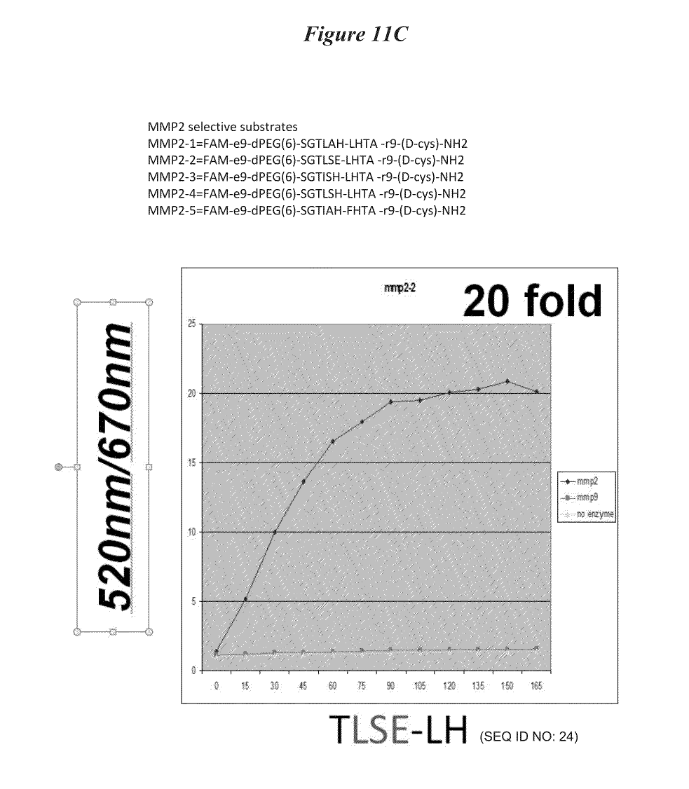

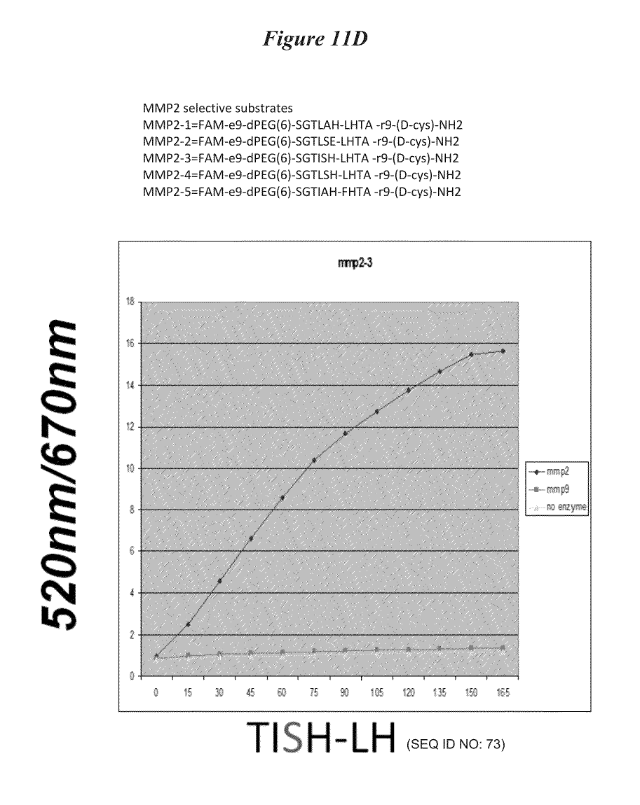

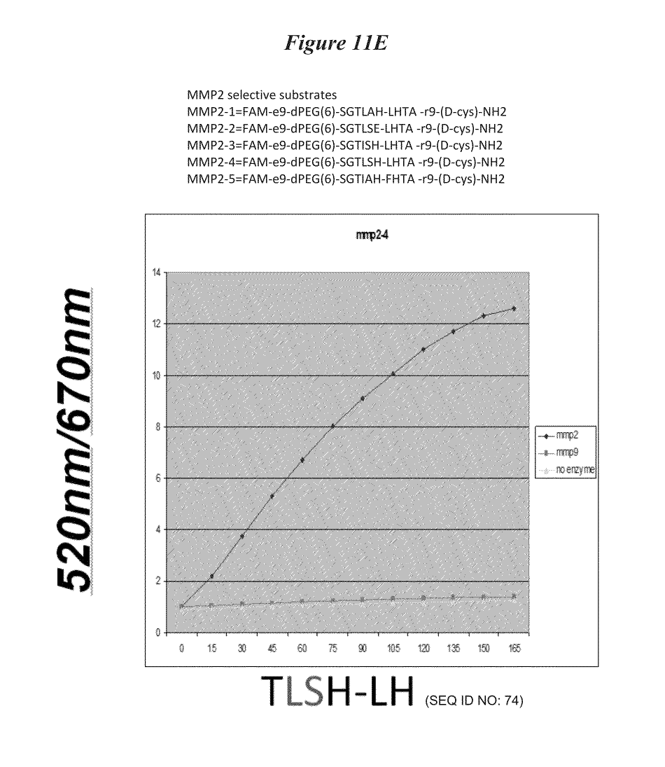

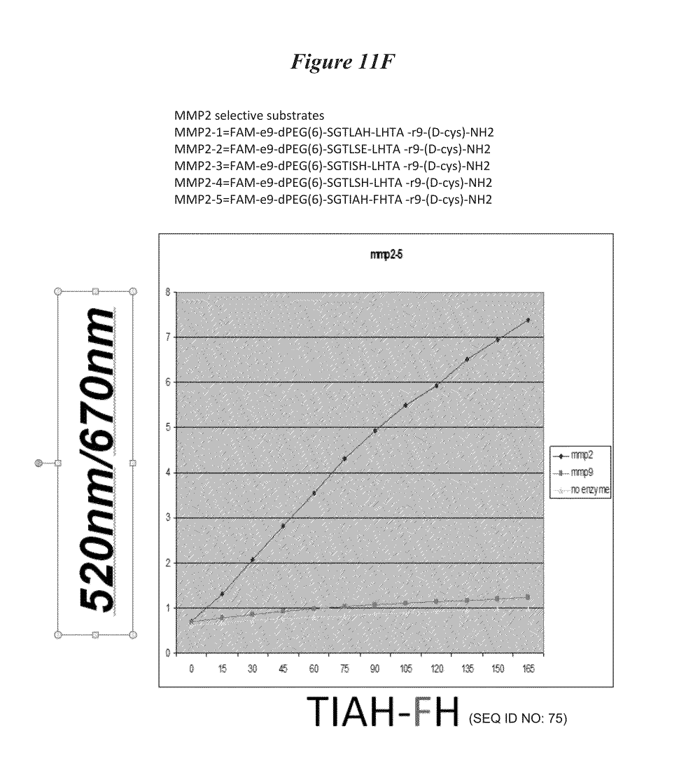

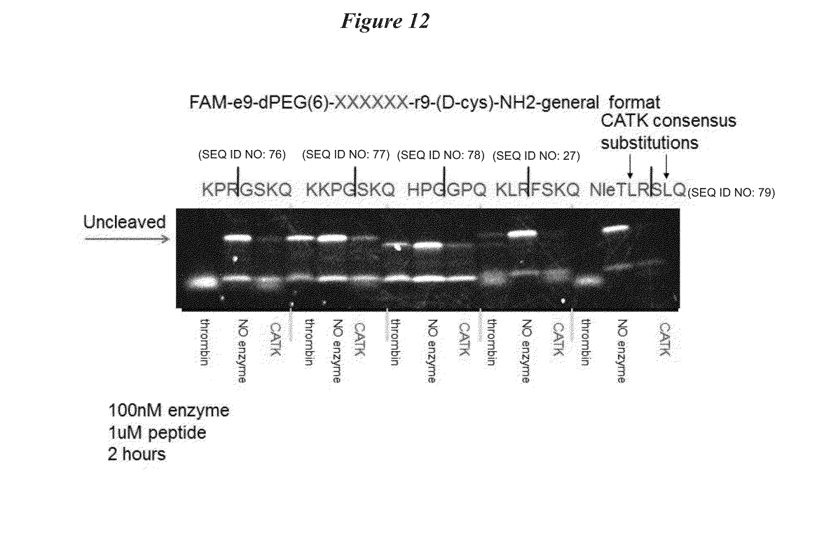

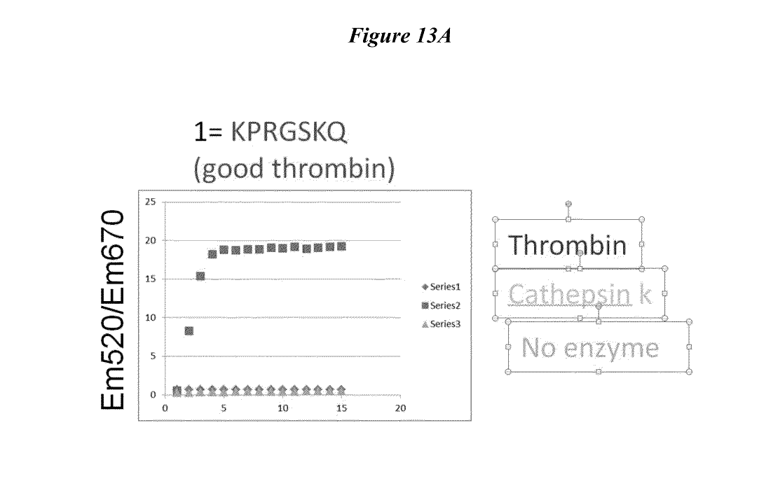

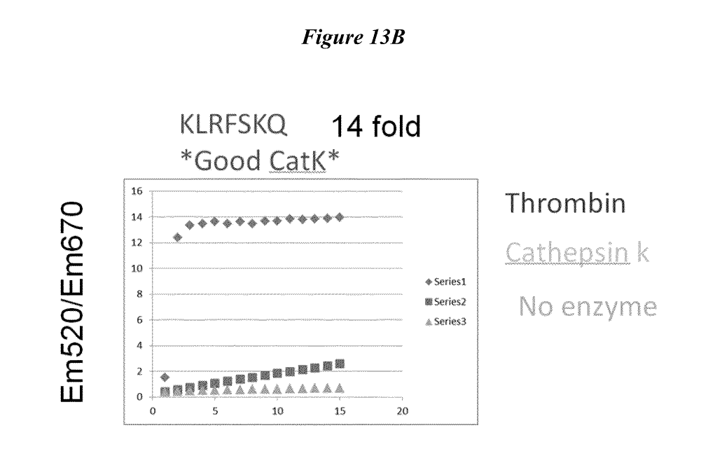

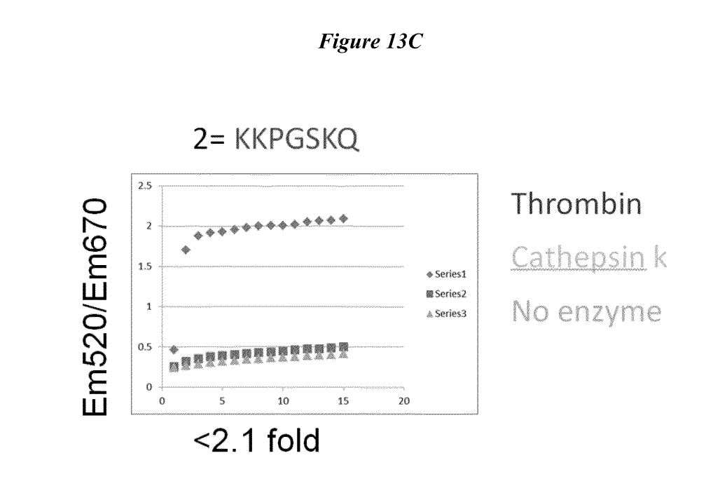

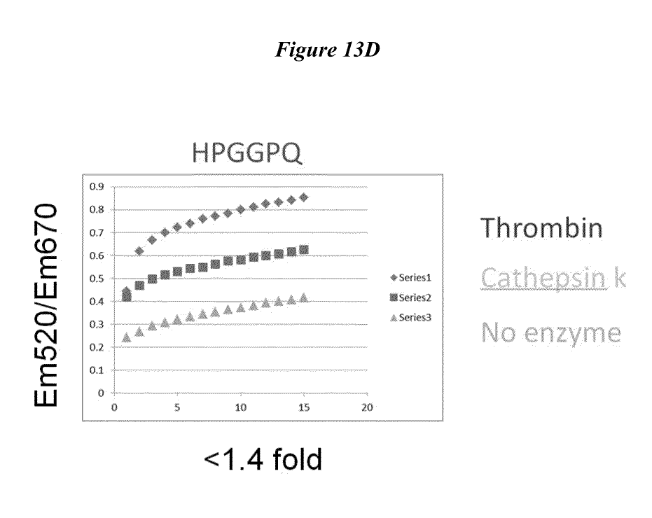

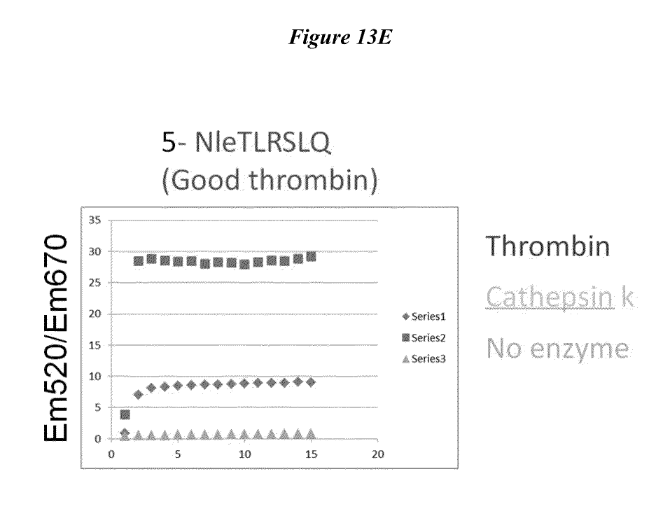

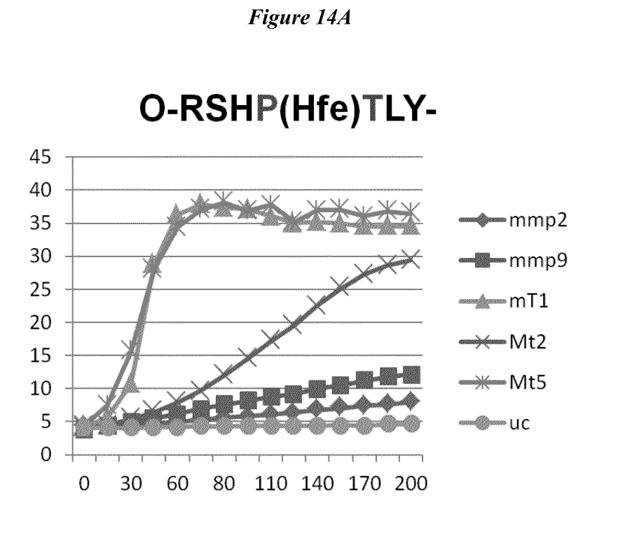

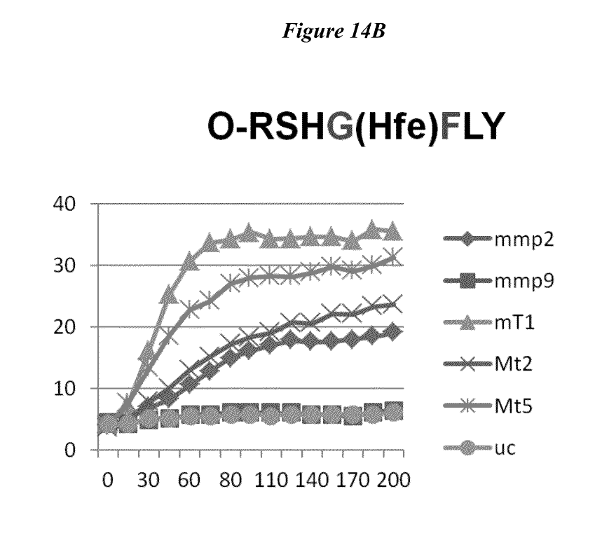

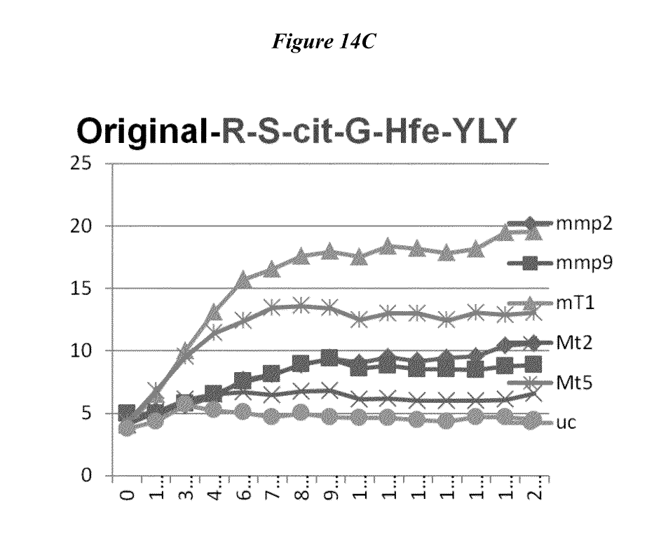

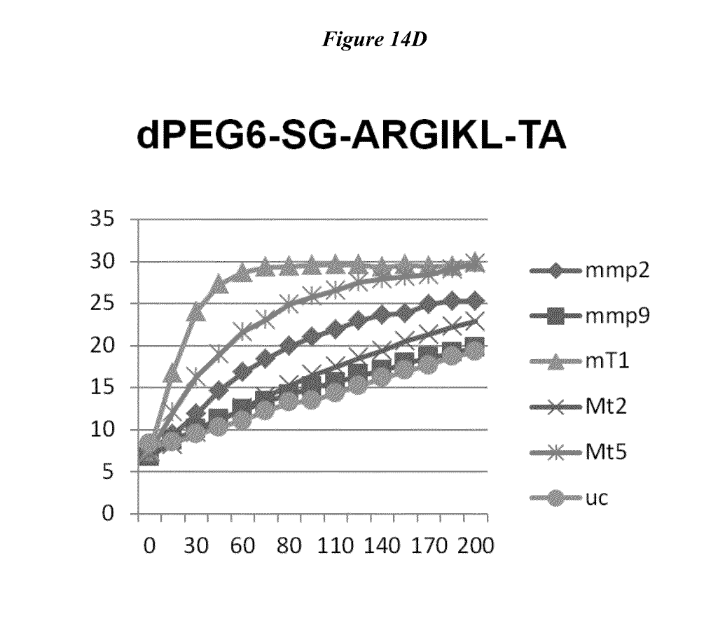

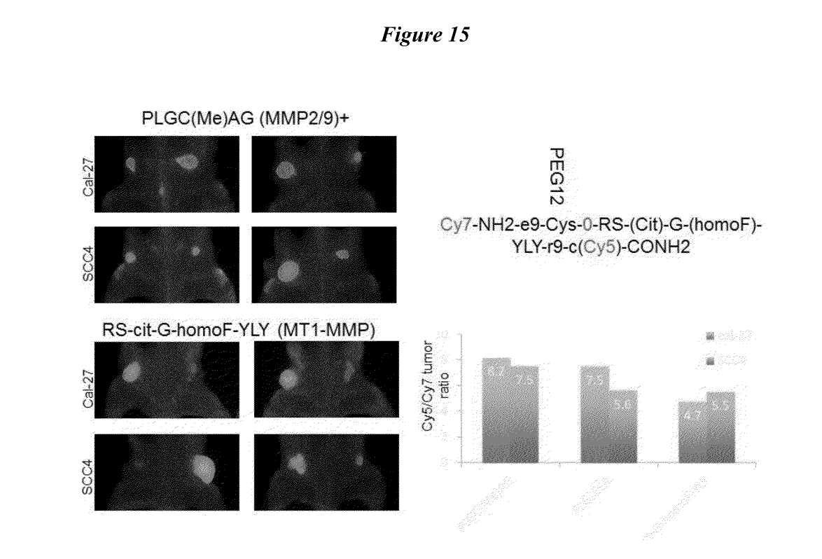

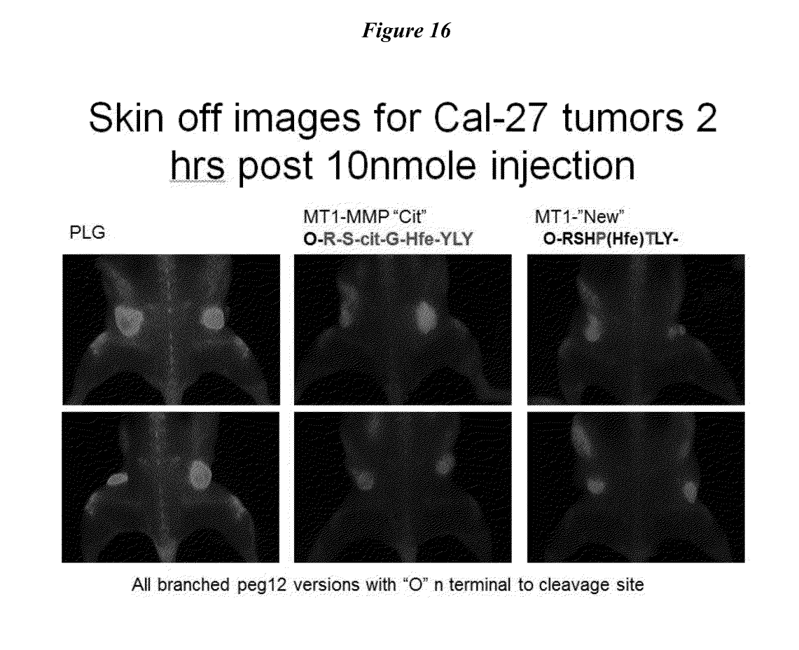

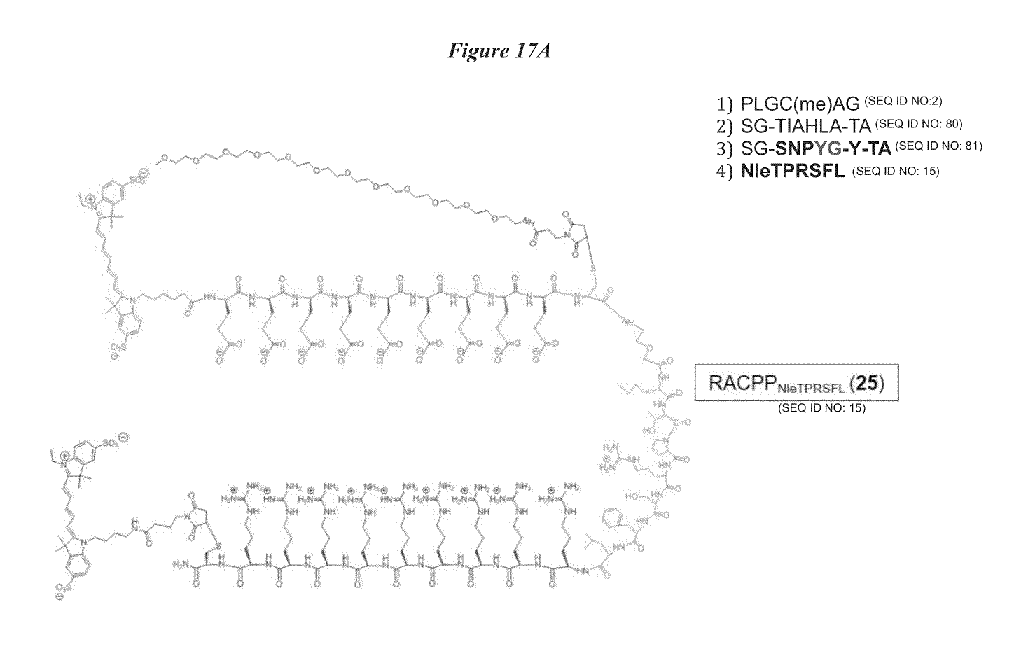

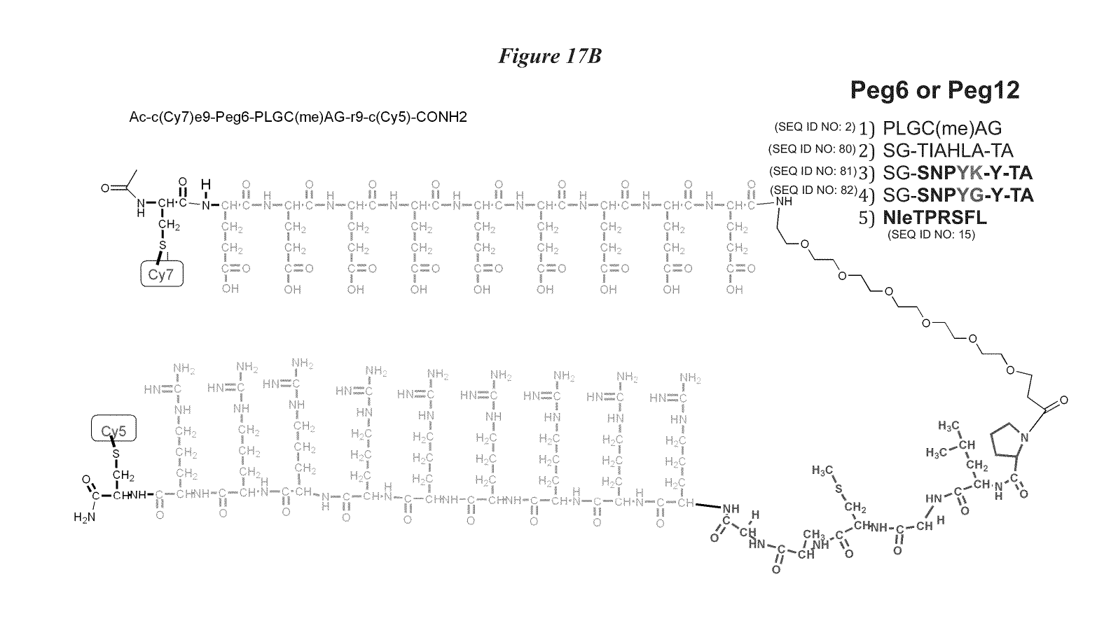

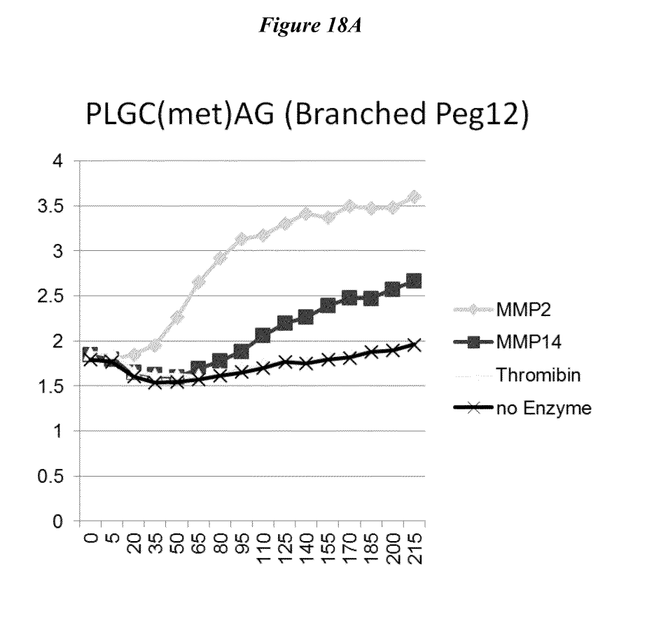

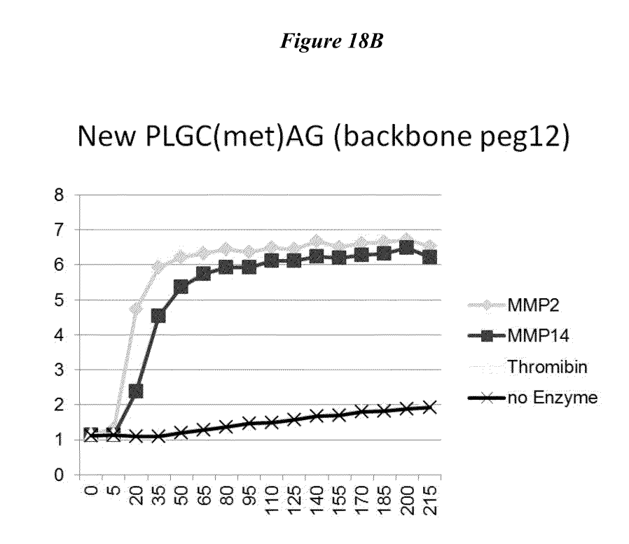

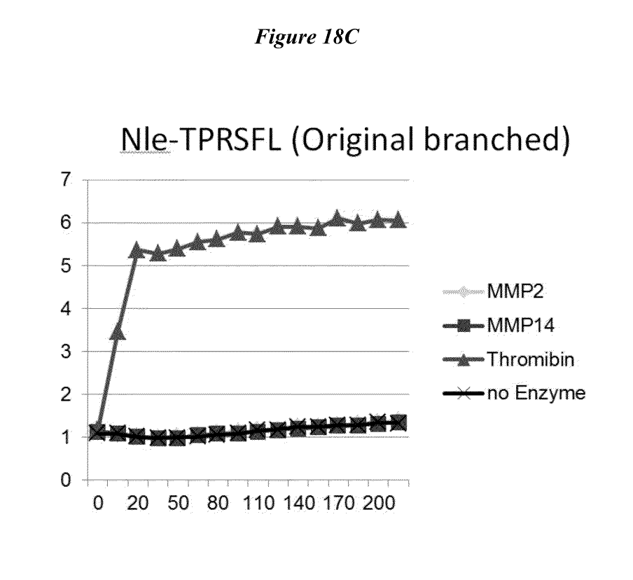

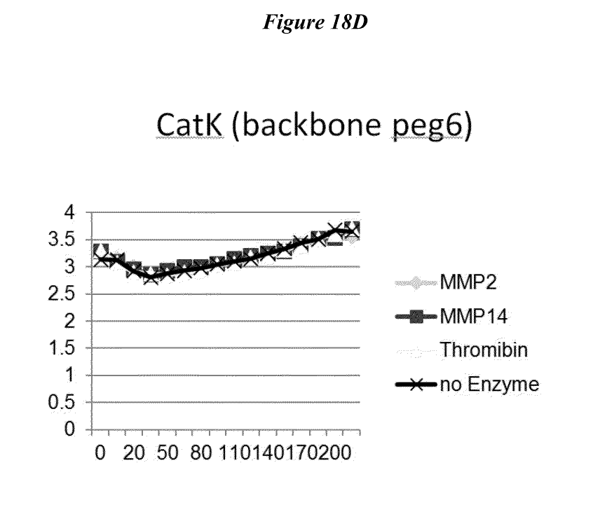

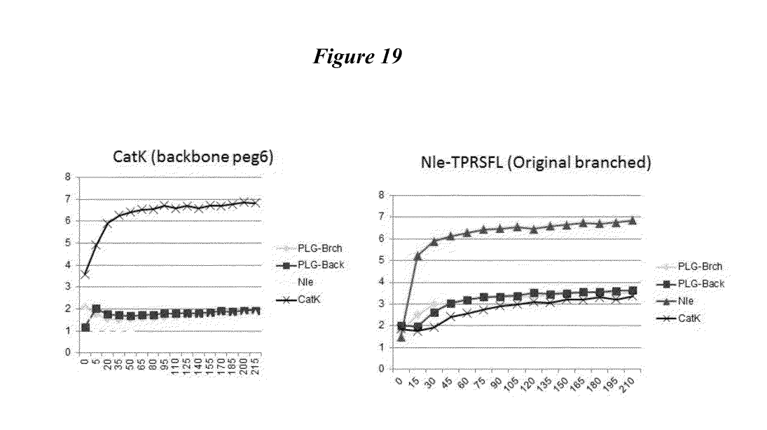

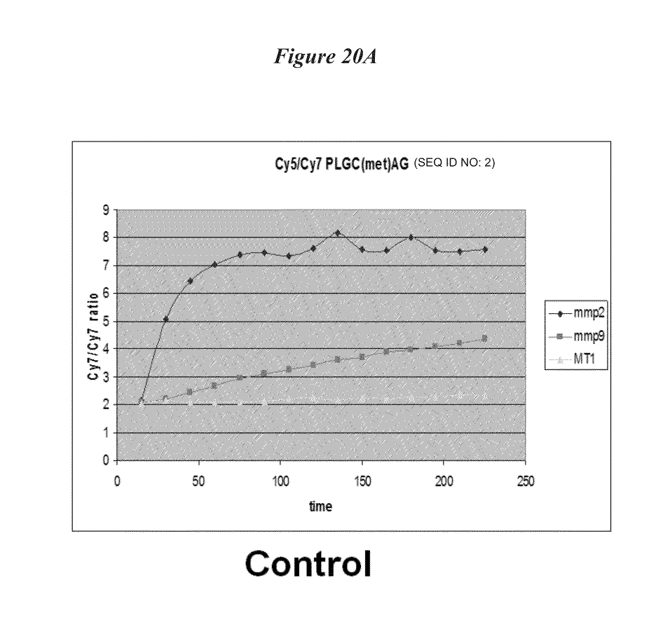

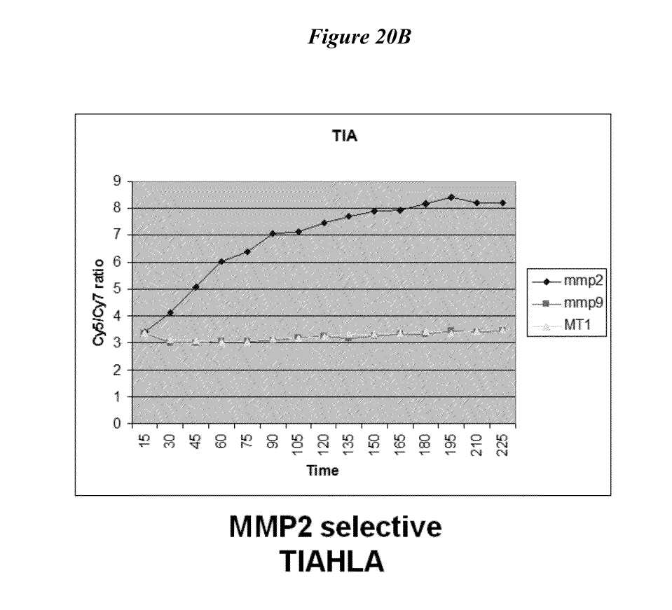

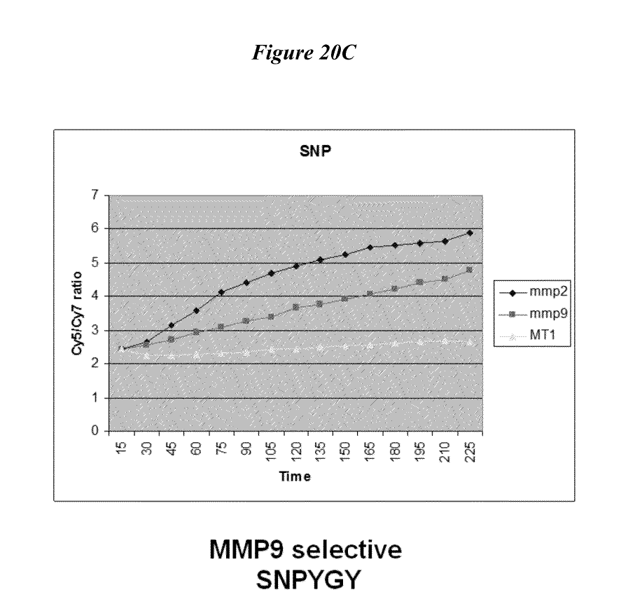

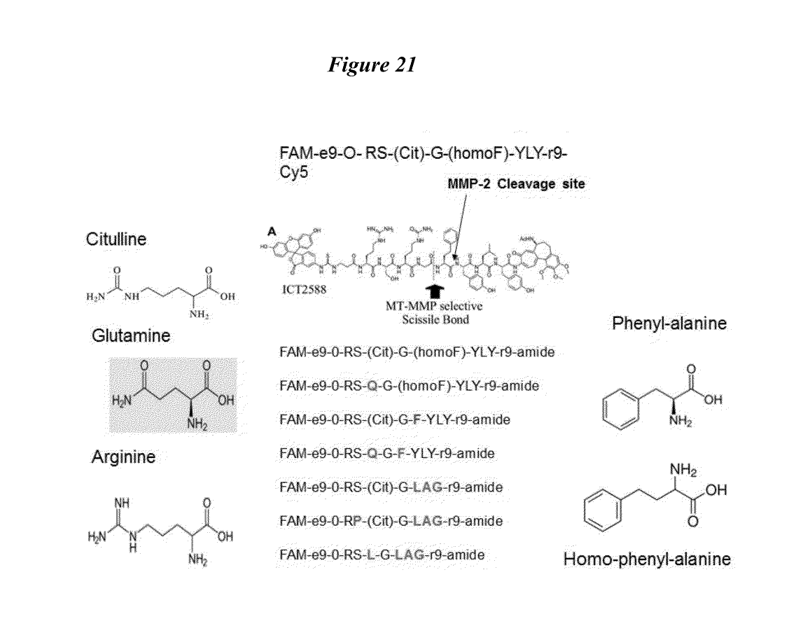

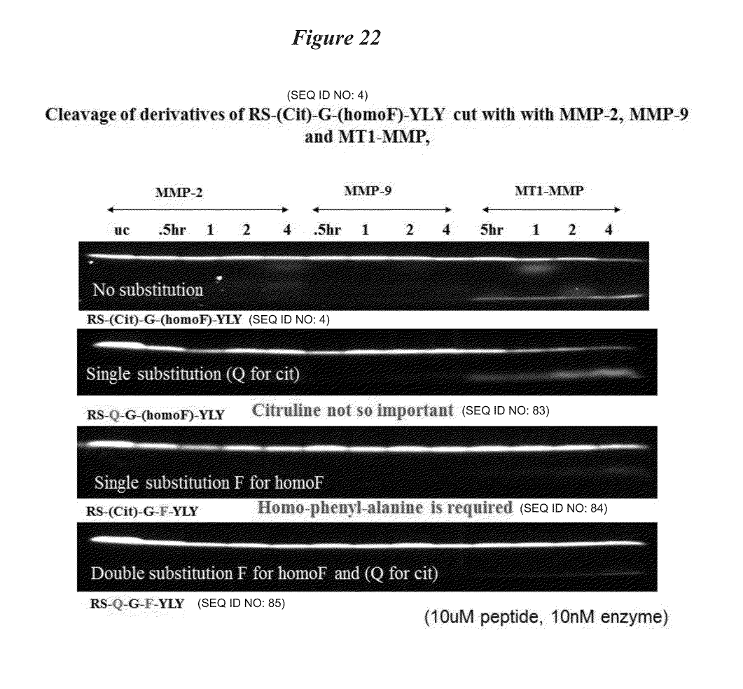

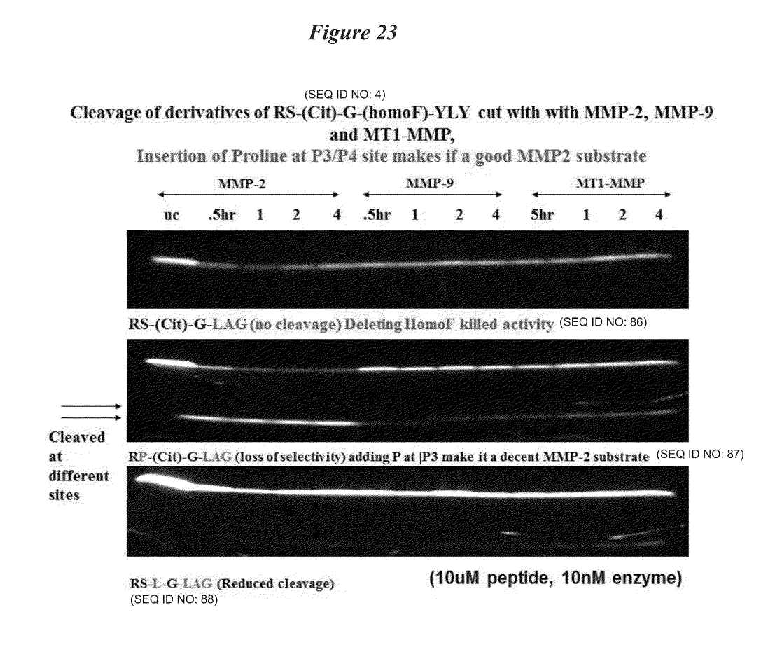

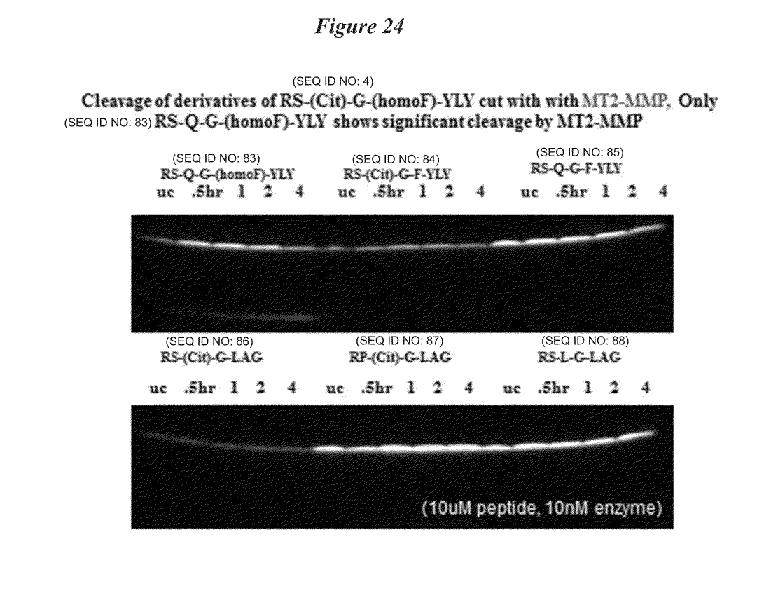

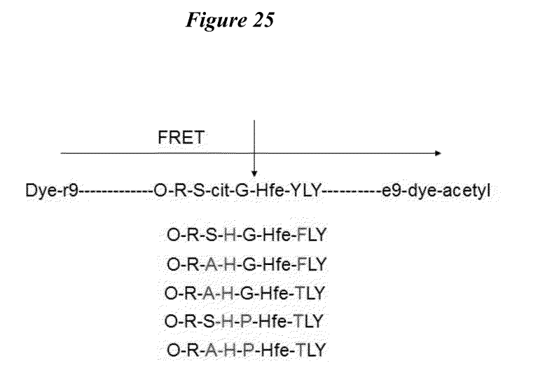

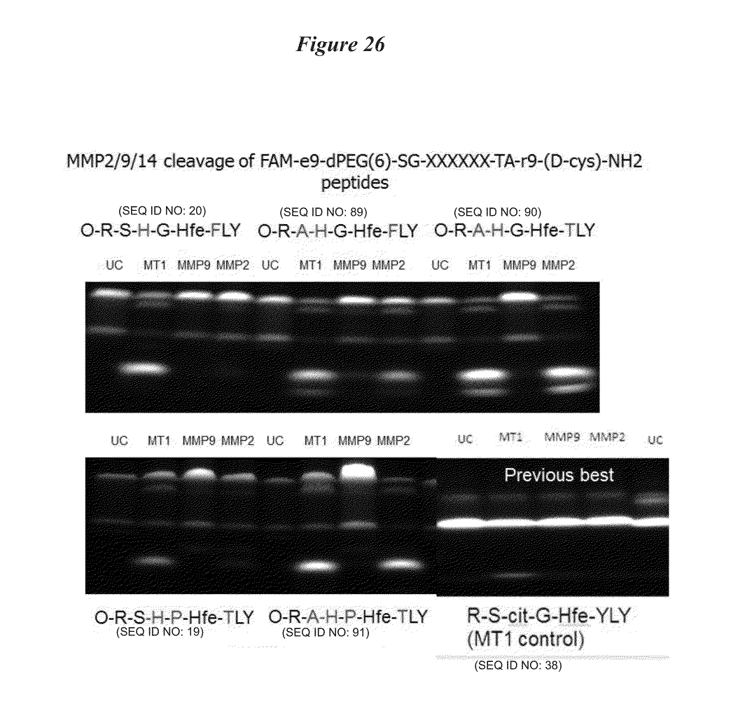

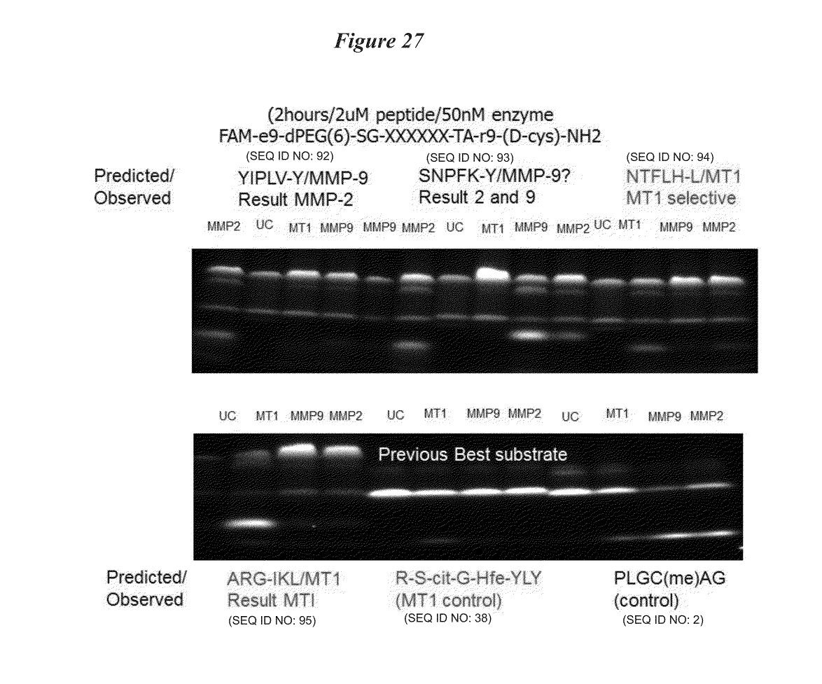

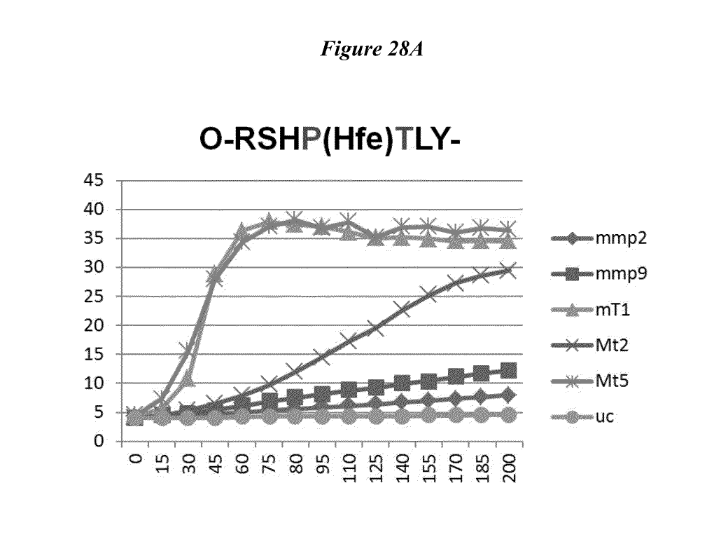

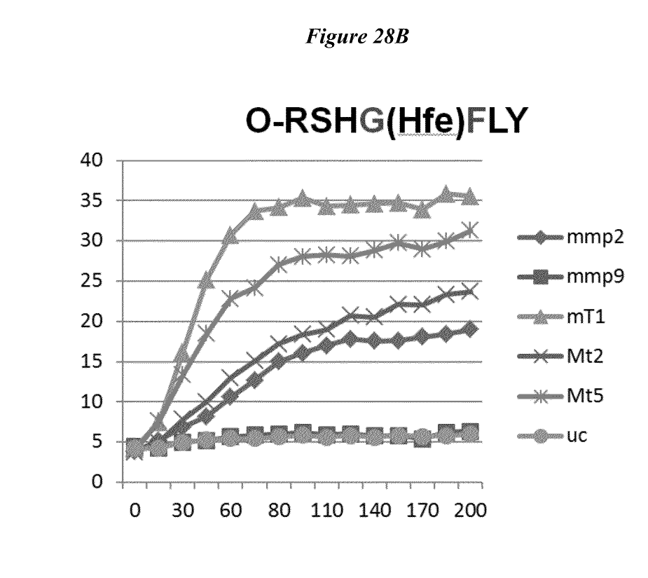

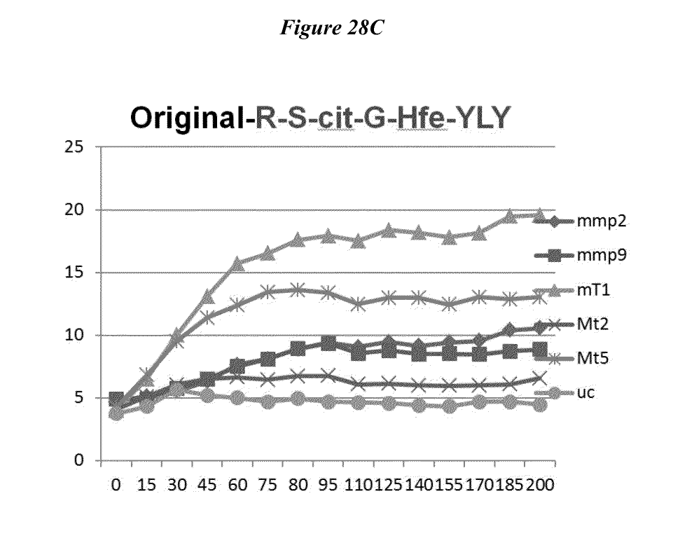

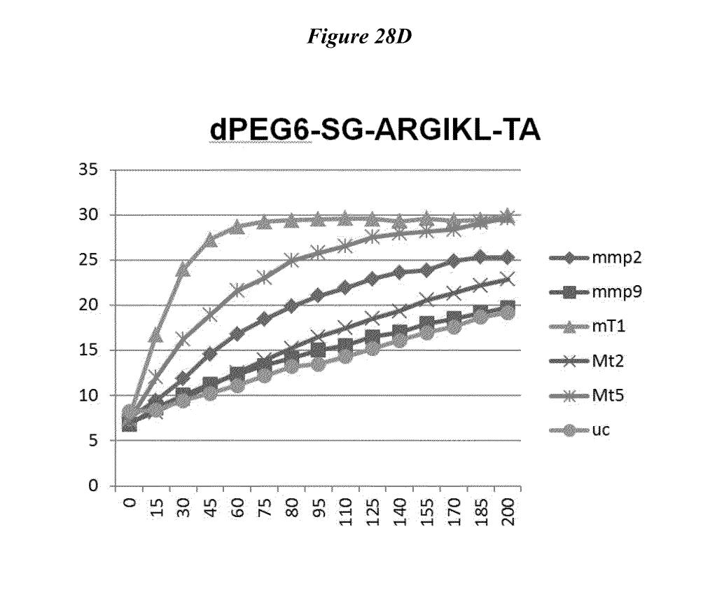

For histologic samples, a pathologist blinded to experimental conditions used a stage micrometer to determine the tumor's linear position and extent along the length of the tongue. This information was mapped to spectral images of the tongue (FIG. 4). A mean Cy5/Cy7 ratio was calculated for segments containing histologically-confirmed tumor and, separately, tumor-free segments.

Percent tumor involvement was approximated by the pathologist as the density of cancerous tissue (vs. non-cancerous tissue) within the tumor-containing segment of tongue (FIG. 4). For example, if the tumor-bearing length of the sample contained only malignant cells and no normal tissue, percent involvement was recorded as 100%. If only half of this region contained malignancy, percent involvement was recorded as 50%. This method for calculating percent tumor involvement has been utilized in other studies^{23,24}.

### Image Processing and Ratio Calculations

Monochromatic Cy5 and Cy7 images were extracted from the spectral image using custom software. Using the “Image Calculator” feature on Image J, the Cy5 image was divided by the Cy7 image to produce a new image, where Cy5/Cy7 ratios were encoded by pixel intensity. Ratios were calculated separately for tumor and normal tongue, which were distinguished based on the histologic map described above. These ratios were each normalized to Cy5/Cy7 ratios of background tissue (cervical soft tissue).

### Statistics

Statistical analysis between experimental groups was conducted using either the 2-tailed independent sample student t test or one-way ANOVA w/post-hoc analysis. Graphical bar-plots were produced using Microsoft Excel, while ROC curves were created with Sigmaplot (12.3). Paired tests were used for TCGA analysis due to matched expression data. For survival analysis, Cox proportional hazards regression was employed using the R ‘survival’ package.

### Results

### MMPs are Overexpressed in HNSCC

To evaluate MMP expression levels in HNSCC from the TCGA, patient-derived tumor specimens were compared with matched normal control tissue. HNSCC tumors showed increased expression of multiple MMPs compared to matched control non-tumor tissue (FIG. 2A, all p values <0.01). Interestingly, MMP14 (also known as MT1-MMP) was the protease with the highest total expression in tumor tissue and had significantly higher expression in tumor compared to matched control tissue (p<10⁻⁵). The second highest expressing MMP in tumor tissue was MMP2. MMP2 and 9 share a common cleavage sequence and they have been particularly well characterized in prior studies in association with HNSCC²⁵. It was found that both MMP2 (p<10⁻¹⁰) and MMP9 (p<10⁻⁶) have significantly greater RNA expression in HNSCC tumors compared to paired-control tissue (n=37, (34 HPV− and 3 HPV+), Wilcoxon signed-rank test).

### MMP 2 and 14 Stratify Survival in HPV+HNSCC

Next, the difference in MMP expression between HPV+ and HPV− tumors was evaluated. It has been found that HPV+ tumors had less overall MMP expression compared to HPV− tumors (FIG. 2B, Kruskal-Wallis test on pooled RNA levels, p<10⁻¹⁰). This is consistent with the hypothesis that HPV+ tumors are less biologically aggressive, and consequently, that these patients tend to have improved survival compared to patients with HPV− tumors. Interestingly, it was found that in patients with HPV+HNSCC, increased expression levels of MMP2 and MMP14 correlated with worse survival (FIG. 2C, 2D p<0.01). Patients with HPV+ tumors who have the highest MMP2 and MMP14 expression (FIG. 2C, 2D red lines) had significantly worse 5 year survival compared to patients with the lowest expression levels of these proteases (FIG. 2C, 2D blue lines). Additionally, for a given patient with HPV+ tumor, there is a significant correlation between MMP2 and MMP14 expression (Spearman Rho=0.56, p<10⁻⁴). Thus, poor prognosis HPV+ tumors stratified in the highest quartiles of MMP2 expression are also likely to have higher expression of MMP14. The same correlation in MMP expression with survival in patients with HPV− tumors was not found. The cause of this is multifactorial and likely related to the observation that HPV− tumors have more genetic mutations compared HPV+ tumors²⁶.

### Zymography

To confirm MMP2/9 activity in mouse HNSCC xenografts, cleavage of gelatin by tumor homogenates via zymography was measured. A two-fold increase in MMP9 and a 13-fold increase in MMP2 activity in HNSCC xenografts compared to normal mouse tongue tissue was found.

### RACPP in Ex-Vivo HNSCC

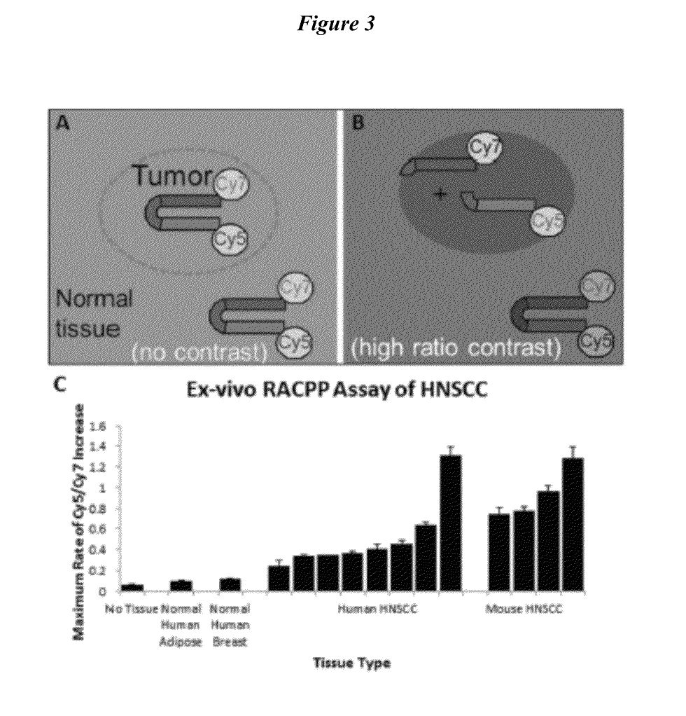

To evaluate ex-vivo MMP2/9 activity in human and mouse HNSCC specimens, the maximum rate of Cy5/Cy7 ratio change over time in homogenates following addition of RACPP (FIG. 3A, 3B) was measured. It was found that patient derived HNSCC specimens show higher MMP2/9 activity compared to non-tumor tissue (FIG. 3C) (ROC Analysis: AUC=1.000, p=0.01). Similarly, mouse HNSCC xenografts also show higher Cy5/Cy7 rate change, signifying higher MMP2/9 activity compared to non-tumor tissue (ROC Analysis: AUC=1.000, p=0.03).

### RACPP Improves Detection of HNSCC

To test tumor-dependent Cy5/Cy7 ratiometric change in living mice, tongue tumor bearing nu/nu mice were intravenously injected with RACPP (n=25). Multispectral imaging of these live, anesthetized mice (ex 620, em 640-840 nm, Maestro, CRI at 2 hours after injection) with both tongue and subcutaneous cervical tissue exposed was conducted(FIG. 4A). The tongue was excised and ultispectral imaging of the tongue performed. Histologic information regarding tumor location and size was correlated and mapped to ratiometric fluorescence image of the tongue. Sample (tumor and non tumor tissue) Cy5/Cy7 ratios were divided by “background” subcutaneous cervical tissue Cy5/Cy7 ratio to compute a “normalized Cy5/Cy7 ratio”¹².

It was found that higher Cy5/Cy7 ratiometric fluorescence in tumor (FIG. 4B, red color) compared to adjacent normal tongue (FIG. 4B, tan color). Injections of our control (uncleavable) probe revealed no ratiometric difference between tongue tumor and normal tongue (FIG. 4C). Following intravenous administration of MMP2/9-cleavable RACPP, mice showed greater normalized Cy5/Cy7 ratio in tumor (1.61±0.05, n=22) compared to normal tongue (1.11±0.03, n=20, p<10⁻⁸). This increase in ratiometric fluorescence in orthotopic tumors was not seen following intravenous injection of uncleavable control probe (tumor=1.01±0.04, n=3; normal tongue=1.07±0.03, n=3, p=0.30) (FIG. 4D).

The receiver-operating curve (ROC) for cleavable RACPP revealed an area under the curve (AUC) of 0.995±0.006 (p<10⁻⁴) with a peak sensitivity of 95% and peak specificity of 100% for a normalized ratio cutoff of 1.345 (FIG. 4D insert). The two tumor specimens not detected by this threshold-cutoff had relatively low tumor burden (<60% involvement, see below).

### RACPPs Enable Stratification of Tumor Burden

One critical component of intraoperative margin evaluation is determining how much tumor burden is present at the edges of the surgical field. To evaluate the ability of RACPP to stratify tissue with variable tumor burden, percent involvement of cancer within tumor-bearing portions of each sample was approximated by a pathologist blinded to experimental conditions. It was found that varying levels of tumor burden among the 22 samples ranging from 25-100% invasion (FIG. 5A). To evaluate the stratification of ratiometric fluorescence values between different levels of tumor burden, samples were statistically separated into the following tertiles of cancer involvement: 25-60% (n=8), 61-80% (n=8), 81-100% (n=6) (FIG. 5B). Adjusted Cy5/Cy7 ratios were computed for each tertile and compared with normal tongue tissue (n=20).

It was found that all tertiles of varying tumor burden showed significantly greater normalized Cy5/Cy7 ratio than normal tongue tissue (FIG. 5C; lowest tertile of tumor involvement=1.46±0.07, p<10⁻⁵, middle tertile=1.67±0.12, p<10⁻⁷, highest tertile=1.72±0.04, p<10⁻⁷). Additionally, tumors with percent involvement in the highest and middle tertiles showed significantly greater normalized ratios than the lowest tertile (p=0.01 for highest vs. lowest tertile, p=0.03 for middle vs. lowest tertile). Future experiments will focus on evaluating the ability of RACPP to detect incrementally smaller levels of tumor burden (i.e. from 1% to 25% involvement).

### DISCUSSION

In this study, mRNA expression levels for MMPs in human HNSCC were analyzed using The Cancer Genomic Atlas (TCGA), the largest available collection of human HNSCC specimens. The prognostic value of MMP overexpression in terms of survival in patients with HPV+ and HPV−HNSCC tumors was evaluated. It was found that many MMPs are overexpressed in HNSCC tumors compared to paired control tissue. However, patients with HPV+HNSCC tumors have significantly lower overall MMP levels compared to patients with HPV−HNSCC tumors. This finding is consistent with previous studies showing that patients with HPV+HNSCC tumors have better overall survival compared to patients with HPV−HNSCC tumors²⁷.

Of the various MMPs thought to be involved in cancer, attention has focused on MMP2/9 because they are overexpressed in a variety of malignant tumors and their expression is often associated with tumor grade and poor patient prognosis. Interestingly, it was found that of the MMPs that are increased in tumor compared to control tissue, MMP2 and MMP14 are expressed at higher levels compared to all other MMPs, suggesting that these proteinases may be particularly important in HNSCC. Furthermore, it was found that in patients with HPV+HNSCC tumors, increased MMP2 and MMP14 expression levels correlated with worse overall survival. If clinically validated in a prospective trial, the increases in MMP2 and MMP14 represent two molecular biomarkers that can individualize management of patients with HPV+ tumors.

Using MMP2/9 cleavable RACPP, it was found that ex-vivo human HNSCC specimens show greater activity compared to normal tissue. This finding correlates with previous studies demonstrating higher MMP2/9 expression at the invasive edge of tumors²⁸. The high sensitivity and specificity of RACPPs to differentiate between tumor and normal tissue suggests that ex-vivo measurements of MMP2/9 activity in HNSCC specimens may complement MMP mRNA expression studies in evaluating patient prognosis and in determining which patients would benefit from RACPP guided surgery.

In multiple human cell line models of HNSCC xenografts, it was found that higher MMP2/9 activity as evidenced by gelatinase zymography and higher ratiometric fluorescence signal following systemically applied RACPP compared to non-tumor tissue. All ratios were computed from histologically confirmed tumor or normal tissue, eliminating verification bias. The ideal discrimination threshold for detecting cancer versus normal tissue is 1.345, which is consistent with previously reported ratiometric thresholds for this probe¹². Our study tested multiple tongue squamous cell carcinoma cell lines from ATCC to highlight the RACPP's broad applicability.

One critical component of intraoperative margin evaluation is determining how much tumor burden is present at the edges of the surgical field. It was found that within tumor bearing tissue, the greater the tumor burden, the greater the ratiometric fluorescence signal following intravenous RACPP administration. Percent tumor involvement has been shown to be important for survival and recurrence outcomes in prostate and breast cancer^{23,29}. The correlation between intraoperative ratiometric fluorescence level and tumor burden suggests that RACPP can improve intraoperative decision making by providing information regarding local level of tumor involvement and consequently margin clearance.

### REFERENCES FOR EXAMPLE 4

1. Ferlay J, Shin H R, Bray F, Forman D, Mathers C, Parkin D M. Estimates of worldwide burden of cancer in 2008: GLOBOCAN 2008. Int J Cancer 2010; 127:2893-2917.
2. D'Souza G, Kreimer A R, Viscidi R et al. Case-control study of human papillomavirus and oropharyngeal cancer. N Engl J Med 2007; 356:1944-1956.
3. Shin D M, Khuri F R. Advances in the management of recurrent or metastatic squamous cell carcinoma of the head and neck. Head Neck 2013; 35:443-453.
4. Uloza V, Liutkevicius V, Pangonyte D, Saferis V, Lesauskaite V. Expression of matrix metalloproteinases (MMP-2 and MMP-9) in recurrent respiratory papillomas and laryngeal carcinoma: clinical and morphological parallels. Eur Arch Otorhinolaryngol 2011; 268:871-878.
5. Wittekindt C, Jovanovic N, Guntinas-Lichius O. Expression of matrix metalloproteinase-9 (MMP-9) and blood vessel density in laryngeal squamous cell carcinomas. Acta Otolaryngol 2011; 131:101-106.
6. Liu W W, Zeng Z Y, Wu Q L, Hou J H, Chen Y Y. Overexpression of MMP-2 in laryngeal squamous cell carcinoma: a potential indicator for poor prognosis. Otolaryngol Head Neck Surg 2005; 132:395-400.
7. Mallis A, Teymoortash A, Mastronikolis N S, Werner J A, Papadas T A. MMP-2 expression in 102 patients with glottic laryngeal cancer. Eur Arch Otorhinolaryngol 2012; 269:639-642.
8. Zhou C X, Gao Y, Johnson N W, Gao J. Immunoexpression of matrix metalloproteinase-2 and matrix metalloproteinase-9 in the metastasis of squamous cell carcinoma of the human tongue. Aust Dent J 2010; 55:385-389.
9. Chaudhary A K, Singh M, Bharti A C, et al. Genetic polymorphisms of matrix metalloproteinases and their inhibitors in potentially malignant and malignant lesions of the head and neck. J Biomed Sci 2010; 17:10.
10. Olson E S, Aguilera T A, Jiang T et al. In vivo characterization of activatable cell penetrating peptides for targeting protease activity in cancer. Integr Biol (Camb) 2009; 1:382-393.
11. Aguilera T A, Olson E S, Timmers M M, Jiang T, Tsien R Y. Systemic in vivo distribution of activatable cell penetrating peptides is superior to cell penetrating peptides. Integr Biol 2009; 1:371-381.
12. Savariar E N, Felsen C N, Nashi N et al. Real-time in vivo molecular detection of primary tumors and metastases with ratiometric activatable cell-penetrating peptides. Cancer Res 2013; 73:855-864.
13. Savariar E N, Felsen C N, Nashi N et al. Real-time in vivo molecular detection of primary tumors and metastases with ratiometric activatable cell-penetrating peptides. Cancer Res 2013; 73:855-864.
14. Hague R, Contreras R, McNicoll M P, Eckberg E C, Petitti D B. Surgical margins and survival after head and neck cancer surger. BMC Ear Nose Throat Disord 2006; 16:2.
15. Singletary S. Surgical margins in patients with early-stage breast cancer treated with breast conservation therapy. Am J Surg 2002; 184:383-393.
16. Nagtegaal I D, Quirke P. What is the role for the circumferential margin in the modern treatment of rectal cancer? J Clin On 2008; 26:303-312.
17. Meric F, Mirza N, Vlastos G et al. Positive surgical margins and ipsilateral breast tumor recurrence predict disease-specific survival after breast-conserving therapy. Cancer 2003; 97:926-933.
18. Snijder R, de la Riviere A, Elbers H, van den Bosch J. Survival in resected stage I lung cancer with residual tumor at the bronchial resection margin. Annals of Thoracic Surg 1998; 65.
19. Dotan Z, Kavanagh K, Yossepowitch 0 et al. Positive surgical margins in soft tissue following radical cystectomy for bladder cancer and cancer specific survival. J Urol 2007; 178:2308-2312.
20. Wieder J A, Soloway M S. Incidence, etiology, location, prevention and treatment of positive surgical margins after radical prostatectomy for prostate cancer. J Urol 1998; 160:299-315.
21. Gross A, Orosco R, Shen J et al. A prognostic model of head and neck cancer ties TP53 mutation to 3p loss. Nature Genetics [Under Review].
22. Toth M, Sohail A, Fridman R. Assessment of gelatinases (MMP-2 and MMP-9) by gelatin zymography. Methods Mol Biol 2012; 878:121-35.
23. Rampersaud E N, Sun L, Moul J W, Madden J, Freedland S J. Percent tumor involvement and risk of biochemical progression after radical prostatectomy. J Urol 2008; 180:571-576; discussion 576.
24. Thompson I M, 3rd, Salem S, Chang S S et al. Tumor volume as a predictor of adverse pathologic features and biochemical recurrence (BCR) in radical prostatectomy specimens: a tale of two methods. World J Urol 2011; 29:15-20.
25. Xia T, Akers K, Eisen A Z, Seltzer J L. Comparison of cleavage site specificity of gelatinases A and B using collagenous peptides. Biochim Biophys Acta 1996; 1293(2):259-266.
26. Maruyama H, Yasui T, Ishikawa-Fujiwara T, et al. Human papillomavirus and p53 mutations in head and neck squamous cell carcinoma among Japanese population. Cancer Sci 2014. [In Press]
27. Sivars L, Nasman A, Tertipis N et al. Human papillomavirus and p53 expression in cancer of unknown primary in the head and neck region in relation to clinical outcome. Cancer Med 2014; 3(2): 376-84.
28. Kuniyasu H, Troncoso P, Johnston D et al. Relative expression of type IV collagenase, E-cadherin, and vascular endothelial growth factor/vascular permeability factor in prostatectomy specimens distinguishes organ-confined from pathologically advanced prostate cancers. Clin Cancer Res 2000; 6(6): 2295-308.
29. Kohrt H E, Nouri N, Nowels K, Johnson D, Holmes S, Lee P P. Profile of immune cells in axillary lymph nodes predicts disease-free survival in breast cancer. PLoS Med 2005; 2:e284.

### Example 5

### Matrix-Metalloproteinases in Head and Neck Carcinoma-Cancer Genome Atlas Analysis and Fluorescence Imaging in Mice

### Abstract:

### Objective:

Obtain matrix-metalloproteinase(MMP) expression profiles for head and neck squamous cell carcinoma(HNSCC) specimens from The Cancer Genomic Atlas (TCGA)

Demonstrate HNSCC imaging using MMP-cleavable, fluorescently-labeled ratiometric activatable cell-penetrating peptide(RACPP).

Study Design:

Retrospective human cohort study; prospective animal study

Setting:

Translational Research Laboratory

Subjects and Methods:

Patient clinical data and mRNA expression levels of MMP genes were downloaded from TCGA data portal. RACPP provides complementary ratiometric fluorescent contrast (increased Cy5 and decreased Cy7 intensities) when cleaved by MMP2/9. HNSCC-tumor bearing mice were imaged in-vivo after RACPP injection. Histology was evaluated by a pathologist blinded to experimental conditions. Zymography confirmed MMP-2/9 activity in xenografts. RACPP was applied to homogenized human HNSCC specimens and ratiometric fluorescent signal was measured on a microplate reader for ex-vivo analysis.

Results:

Expression of multiple MMPs including MMP2/9 is greater in patient HNSCC tumors than matched control tissue. In patients with human papilloma virus positive (HPV+) tumors, higher MMP2 and MMP14 expression correlates with worse 5-year survival. Orthotopic tongue HNSCC xenografts showed excellent ratiometric fluorescent labeling with MMP2/9-cleavable RACPP(sensitivity=95.4%, specificity=95.0%). Fluorescence ratios were greater in areas of higher tumor burden(p<0.03), which is useful for intraoperative margin assessment. Ex-vivo, human HNSCC specimens showed greater cleavage of RACPP when compared to control tissue(p=0.009).

Conclusions:

Human HNSCC tumors show increased mRNA expression of multiple MMPs including MMP2/9. RACPP, a ratiometric fluorescence assay of MMP2/9 activity, was used to show improved occult tumor identification and margin clearance. Ex-vivo assays using RACPP in biopsy specimens may identify patients who will benefit from intraoperative RACPP use.

### Introduction

Head and neck squamous cell carcinoma (HNSCC) is the sixth most common cancer worldwide with an estimated annual burden of 355,000 deaths and 633,000 incident cases¹. Major risk factors include smoking, alcohol abuse, and human papilloma virus (HPV)². Surgical management is usually the primary therapy for this disease, although radiation and chemotherapy also have prominent roles³.

For HNSCC, MMP expression has been shown to have prognostic value⁴⁻⁸. Of the various MMPs thought to be involved in cancer, attention has focused on MMPs 2 and 9 because they are overexpressed in a variety of malignant tumors and their expression is often associated with tumor grade and poor patient prognosis. Absolute levels of MMP2/9 have been used to differentiate between benign papillomas and carcinoma of the larynx⁴. Increased MMP2/9 expression has also been shown to correlate with cancer grade⁵ and decreased survival^{6,7}. In carcinoma of the tongue, increased MMP2/9 expression has been shown to correlate with an increased incidence of lymph node metastases⁸.

In this example, MMP mRNA levels were examined in HNSCC using The Cancer Genomic Atlas (TCGA), the largest available collection of HNSCC specimens. The prognostic value of MMP mRNA levels in patients with HPV+ and HPV−HNSCC tumors was also evaluated.

Although MMP expression (mRNA and protein) has been associated with tumor grade and poor patient prognosis for a variety of cancers, at the tissue level, MMP activity is regulated by a variety of factors including activation from pro-enzyme form and presence or absence of inhibitors⁹. Consequently MMP activity, rather than expression, may have closer association with tumor biological behavior and therefore greater prognostic value. Activatable cell penetrating peptides (ACPPs), which rely on tumor-associated proteases MMP2/9 to unmask the adhesiveness of CPPs have been previously described^{10,11}. A ratiometric version of ACPPs (RACPPS) which employs Cy5 as a far-red fluorescent resonance energy transfer (FRET) donor and Cy7 as near-infrared FRET acceptor has been recently described. The Cy5 emission is absorbed by Cy7 and re-emitted as near-infrared fluorescence until the intervening linker is cleaved by tumor-associated MMP2/9. This cleavage event increases Cy5:Cy7 emission ratio up to 40-fold and enables tissue retention of the Cy5 fragment¹². ACPP has been previously used to improve tumor margin (defined as tumor cells present at the cut edge of the surgical specimen) detection in animal model of melanoma and breast cancer¹³.

In HNSCC, positive margins have been associated with increased local recurrence and a poor prognosis¹⁴. For the majority of solid tumors, salvage surgery or adjuvant therapy not only cause extra trauma and expense but also often fail to remediate the poor outcome¹⁴⁻²⁰. The reason for this observation is likely multifactorial and related in part to the difficulty in identifying the residual cancer during repeat surgery. Therefore, development of more sensitive imaging for accurate detection of positive surgical margins during the primary operation would be one of the most effective means to prevent positive margins, thereby minimizing patient suffering and expense, while improving outcomes.

Using RACPP, MMP2/9 activity levels were compared between patient derived ex-vivo HNSCC specimens versus non-tumor tissue. The use of intravenously applied RACPPs was also evaluated to distinguish between orthotopic HNSCC xenografts from normal tissue and stratify tumor burden at the surgical margin in mice.

### Methods

All animal studies were approved by the UCSD Institutional Animal Care and Use Committee. All studies involving tumor samples obtained from HNSCC patients were approved by the UCSD Institutional Review Board.

### The Cancer Genomic Atlas (TCGA)

All available clinical and RNA expression data were downloaded from the TCGA data-portal on Dec. 15, 2013. HPV status was obtained from the TCGA HNSCC working group. HPV status was extracted from sequencing data or RNA data²¹. For tumor-normal comparison, 37 patients (out of 377 total) with matched tumor/normal tissue were considered and paired tests were used.

### Ex-Vivo Assay on HNSCC

Tumor samples were obtained from patients undergoing surgery for mucosal head and neck squamous cell carcinoma and stored at −80° C. until analysis. Samples were homogenized using equal quantities of beads and tissue and twice the volume of PBS. 150 nmol of RACPP was added to 100-175 μl of PBS containing 25 μl of 10% tissue extract. Cleavage of the probe was determined by capturing the Cy5/Cy7 fluorescence ratio every 15 minutes for 2 hours (excitation 630 nm/emission 680-780 nm) using Tecan Infinite M100 pro plate reader (Tecan Laboratories, Switzerland).

### Zymogram

Zymogram was prepared as previously described²². Briefly, 30-40 mg of tissue was homogenized in buffered solution and centrifuged. Tissue samples, along with SeeBlue Plus 2 Protein ladder and MMP standards were loaded on the gel and run at 120V for two hours. Following renaturation, development and staining, gels were imaged and analyzed with Image J. MMP activity of samples was recorded as a percentage of MMP activity within the positive control lane.

### Peptide Synthesis

RACPP and uncleavable-control were synthesized as previously described¹². The RACPP contains a poly-cationic moiety linked to a neutralizing poly-anionic arm via a linker that is cleavable by MMP-2 and MMP-9. A Cy5 fluorophore is attached to the polycationic portion while the Cy7 fluorescent molecule is attached to the poly-anionic domain. Following cleavage by MMPs, the polycationic portion conjugated to Cy5 is dequenched and becomes trapped within nearby tissue. Uncleavable-control peptide lacks an MMP cleavable linker.

### Cell Culture and Mouse Tongue Xenografts

Human tongue squamous cell carcinoma lines SCC-4, SCC-9, SCC-15, and SCC-25 (ATCC) were maintained in Dulbecco's modified Eagle's medium with nutrient mixture F-12 (DMEM/F-12) containing 10% fetal bovine serum (FBS) and supplemented with 400 ng/mL of hydrocortisone. Human tongue squamous cell carcinoma line CAL-27 (ATCC) was maintained in DMEM containing 10% FBS. Cells were incubated at 37° C. in 5% CO₂. Nu/nu mice (age, 3-6 months) were injected with cultured HNSCC cells (˜10⁶ for CAL-27, ˜5×10⁶ for SCC-4, SCC-9, SCC-15, SCC-25) into the tip of the tongue. One cell line was used in each mouse for these experiments (n=22 total; CAL-27:n=5, SCC-25 n=4, SCC-15:n=4, SCC-4:n=4, SCC-9:n=5).

In Vivo Imaging with RACPP

Mice were monitored for 20% weight loss or tumor size >4-5 mm. Once these parameters were met, animals were anesthetized with isoflurane and injected intravenously with RACPP or control uncleavable peptide (0.4 nmol/g). Two hours after injection, mice were re-anesthetized (100 mg/kg ketamine and 5 mg/kg midazolam) and subcutaneous cervical tissue/anterior tongue exposed for imaging (Maestro, CRI). After completion of whole body imaging, animals were euthanized. The entirety of the tongue was immediately extracted and imaged in the dorsal position (Maestro, CRI).

Spectral imaging was carried out by exciting Cy5 at 620 (±10) nm followed by step-wise emission measurements from 640 to 840 nm through a tunable LCD emission filter. For ratio imaging, numerator (Cy5) and denominator (Cy7) images were generated by integrating spectral images over a defined range at 10 nm intervals (660-720 nm for Cy5 and 760-830 nm for Cy7). Ratio images were generated and color-encoded using custom software. The ratio for each pixel was encoded as hue on a blue to red scale and brightness was based on the original Cy5 images. The software also generated monochromatic Cy5/Cy7 images for further processing (see Image analysis and histologic correlation).

### Histology

Immediately following imaging, tongue tissues were embedded in cryopreservative and stored at −80° C. Samples were cryosectioned into 5-μM sections in the same orientation as the whole tongue molecular imaging and stained with hematoxylin and eosin (H&E). The entirety of the tongue was included in the slice, including both tumor and normal tissue. Samples were evaluated by a pathologist blinded to experimental conditions.

### Mapping Histology to Molecular Imaging

For histologic samples, a pathologist blinded to experimental conditions used a stage micrometer to determine the tumor's linear position and extent along the length of the tongue. This information was mapped to spectral images of the tongue. A mean Cy5/Cy7 ratio was calculated for segments containing histologically-confirmed tumor and, separately, tumor-free segments.

Percent tumor involvement was approximated by the pathologist as the density of cancerous tissue (vs. non-cancerous tissue) within the tumor-containing segment of tongue. For example, if the tumor-bearing length of the sample contained only malignant cells and no normal tissue, percent involvement was recorded as 100%. If only half of this region contained malignancy, percent involvement was recorded as 50%. This method for calculating percent tumor involvement has been utilized in other studies^{23,24}.

### Image Processing and Ratio Calculations

Monochromatic Cy5 and Cy7 images were extracted from the spectral image using custom software. Using the “Image Calculator” feature on Image J, the Cy5 image was divided by the Cy7 image to produce a new image, where Cy5/Cy7 ratios were encoded by pixel intensity. Ratios were calculated separately for tumor and normal tongue, which were distinguished based on the histologic map described above. These ratios were each normalized to Cy5/Cy7 ratios of background tissue (cervical soft tissue).

### Statistics

Statistical analysis between experimental groups was conducted using either the 2-tailed independent sample student t test or one-way ANOVA w/post-hoc analysis. Graphical bar-plots were produced using Microsoft Excel, while ROC curves were created with Sigmaplot (12.3). Paired tests were used for TCGA analysis due to matched expression data. For survival analysis, Cox proportional hazards regression was employed using the R ‘survival’ package.

### Results

### MMPs are Overexpressed in HNSCC

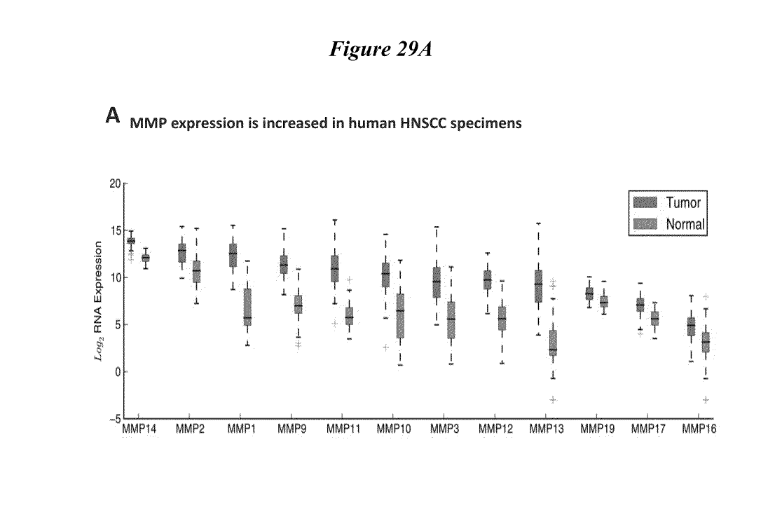

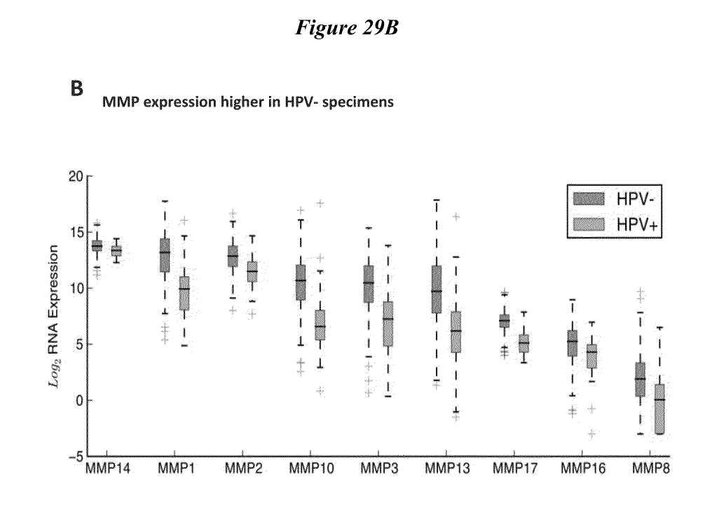

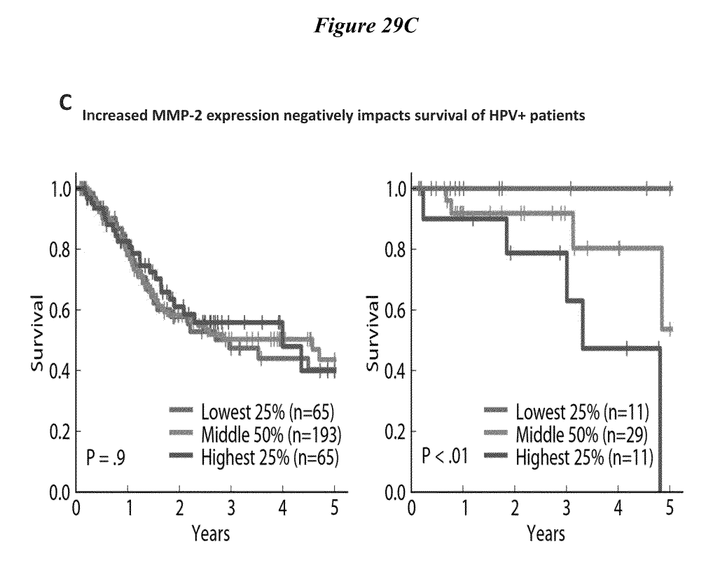

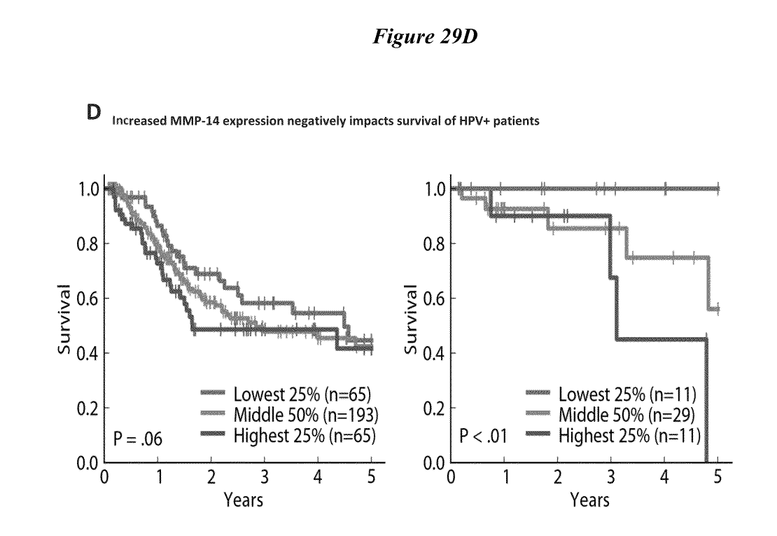

To evaluate MMP expression levels in HNSCC from the TCGA, patient-derived tumor specimens were compared with matched normal control tissue. TCGA profiled matched normal tissue for approximately 10% of the patients (37 of 377). Thus, this data was used in our analysis. HNSCC tumors showed increased expression of multiple MMPs compared to matched control non-tumor tissue (FIG. 29A, all p values <0.01). Interestingly, MMP14 (also known as MT1-MMP) was the protease with the highest total expression in tumor tissue and had significantly higher expression in tumor compared to matched control tissue (p<10⁻⁵). The second highest expressing MMP in tumor tissue was MMP2. MMP2 and 9 share a common cleavage sequence and they have been particularly well characterized in prior studies in association with HNSCC²⁵. It was found that both MMP2 (p<10⁻¹⁰) and MMP9 (p<10⁻⁶) have significantly greater RNA expression in HNSCC tumors compared to paired-control tissue (n=37, (34 HPV− and 3 HPV+), Wilcoxon signed-rank test).

### MMP 2 and 14 Stratify Survival in HPV+HNSCC

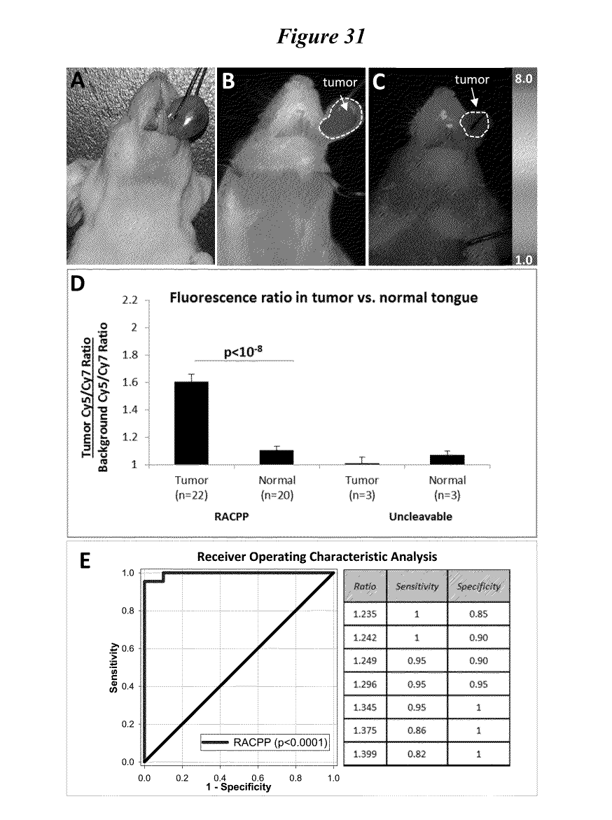

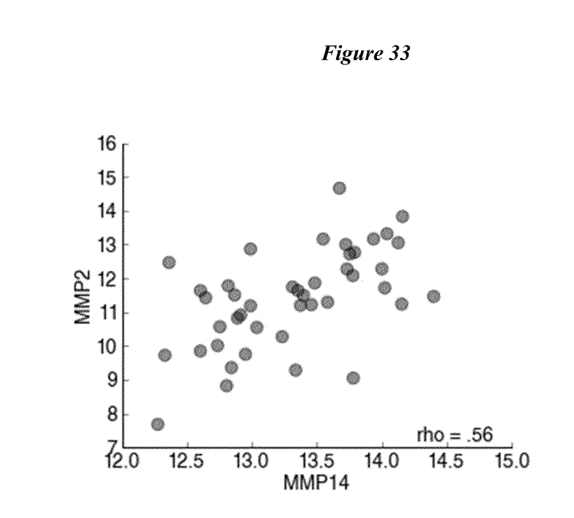

Next, the difference in MMP expression between HPV+ and HPV− tumors was evaluated. It was found that HPV+ tumors had less overall MMP expression compared to HPV− tumors (FIG. 29B, Kruskal-Wallis test on pooled RNA levels, p<10⁻¹⁰). This is consistent with the hypothesis that HPV+ tumors are less biologically aggressive, and consequently, that these patients tend to have improved survival compared to patients with HPV− tumors. Interestingly, it was found that in patients with HPV+HNSCC, increased expression levels of MMP2 and MMP14 correlated with worse survival (FIG. 29C, D p<0.01). Patients with HPV+ tumors who have the highest MMP2 and MMP14 expression (FIG. 31C, D red lines) had significantly worse 5 year survival compared to patients with the lowest expression levels of these proteases (FIG. 29C, D blue lines). Additionally, for a given patient with HPV+ tumor, there is a significant correlation between MMP2 and MMP14 expression (FIG. 33, Spearman Rho=0.56, p<10⁴). Thus, poor prognosis HPV+ tumors stratified in the highest quartiles of MMP2 expression are also likely to have higher expression of MMP14. The same correlation in MMP expression with survival in patients with HPV− tumors was not found. The cause of this is multifactorial and likely related to the observation that HPV-tumors have more genetic mutations compared HPV+ tumors²⁶.

### Zymography

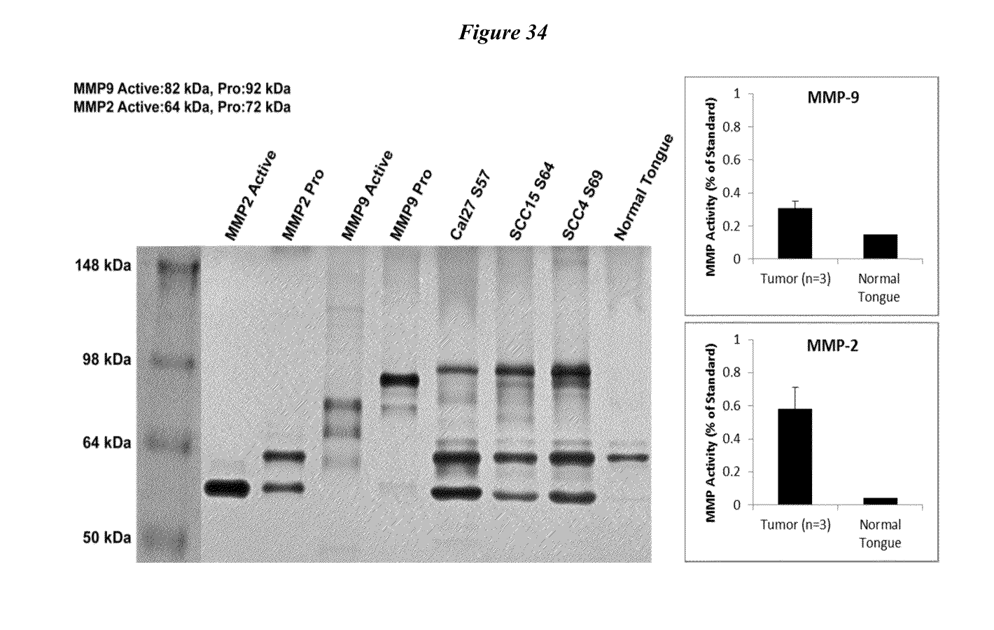

To confirm MMP2/9 activity in mouse HNSCC xenografts, cleavage of gelatin by tumor homogenates was measured via zymography. A two-fold increase in MMP9 and a 13-fold increase in MMP2 activity in HNSCC xenografts compared to normal mouse tongue tissue was found (FIG. 34).

### RACPP in Ex-Vivo HNSCC

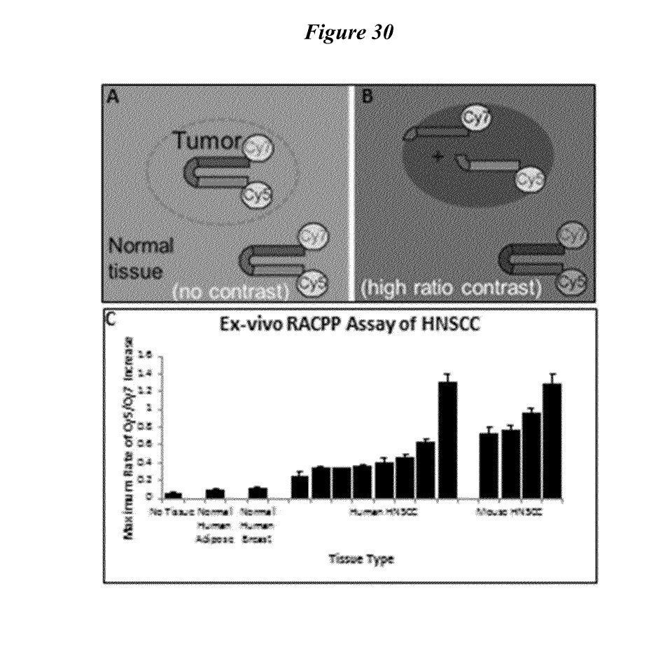

To evaluate ex-vivo MMP2/9 activity in human and mouse HNSCC specimens, the maximum rate of Cy5/Cy7 ratio change over time in homogenates following addition of RACPP was measured (FIG. 30A, B). It was found that patient derived HNSCC specimens show higher MMP2/9 activity compared to non-tumor tissue (FIG. 30C) (ROC Analysis: AUC=1.000, p=0.01). Similarly, mouse HNSCC xenografts also show higher Cy5/Cy7 rate change, signifying higher MMP2/9 activity compared to non-tumor tissue (ROC Analysis: AUC=1.000, p=0.03).

### RACPP Improves Detection of HNSCC

To test tumor-dependent Cy5/Cy7 ratiometric change in living mice, tongue tumor bearing nu/nu mice were intravenously injected with RACPP (n=25). Multispectral imaging of these live, anesthetized mice (ex 620, em 640-840 nm, Maestro, CRI at 2 hours after injection) with both tongue and subcutaneous cervical tissue exposed was conducted (FIG. 31A). The tongue was excised and multispectral imaging of the tongue performed. Histologic information regarding tumor location and size was correlated and mapped to ratiometric fluorescence image of the tongue. Sample (tumor and non tumor tissue) Cy5/Cy7 ratios were divided by “background” subcutaneous cervical tissue Cy5/Cy7 ratio to compute a “normalized Cy5/Cy7 ratio”¹².

It was found higher Cy5/Cy7 ratiometric fluorescence in tumor (FIG. 3B, red color) compared to adjacent normal tongue (FIG. 31B, tan color). Injections of our control (uncleavable) probe revealed no ratiometric difference between tongue tumor and normal tongue (FIG. 31C). Following intravenous administration of MMP2/9-cleavable RACPP, mice showed greater normalized Cy5/Cy7 ratio in tumor (1.61±0.05, n=22) compared to normal tongue (1.11±0.03, n=20, p<10⁻⁸). This increase in ratiometric fluorescence in orthotopic tumors was not seen following intravenous injection of uncleavable control probe (tumor=1.01±0.04, n=3; normal tongue=1.07±0.03, n=3, p=0.30) (FIG. 31D).

The receiver-operating curve (ROC) for cleavable RACPP revealed an area under the curve (AUC) of 0.995±0.006 (p<10⁻⁴) with a peak sensitivity of 95% and peak specificity of 100% for a normalized ratio cutoff of 1.345 (FIG. 31D insert). The two tumor specimens not detected by this threshold-cutoff had relatively low tumor burden (<60% involvement, see below).

### RACPPs Enable Stratification of Tumor Burden

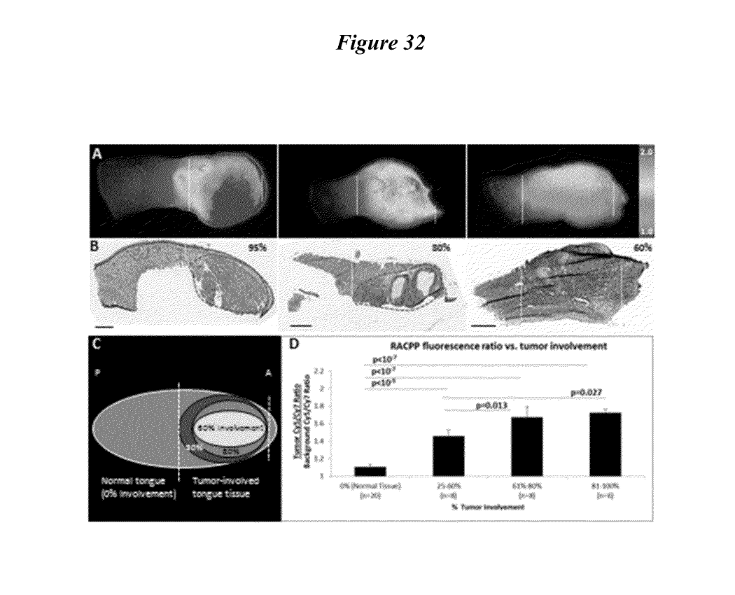

One critical component of intraoperative margin evaluation is determining how much tumor burden is present at the edges of the surgical field. To evaluate the ability of RACPP to stratify tissue with variable tumor burden, percent involvement of cancer within tumor-bearing portions of each sample was approximated by a pathologist blinded to experimental conditions. It was found that varying levels of tumor burden among the 22 samples ranging from 25-100% invasion (FIG. 32A). To evaluate the stratification of ratiometric fluorescence values between different levels of tumor burden, samples were statistically separated into the following tertiles of cancer involvement: 25-60% (n=8), 61-80% (n=8), 81-100% (n=6) (FIG. 32B). Adjusted Cy5/Cy7 ratios were computed for each tertile and compared with normal tongue tissue (n=20).

It was found that all tertiles of varying tumor burden showed significantly greater normalized Cy5/Cy7 ratio than normal tongue tissue (FIG. 32C; lowest tertile of tumor involvement=1.46±0.07, p<10⁻⁵, middle tertile=1.67±0.12, p<10⁻⁷, highest tertile=1.72±0.04, p<10⁻⁷). Additionally, tumors with percent involvement in the highest and middle tertiles showed significantly greater normalized ratios than the lowest tertile (p=0.01 for highest vs. lowest tertile, p=0.03 for middle vs. lowest tertile). Future experiments will focus on evaluating the ability of RACPP to detect incrementally smaller levels of tumor burden (i.e. from 1-25% involvement).

### Discussion

In this example, mRNA expression levels for MMPs in human HNSCC were analyzed using The Cancer Genomic Atlas (TCGA), the largest available collection of human HNSCC specimens. The prognostic value of MMP overexpression in terms of survival in patients with HPV+ and HPV− HNSCC tumors was also evaluated. It was found that many MMPs are overexpressed in HNSCC tumors compared to paired control tissue. However, patients with HPV+HNSCC tumors have significantly lower overall MMP levels compared to patients with HPV− HNSCC tumors. This finding is consistent with previous studies showing that patients with HPV+HNSCC tumors have better overall survival compared to patients with HPV− HNSCC tumors²⁷.

Of the various MMPs thought to be involved in cancer, attention has focused on MMP2/9 because they are overexpressed in a variety of malignant tumors and their expression is often associated with tumor grade and poor patient prognosis. Interestingly, it was found that of the MMPs that are increased in tumor compared to control tissue, MMP2 and MMP14 are expressed at higher levels compared to all other MMPs, suggesting that these proteinases may be particularly important in HNSCC. Furthermore, it was found that in patients with HPV+HNSCC tumors, increased MMP2 and MMP14 expression levels correlated with worse overall survival. If clinically validated in a prospective trial, the increases in MMP2 and MMP14 represent two molecular biomarkers that can individualize management of patients with HPV+ tumors.

Using MMP2/9 cleavable RACPP, It was found that ex-vivo human HNSCC specimens show greater activity compared to normal tissue. This finding correlates with previous studies demonstrating higher MMP2/9 expression at the invasive edge of tumors²⁸. The high sensitivity and specificity of RACPPs to differentiate between tumor and normal tissue suggests that ex-vivo measurements of MMP2/9 activity in HNSCC specimens may complement MMP mRNA expression studies in evaluating patient prognosis and in determining which patients would benefit from RACPP guided surgery.

In multiple human cell line models of HNSCC xenografts, it was found that higher MMP2/9 activity as evidenced by gelatinase zymography and higher ratiometric fluorescence signal following systemically applied RACPP compared to non-tumor tissue. All ratios were computed from histologically confirmed tumor or normal tissue, eliminating verification bias. The ideal discrimination threshold for detecting cancer versus normal tissue is 1.345, which is consistent with previously reported ratiometric thresholds for this probe¹². Our study tested multiple tongue squamous cell carcinoma cell lines from ATCC to highlight the RACPP's broad applicability.

One critical component of intraoperative margin evaluation is determining how much tumor burden is present at the edges of the surgical field. It was found that within tumor bearing tissue, the greater the tumor burden, the greater the ratiometric fluorescence signal following intravenous RACPP administration. Percent tumor involvement has been shown to be important for survival and recurrence outcomes in prostate and breast cancer²³′²⁹. The correlation between intraoperative ratiometric fluorescence level and tumor burden suggests that RACPP can improve intraoperative decision making by providing information regarding local level of tumor involvement and consequently margin clearance.

### REFERENCES FOR EXAMPLE 5

1. Ferlay J, Shin H R, Bray F, Forman D, Mathers C, Parkin D M. Estimates of worldwide burden of cancer in 2008: GLOBOCAN 2008. Int J Cancer 2010; 127:2893-2917.
2. D'Souza G, Kreimer A R, Viscidi R et al. Case-control study of human papillomavirus and oropharyngeal cancer. N Engl J Med 2007; 356:1944-1956.
3. Shin D M, Khuri F R. Advances in the management of recurrent or metastatic squamous cell carcinoma of the head and neck. Head Neck 2013; 35:443-453.
4. Uloza V, Liutkevicius V, Pangonyte D, Saferis V, Lesauskaite V. Expression of matrix metalloproteinases (MMP-2 and MMP-9) in recurrent respiratory papillomas and laryngeal carcinoma: clinical and morphological parallels. Eur Arch Otorhinolaryngol 2011; 268:871-878.
5. Wittekindt C, Jovanovic N, Guntinas-Lichius O. Expression of matrix metalloproteinase-9 (MMP-9) and blood vessel density in laryngeal squamous cell carcinomas. Acta Otolaryngol 2011; 131:101-106.
6. Liu W W, Zeng Z Y, Wu Q L, Hou J H, Chen Y Y. Overexpression of MMP-2 in laryngeal squamous cell carcinoma: a potential indicator for poor prognosis. Otolaryngol Head Neck Surg 2005; 132:395-400.
7. Mallis A, Teymoortash A, Mastronikolis N S, Werner J A, Papadas T A. MMP-2 expression in 102 patients with glottic laryngeal cancer. Eur Arch Otorhinolaryngol 2012; 269:639-642.
8. Zhou C X, Gao Y, Johnson N W, Gao J. Immunoexpression of matrix metalloproteinase-2 and matrix metalloproteinase-9 in the metastasis of squamous cell carcinoma of the human tongue. Aust Dent J 2010; 55:385-389.
9. Chaudhary A K, Singh M, Bharti A C, et al. Genetic polymorphisms of matrix metalloproteinases and their inhibitors in potentially malignant and malignant lesions of the head and neck. J Biomed Sci 2010; 17:10.
10. Olson E S, Aguilera T A, Jiang T et al. In vivo characterization of activatable cell penetrating peptides for targeting protease activity in cancer. Integr Biol (Camb) 2009; 1:382-393.
11. Aguilera T A, Olson E S, Timmers M M, Jiang T, Tsien R Y. Systemic in vivo distribution of activatable cell penetrating peptides is superior to cell penetrating peptides. Integr Biol 2009; 1:371-381.
12. Savariar E N, Felsen C N, Nashi N et al. Real-time in vivo molecular detection of primary tumors and metastases with ratiometric activatable cell-penetrating peptides. Cancer Res 2013; 73:855-864.
13. Savariar E N, Felsen C N, Nashi N et al. Real-time in vivo molecular detection of primary tumors and metastases with ratiometric activatable cell-penetrating peptides. Cancer Res 2013; 73:855-864.
14. Hague R, Contreras R, McNicoll M P, Eckberg E C, Petitti D B. Surgical margins and survival after head and neck cancer surger. BMC Ear Nose Throat Disord 2006; 16:2.
15. Singletary S. Surgical margins in patients with early-stage breast cancer treated with breast conservation therapy. Am J Surg 2002; 184:383-393.
16. Nagtegaal I D, Quirke P. What is the role for the circumferential margin in the modern treatment of rectal cancer? J Clin On 2008; 26:303-312.
17. Meric F, Mirza N, Vlastos G et. al. Positive surgical margins and ipsilateral breast tumor recurrence predict disease-specific survival after breast-conserving therapy. Cancer 2003; 97:926-933.
18. Snijder R, de la Riviere A, Elbers H, van den Bosch J. Survival in resected stage I lung cancer with residual tumor at the bronchial resection margin. Annals of Thoracic Surg 1998; 65.
19. Dotan Z, Kavanagh K, Yossepowitch 0 et al. Positive surgical margins in soft tissue following radical cystectomy for bladder cancer and cancer specific survival. J Urol 2007; 178:2308-2312.
20. Wieder J A, Soloway M S. Incidence, etiology, location, prevention and treatment of positive surgical margins after radical prostatectomy for prostate cancer. J Urol 1998; 160:299-315.
21. Gross A, Orosco R, Shen J et. al. A prognostic model of head and neck cancer ties TP53 mutation to 3p loss. Nature Genetics [Under Review].
22. Toth M, Sohail A, Fridman R. Assessment of gelatinases (MMP-2 and MMP-9) by gelatin zymography. Methods Mol Biol 2012; 878:121-35.
23. Rampersaud E N, Sun L, Moul J W, Madden J, Freedland S J. Percent tumor involvement and risk of biochemical progression after radical prostatectomy. J Urol 2008; 180:571-576; discussion 576.
24. Thompson I M, 3rd, Salem S, Chang S S et al. Tumor volume as a predictor of adverse pathologic features and biochemical recurrence (BCR) in radical prostatectomy specimens: a tale of two methods. World J Urol 2011; 29:15-20.

25. Xia T, Akers K, Eisen A Z, Seltzer J L. Comparison of cleavage site specificity of gelatinases A and B using collagenous peptides. Biochim Biophys Acta 1996; 1293(2):259-266.
26. Maruyama H, Yasui T, Ishikawa-Fujiwara T, et al. Human papillomavirus and p53 mutations in head and neck squamous cell carcinoma among Japanese population. Cancer Sci 2014. [In Press]
27. Sivars L, Nasman A, Tertipis N et al. Human papillomavirus and p53 expression in cancer of unknown primary in the head and neck region in relation to clinical outcome. Cancer Med 2014; 3(2): 376-84.
28. Kuniyasu H, Troncoso P, Johnston D et al. Relative expression of type IV collagenase, E-cadherin, and vascular endothelial growth factor/vascular permeability factor in prostatectomy specimens distinguishes organ-confined from pathologically advanced prostate cancers. Clin Cancer Res 2000; 6(6): 2295-308.
29. Kohrt H E, Noun N, Nowels K, Johnson D, Holmes S, Lee P P. Profile of immune cells in axillary lymph nodes predicts disease-free survival in breast cancer. PLoS Med 2005; 2:e284.

### Example 6

### A. Significance

### A1.

Multiple proteases have been evaluated for their roles in cancer growth, invasion and metastasis, including matrix metalloproteinases (MMPs)¹ and urokinase plasminogen activator (uPA), cathepsins, interstitial collagenase (aka MMP1), elastases, ². MMPs are a class of endopeptidases that breakdown extracellular matrix leading to localized inflammation and tissue permeability both of which are associated with tumorigenesis and metastasis. Broad inhibition of MMPs for the treatment of advanced cancer has been unsuccessful in clinical trials³. It is now recognized that MMPs can have both inhibitory and stimulatory effects on tumor progression^{4,5}, thus a better understanding of the in vivo activity of specific MMPs in the context of cancer is needed to develop effective therapies or imaging agents. MMP2 and 9 are two very well studied gelatinases that can degrade collagen in the basement membrane which is postulated to be necessary for angiogenesis and metastasis⁶. Also the inflammatory microenvironment within tumors causes upregulation of MMP2 and 9 via MMP14 activation leading to invasion in intestinal cancer⁷. MMP14 (also known as MT1-MMP) is a membrane-tethered active protein that accumulate in invadopodia-like structures on the cell membrane to allow the cells to tunnel through the surrounding matrix. Inhibition of MMP14 expression with RNA interference had no effect on triple negative breast cancer cell growth but significantly diminished the number of migrating tumor cells and the incidence of lung metastasis⁹.

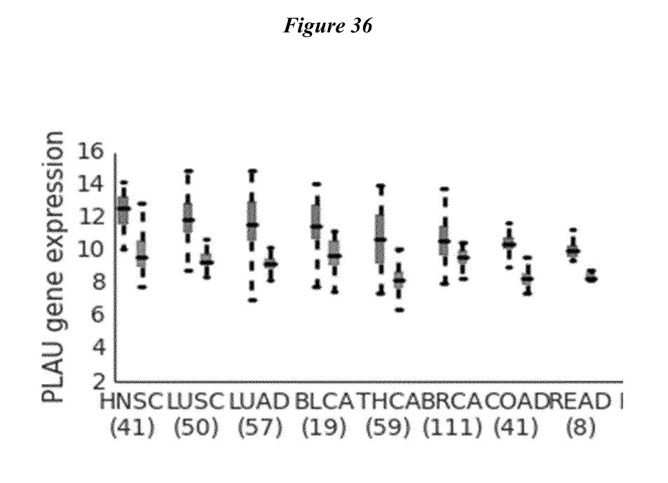

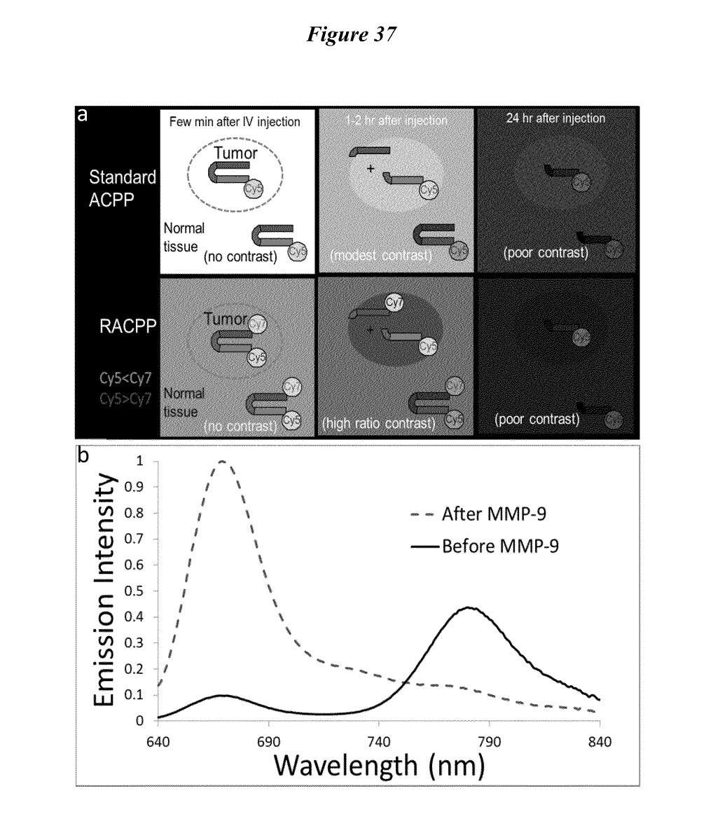

Although MMP2,9 are also increased in inflammation/wound healing, absolute levels of these gelatinases in the head and neck have been used to differentiate between benign papillomas versus carcinoma of the larynx¹⁰ Increased MMP2,9 expression has been shown to correlate with cancer grade¹¹ and decreased survival^{12,13}. In carcinoma of the tongue, increased MMP2,9 expression has been shown to correlate with incidence of lymph node metastases¹⁴. From our own TCGA data analysis, it was found that all cancers, including HNSCCs have significantly higher expression of many MMPs compared to match normal tissue (particularly MMP14, MMP1, MMP2, MMP9; FIG. 36)¹⁵. It was also found that tumors from patients with HPV− HNSCC (which have poor prognosis compared to HPV+HNSCC) have significantly higher MMP expression levels compared to tumors from patients with HPV+HNSCC¹⁵. Interestingly, it was also found that in patients with HPV+HNSCC, increased MMP2 and MMP14 expression correlated with worse survival¹⁵, again, consistent with the hypothesis and previous reports of a correlation between increased MMP levels and worse prognosis.

In addition to the well-studied role of MMPs, plasminogen activation is also believed to be critical in the progression of multiple human cancers by facilitating matrix degradation during invasion and metastasis¹⁶. Urokinase plasminogen activator (uPA) levels as measured by zymography has been shown be highly increased in tumor compared to adjacent normal tissue². From our own TCGA data analysis, it was also found that uPA mRNA expression is highly increased in tumor compared to paired normal tissue for multiple cancers including HNC (FIG. 36, Gross et al, unpublished data). In contrast, mRNA expression levels of tissue plaminogen activator (tPA), elastases and cathepsins are not significantly increased in the TCGA tumor specimens (data not shown, Gross et al, unpublished).

### A3. Ratiometric Cell Penetrating Peptides (RACPP)s

Ratiometric activable cell penetrating peptides (RACPP, FIG. 36) which are protease sensitive molecules that undergo a change in the Cy5-Cy7 fluorescence intensity ratio and localized retention upon cleavage by MMPs that are upregulated on the surface of many tumor tissues have been previously described¹⁷. In contrast to mRNA expression or copy number detection methods, RACPPs detect in vivo protease activity, thus bypassing the complicated interplay between transcription/translation, pro- and active forms of the enzymes, as well as the presence of inhibitors/activators etc. It was shown that RACPP uptake correlates with tumor burden¹⁵ and with tumor characteristics corresponding to poor survival¹⁸. This class of molecule has been used intraoperatively to demonstrate improved tumor free survival following surgery in breast cancer, melanoma¹⁹ salivary gland cancer²⁰ and pancreatic adenocarcinoma²¹. It the also demonstrated decreased tumor burden following delivery of targeted chemotherapeutics²² and radiosensitizer²³ in animal models. Although this first generation molecule promises wide applicability for use in surgical resection of multiple types of solid tumors and is being tested in an FDA Phase 1b clinical trial, one disadvantage is that the current cleavable component of the molecule (PLGC(Me)AG) (SEQ ID NO: 2) is cut by multiple MMPs, limiting signal to noise resolution and potentially limiting sensitivity/specificity of the probe to precisely define tumor boundaries.

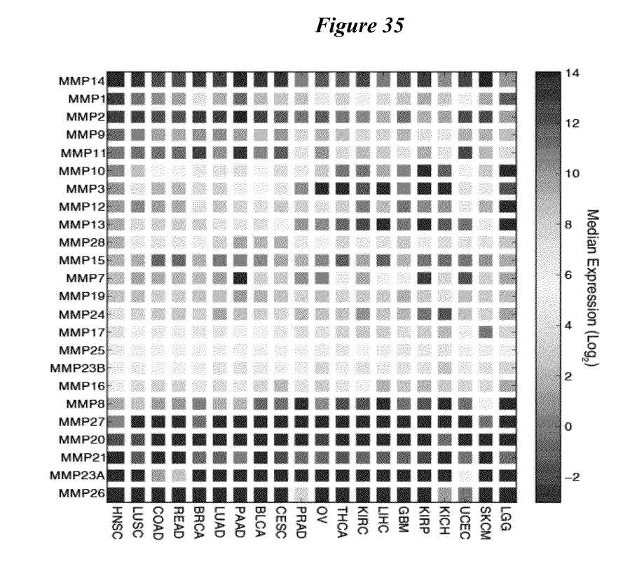

Literature review and our own recent analysis of TCGA data showed that mRNA expression of several proteases are selectively increased in tumor compared to normal tissue in patient-derived samples (FIG. 35, 36)^{2,24}. In this example, rational design will be used to improve selectivity of the cleavable component by the tumor selective proteases (MMP14, 1, 2, 9, 11 and uPA) in human HNC. The sensitivity/specificity of novel individual protease-selective RACPPs for HNC tumor detection and compare tumor free survival following surgery with and without protease-cleavable RACPP fluorescence guidance will be determined.

Radiotherapy is a mainstay treatment modality for HNC, as definitive therapy or postoperative adjuvant. Intensity-modulated-radiation-therapy (IMRT) has the benefit of reducing morbidity through highly conformal ionizing radiation delivery²⁵. In the second part of this example, it will be tested whether uptake of these novel protease-selective RACPPs can be exploited to modulate tissue sensitivity to radiotherapy with the intent to spare adjacent tissue injury without compromising and potentially enhancing tumor control. The novel protease-selective ACPPs will be linked with known radiosensitizers and evaluate tumor control following dual action radiotherapy.

### B. Innovation

There are four main areas of innovation in this proposal. The first one is the use of TCGA for direct identification of clinically relevant proteases for a specific type of cancer (in this case head and neck cancer). Several candidate proteases which show the highest mRNA expression (MMP14, aka MT1-MMP, MMP1, 2, 9, 11 and uPA) in tumor specimens compared to paired normal tissue were identified¹⁵. Rational strategies²⁶ to design the cleavable site to provide selectivity for our molecularly targeted agents were used.

The second area of innovation is the use of TCGA to direct preclinical tumor model classification for use in this study. Using TCGA, it was found that patients whose tumors have combined TP53 mutation and 3p deletion (double-hit) have significantly poorer clinical outcome compared to patients with either events alone (single-hit)²⁴. Multiple readily available cell lines that mirror these classifications have been identified¹⁸. This double/single-hit classification for tumor xenografts and spontaneous oral carcinogen-derived models will be used in this example to evaluate whether or not differential protease activity and sensitivity to ionizing radiation mechanistically contribute to this difference in clinical outcome.

A third area of innovation is the use of ratiometric fluorescence guided molecular imaging during surgery to evaluate protease activity at the advancing tumor edge vs. metastatic lymph nodes¹⁷. The advantages of ratiometric vs. single-intensity measurements are well known²⁷ in fluorescence microscopy and flow cytometry and are leveraged here for intraoperative molecular imaging. It is hypothesized that high levels of specificity and sensitivity for a given protease-specific probe at tumor edges or metastatic lymph node will result in high signal to noise ratio required for potential clinical translation. In vivo fluorescence imaging will be used with molecular navigation to guide surgical resection of tumors and compare tumor free survival with surgery using protease-selective cleavable probes vs surgery using white light reflectance alone in HNC, building on our expertise gained during our prior studies in multiple other solid tumors, including breast, melanoma, salivary gland carcinoma²⁰ and pancreatic adenocarcinoma¹¹.

Finally, a fourth area of innovation is to use molecular targeting of protease activity for guided radiotherapy or to localize radiation sensitizers to the cells/tumors that have the highest protease levels²³. Tumors with high protease levels (MMPs, uPA) have previously been shown to correlate with highest stage, grade and metastatic potential^{2.11-13.} It is hypothesized that protease-cleavable probes may be useful for targeting advanced stage tumors and that this characteristic can be leveraged to target the highest levels of localized radiation with or without radiosensitizers to improve tumor control and decrease damage to surrounding tissue.

B1. Innovation 1a—TCGA Directed Identification of Clinically Relevant Proteases.

Although there has been significant literature documenting the correlation of protease levels with stage, grade and metastatic potential^{11,13,14} data from TCGA was used which represent the largest collection of tumors with detailed multi omics characterization to identify proteases which are most highly expressed (mRNA) for multiple cancers. One limitation of mRNA expression is that it correlates poorly with protein abundance, which further correlates poorly with enzymatic activity. As mentioned above, increased MMP14 and MMP2 mRNA did correlate with worse overall survival for patients with HPV+ tumors however this correlation was not found in patients with HPV− tumors, likely due to increased heterogeneity in the HPV− population¹⁷. This example will leverage TCGA mRNA data to develop protease sensitive probes that will report enzymatic activity which potentially has much better correlation with actual tumor biology and prognostic utility. Although the focus of this proposal is proteases in HNC¹⁵, proteases important in multiple other solid tumors that will benefit from rational design of protease-selective probes were identified.

Innovation 1b—Rational Design of Molecular Imaging Agents Specific for Select MMPs and uPA

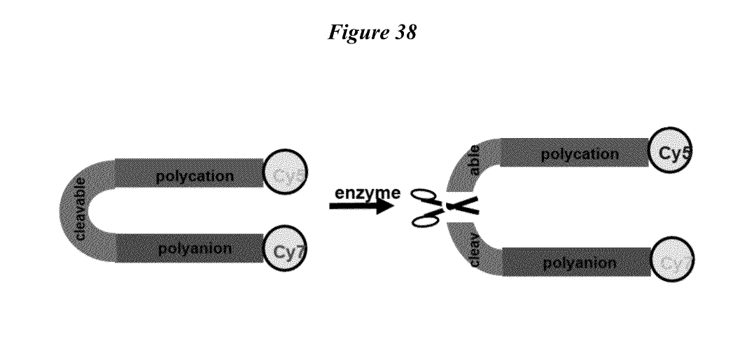

In contrast to expression detection methods for proteases, RACPPs which are capable of monitoring enzyme activity in real time in living animals have been previously described¹⁷. RACPPs are molecular targeting probes that target selective proteases by linking positively charged cell penetrating peptide (FIG. 38, blue segment) to the neutralizing negatively charged segment (FIG. 38, red segment) with a protease selective linker (FIG. 38, green segment). The emission of the Cy5 (far red) fluorescent donor is quenched in favor of Cy7 re-emission until the intervening linker is cleaved by tumor-associated proteases, which ratiometrically increases the Cy5:Cy7 emission ratio up to 40 fold and triggers tissue retention of the Cy5-containing fragment. This large change in ratio provides a wide dynamic range in which protease activity in tumors and metastases can be quantitatively differentiated from adjacent normal tissue¹⁷. Furthermore, it has been shown that RACPPs can be used as a high throughput assay to interrogate existing ex vivo patient tumor specimens to probe protease activity¹⁵ and correlate with clinical stage and outcome. This represents a significant advance compared to existing technologies measuring mRNA or protein expression which correlates poorly with enzymatic activity and limits correlation with clinical outcomes.

To generate the RACPP cleavable sites (FIG. 38, green segment) that are selective for given MMPs, previously identified substrates and recent data by Ranikov et al²⁶ who used a large set of phage peptide substrates were leveraged to interrogate cleavage rates of multiple MMPs to determine the relative impact of individual specificity determining positions (SDP). These authors identified specific substrates and consensus residues from P3 to P2′ that were highly selected and therefore likely preferred by a given MMP²⁶. In particular, there was a focus on identifying and testing specific substrates that distinguish the two gelatinases MMP2 and MMP9, and MMP14 which have similar substrate preference and were highly upregulated in head and neck tumors and most other cancers from TCGA (FIG. 35).

To generate novel RACPP species that are selective for uPA, a set of protease substrates were screened that were diverse and likely cleaved by serine protease in that they contain either a lysine or arginine residue flanked by diverse amino acids. Upon testing, one 6 amino acid substrate with sequence YGRAAA (SEQ ID NO: 17) was found to be efficiently cleaved by urokinase plasminogen activator (uPA) and to a lesser extent by tPA. Several other substrates that were reported to be cleaved by uPA were also tested but were not cleaved possible because the constrained structure of the substrate within the context of an ACPP.

B2. Innovation 2—TCGA directed tumor model classification.

In our recent analysis of TCGA HNSCC specimens, it was found that although TP53 has previously been shown to be the most commonly mutated, prognostic driver gene in HNSCC²⁸, in patients with HPV− tumors, the detrimental impact of TP53 mutation occurs) ₄ only in combination with loss of chromosome 3p²⁴. The combined TP53 mutation and 3p deletion (double-hit) led to a marked decrease in median survival from >5 years for TP53 mutation only (single-hit) to 1.7 years for both events. In the 3p region, it was found that copy number variation for Fragile Histidine Triad Protein (FHIT) correlates best with 3p deletion status. FHIT has been shown to be a tumor suppressor²⁹ and FHIT deficient mice show both increased incidence of spontaneous tumor formation as well as increased tumor formation in response to carcinogens³⁰. Loss of FHIT expression has been shown to correlate with poor outcome in patients with tongue cancer³¹. Using already available, well characterized HNC cell lines which have either the “double-hit” (Cal 27, SCC15, SCC25) or “single-hit” (SCC4) genotype, it was previously shown that “double-hit” tumors have significantly higher generalized MMP activity compared to “single-hit” tumors (using our first generation RACPP)¹⁵. This example aims to test the difference in specific protease activity (i.e. MMP14, 1, 2, 9, 11 or uPA between the two tumor types (double vs. single-hit). A parallel experiment will be to test the specific protease activity levels (i.e. MMP14, 1, 2, 9, 11 or uPA) in metastatic lymph nodes derived from these tumor types. Our hypothesis is that RACPPs that are uniquely sensitive to cleavage by single individual proteases will demonstrate improved sensitivity/specificity for differentiation tumor vs. adjacent normal tissue compared to our existing first generation PLGC(me)AG (SEQ ID NO: 2) substsrate which is cleaved by multiple proteases.

B3. Innovation 3-Molecular Imaging to Guide Surgery, Pathological Examination of Surgical Specimens and Ex Vivo Examination of Protease Activity of Banked Tissues for Correlation with Outcome

ACPPs have previously been shown to improve tumor free survival following fluorescence guided surgery for breast, melanoma, pancreatic and salivary gland cancers in animal models. Extensive experience in fluorescence guided surgery with molecular navigation to evaluate tumor-free survival with and without the use of these novel protease-selective probes in animal models of HNC will be used. One important difference between our proposed use of ratiometric fluorescence imaging with RACPP vs. other antibody or ligand-fluorescence conjugate strategies is quick wash out period of approximately 90 minutes for our agents¹⁷ compared to days for antibody based technologies^{12,32}. The quick wash out period allows RACPP agents to be administered on the day of surgery during preoperative preparations instead of bringing the patient in several days prior to surgery solely to administer the targeting agent, simplifying the clinical work flow and minimizing related costs.

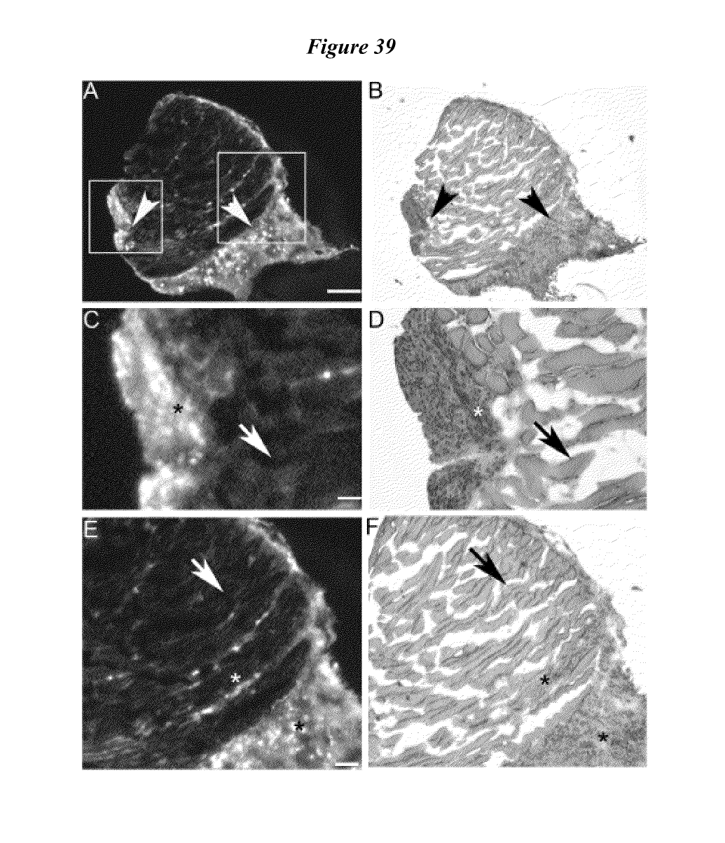

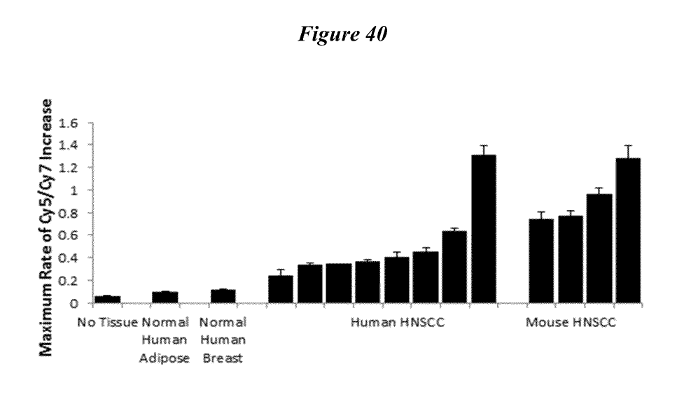

Furthermore, previous work that surgical specimens treated with intravenous RACPPs retain ratiometric fluorescence in the frozen blocks to focus ex vivo pathological examination of the tissue will be expanded (FIG. 39¹⁹), potentially improving diagnostic accuracy for eventual ex vivo clinical translation. Finally, this example aims to use protease-selective RACPPS to develop an ex vivo screening assay of protease activity in banked tissue (FIG. 40¹⁵) for correlation with stage and clinical outcome.

Although focusing on HNC animal models in this examiner, it has been found that MMPs 14, 1, 2, 9 and uPA are highly overexpressed in patient specimens from multiple cancer sites including ones with highest overall incidence of positive margins (FIGS. 35, 36). Consequently, novel protease-specific targeting molecules developed within this proposal for surgical guidance and modulation of ionizing radiation can potentially be also used to improve outcome in these other cancer sites with high unmet clinical need.

### C2. Experimental Design

### C2.1-Specific Aim 1. Rational Design of Novel Protease-Selective Substrates

a: Use rational design to develop new peptide substrates that are specific for cleavage by MMP14, MMP2, MMP1, MMP11, MMP9 and uPA. There are 5 promising peptide substrate candidates for 4 different proteases with high specificity (MMP14, MMP2, MMP9 and uPA). This example will focus on characterizing these initial promising substrate candidates and extent our strategy to other substrates.

b: Incorporate these peptide substrates into our FRET based RACPP design and evaluate enzyme kinetics

Problem being Addressed:

RACPPs are capable of monitoring protease activity in real time living animal models of cancer. Our first generation RACPP, although good enough to have received FDA approval for ongoing clinical testing, shows non-specific cleavage by multiple MMPs. To tease out the involvement of specific individual proteases involved in the various stages of cancer growth, invasion and metastasis, it was sought to identify RACPP cleavable sites that are specific for individual select proteases.

Experimental Details:

Using TCGA, extracellular proteases were classified by their differential mRNA expression between tumor and paired normal tissue¹⁵. Several MMPs were identified (14, 1, 2 and 9, 11 FIG. 35) and uPA (FIG. 36) as showing highest differential expression between cancer and paired normal tissue for HNC and many other cancers. Next, it was sought to identify amino acids sequences that might be cleaved specifically by these proteases.

To generate new substrates for MMPs, amino acids were substituted at key position at and near the cleavage site based on the following rule derived by recent work by Ratnikov et al²⁶ that the amino acid at a certain position must improve cleavage by the specific enzyme and reduce cleavage by other related enzymes. For increased specificity, an amino acid should be red for the enzyme of interest compared green for the other enzymes at the same substitution position (FIG. 36A). These substrates will be tested by treating 5 μM peptide with 20 nM of each enzyme for 2 hours and analyse by gel electrophoresis.

To generate novel RACPP species that are specific for uPA, a set of protease substrates were screened that were diverse and likely cleaved by serine protease in that they contain either a lysine or arginine residue flanked by diverse amino acids.

### Preliminary Results:

MMP Selective Substrates:

Our current best MMP14-selective substrate is derived from a previously reported substrate which was selective for MMP-14 over MMP-2 and 9 but was inefficiently cleaved²³. This novel substrate, RSHG-(homoF)-FLY (SEQ ID NO: 70), was generated using site specific substitution based on consensus cleavage sequences reported by Ratnikov et al. and is highly selective and efficiently cleaved by MMP-14 over other MMPs. Our current best MMP2 selective substrate (TIAH/LH) is selectively cleaved by MMP-2 versus 9 and 14. There are two MMP9 selective substrates, SNPYK-Y (SEQ ID NO: 21) and SNPYG-Y (SEQ ID NO: 23). FAM/Cy5 ratiometric versions of RACPPs with these sequences were made and tested to confirm selectivity. The preferred form Cy5/Cy7 ratiometric versions for in vivo testing with a pegylated carrier to help with solubility is currently being synthesized. For MMP 11 a similar approach will be adopted in finding selective substrates.²⁶.

uPA Selective Substrates:

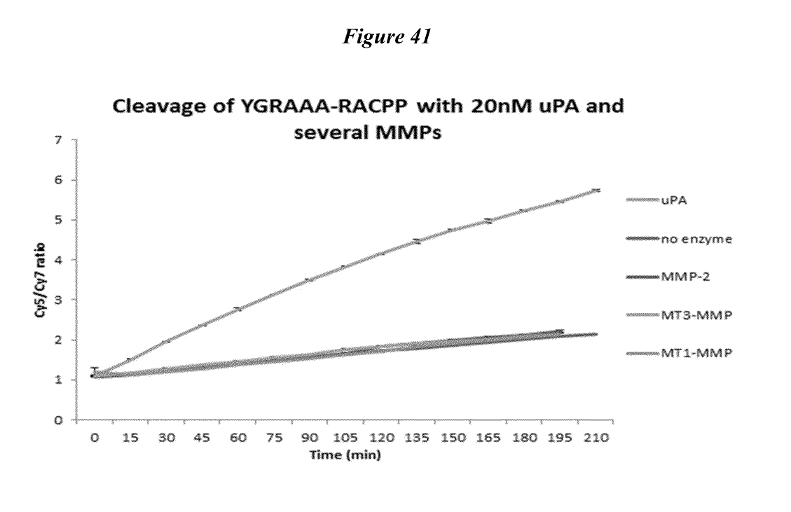

A substrate with sequence YGRAAA (SEQ ID NO: 17) was found to be efficiently cleaved by uPA and to a lesser extent by tPA. Although this cleavage sequence demonstrates specificity for uPA compared to MMPs (FIG. 41), a goal is to further optimize cleavage kinetics with additional sequential peptide substitutions and testing against an extended panel of cancer-associated proteases.

Potential Pitfall:

Designing a completely specific substrate can be very challenging since enzymes are known to be promiscuous and the substrate may be cleaved to a small extent by other enzymes. This is especially true for highly related enzymes like MMP2 and 9.

To assure specificity, time course measurements will be performed with the fluorescence plate reader and determine Michaelis Menton kinetics rates. Our substrate candidates will proceed to in vivo testing if they are cleaved by the intended MMP at a 10 fold higher rate than the other related MMPs. To be certain that a given protease is specifically cleaving a given substrate, RNA interference will be used to knock down each protease or explore using murine knockout of a given protease.

c2.2-Specific Aim 2. Determine Sensitivity/Specificity of Novel Protease-Selective RACPPs for Tumor Detection During Surgery, Ex Vivo Pathological Examination of Surgical Specimens, Ex Vivo Screening Assay to Determine Individual Protease Activity for Banked Tissues.

a)Evaluate specificity and sensitivity of MMP14, 2, 9, 1, 11 or uPA-selective RACPPs for tumor margin and lymph node metastasis detection in vivo

b) Evaluate specificity and sensitivity of MMP14, 2, 1, 9, 11 or uPA-selective RACPPs for tumor margin and lymph node metastasis detection ex vivo in surgical specimens

d)Test tumor-free survival following surgery with and without protease-selective RACPP fluorescence guidance

c) Develop ex vivo screening assay to determine individual selective protease activity for banked tumor samples and correlate with clinical stage and outcome

Problem being Addressed:

Nonspecific cleavage of previously described MMP sensitive probes has been shown to give rise to high uptake in non-cancer tissue such as tissues nearly injury sites, inflammation and cartilage^{19,34,35}. Furthermore, a given patient derived tumor may have different protease activity profile compared. The panel of RACPPs will be used to develop in SA1 with high specificity for a given protease to systematically evaluate their cancer sensitivity and specificity in surgical specimens compared to gold standard evaluation using H&E. It is anticipated that development of panel of protease-selective RACPPs will enable precise reporting for a given patient tumor profile and thus better correlation with clinical stage and outcome

### Experimental Details:

Tumor detection in vivo: To evaluate level of specific protease activity in tumor vs. surrounding tissue and ability to improve surgical margin detection, tumor-bearing will be injected mice intravenously (IV) with the various protease-selective RACPPs and noncleavable controls. In vivo apparent tumor vs. adjacent normal tissue will be documented with ratiometric fluorescence and white light reflectance imaging using a customized fluorescence dissecting surgical scope as previously described¹⁷. The mice will then be sacrificed according to UCSD approved protocol; apparent tumors and adjacent normal tissue will be harvested, cryosectioned and ratiometric fluorescence imaging for protease activity measured. Sensitivity and specificity will be evaluated for tumor identification using a receiver operator curve against the gold standard of H&E analysis for each protease-selective probe on multiple double-hit and single-hit xenografts.

Tumor Detection Ex Vivo:

Tumor bearing mice will be injected with various protease-selective RACPPs. Tumors will be excised using white light reflectance alone. Ex vivo pathological examination of tissues will be performed first with RACPP fluorescence imaging to identify foci of high uptake. Presence or absence of cancer will be confirmed with H&E. Receiver operator curve will be generated for each protease-selective probe.

Metastatic Lymph Node Detection:

To evaluate level of specific protease activity in metastatic lymph nodes vs. normal lymph nodes, tumor-bearing mice will be injected with cervical metastasis (generated as previously described¹⁷) intravenously (IV) with the various protease-selective RACPPs. Ratiometric fluorescence will be documented for every cervical lymph node as previously described¹⁷. The mice will then be sacrificed according to UCSD approved protocol; all cervical nodes harvested, cryosectioned and ratiometric fluorescence imaging for protease activity measured on cryosections. Sensitivity and specificity will be evaluated for cancer invasion for a given lymph node using a receiver operator curve against the gold standard of H&E analysis for each protease-selective probe.

Evaluate Tumor Free Survival Following RACPP Guided Surgery:

This example builds on prior extensive experience with ACPP guided surgery to evaluate tumor free HNC survival following RACPP guided surgery. The best performing novel MMP-cleavable RACPPs will be individually tested and the best performing uPA-cleavable RACPPs in different xenograft models of HNC-tumor bearing mice. Following surgery with either RACPP guidance or white light reflectance only, mice will be monitored for tumor free survival over 6 months as previously described by our group. It is hypothesized that 1) tumor free survival following surgery with RACPP guidance will be improved compared to surgery with white light reflectance alone; 2) the RACPP probe with highest sensitivity/specificity for tumor margin detection identified in SA2a will result in the best tumor free survival following RACPP guided surgery.

Ex Vivo Screening Assay to Determined Activity of Selected Proteases in Banked Tissue:

Banked frozen tissue will be thawed and gently homogenized in PBS, while kept cooled on ice to minimize the release of intracellular and intraorganellar proteases. Homogenates will then be microcentrifuged at 14,000×g for 1 minute, and supernatants (extracts) will be tested for ability to increase the Cy5 donor:Cy7 acceptor emission ratio of a protease-selective RACPPs. Testing of MMP14,2,9, uPA, MMP1, 11 in this order if tissue availability is limiting will be prioritized. Data will be correlated with existing documentation regarding patient stage and clinical outcome.

Preliminary Results:

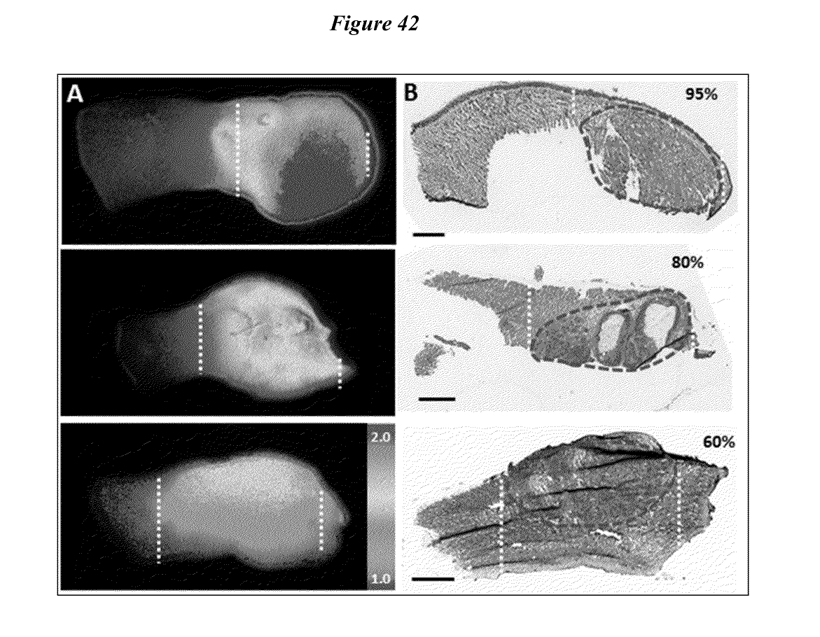

Increased ratiometric fluorescence signal using the first generation cleavage site of PLGC(me)AG (SEQ ID NO: 2) with increasing tumor burden has been demonstrated (FIG. 42). FAM/Cy5 FRET RACPPs that are MMP2, MMP 9 or MMP14 selective were recently generated. The FAM/Cy5 versions are simpler to synthesize using an automated peptide synthesizer and are useful for running a preliminary screen of the efficacy toward protease activity. In Ca127 tumor-bearing mice a prominent signal enhancement is seen with the MMP9 selective imaging probes compared to our previously reported PLGC(me)AG (SEQ ID NO: 2) RACPP. The tumor tissue have been preserved in OCT for analysis of localized uptake and will be quantified.

Potential Pitfalls:

To ensure that cancer cell lines used are representative of TCGA tumor specimens, mRNA expression profiles of various proteases of readily available HNC cell lines were measured. It was found that the majority of cell lines tested showed high MMP14, 1, 2 and uPA mRNA expression, suggesting that they are good representation of TCGA human tumor specimens.

### C2.3-Specific Aim 3. Determine Efficacy of Conformal IR Dosing Based on Protease-Selective RACPPs Imaging

a) Evaluate correlation of protease dependent RACPP signal with differential sensitivity to ionizing radiation

b) Test in vivo tumor control of protease-responsive RACPP linked with radiosensitizer

Problem being Addressed:

Radiotherapy is a mainstay treatment modality for 1-INC, and IMRT has the benefit of reducing long-term morbidity through highly conformal ionizing radiation delivery. Optimal parameters for defining the tumor target vs. adjacent tissue remain a clinical challenge. The hypothesis to be tested is whether IR dose thresholds can be individualized based upon level and localization of protease cleavable RACPP uptake.

Preliminary Data:

RACPP Localization to Highest Tumor Burden and Most Aggressive Tumors:

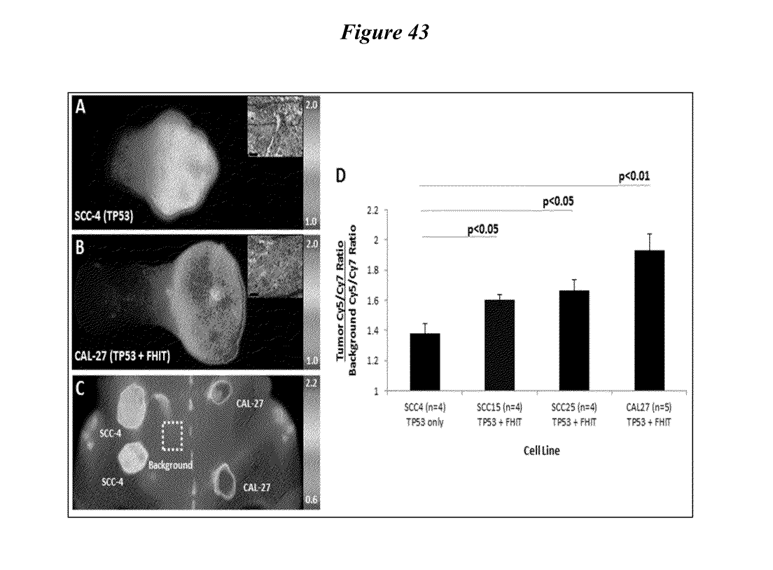

It was previously shown that highest RACPP localization to xenografts with highest tumor burden (FIG. 42)¹⁵. It was previously shown that more aggressive double-hit tumor xenografts (Ca127, SCC15, SCC25) have significantly higher MMP activity compared to less aggressive single-hit tumor xenografts (SCC4, FIG. 43). Finally, it was shown that the same double-hit xenografts have less radiosensitivity compared to the single hit xenografts (FIG. 43A). When RNA inteference was used to convert the single-hit cell line SCC4 into the double-hit genotype (FIG. 43B), a decrease in radiosensitivity compared to the wild type cell line was seen (FIG. 43C)¹⁸. Taken together, this data suggest that protease-cleavable RACPPs can be conjugated to radiosensitizers for targeted delivery to the areas of highest tumor burden or most aggressive tumor cells.

Table of Protease-Selective Substrates:

Individual proteases and their respective optimal cleavage substrates which were empirically derived.

TABLE 2
Table of protease-selective substrates:

| Protease | Cleavage |
|---|---|
| Thrombin | D/P/PRSFL |
| | (SEQ ID NO: 13; |
| | SEQ ID NO: 14) |
| | Nle-TPRSFL |
| | (SEQ ID NO: 15) |
| MMP2/9 | PLGC(Me)AG |
| | (SEQ ID NO: 2) |
| plasminogen | YGRAAA |
| Activators | (SEQ ID NO: 17) |
| Chymase | GVAYISGA |
| | (SEQ ID NO: 16) |
| Elastase | RLQLK(Ac)L |
| | (SEQ ID NO: 26) |
| | (Nle(O-Bzl)- |
| | Met(O)2-Oic-Abu) |
| MMP12 | PLGLEAA |
| | (SEQ ID NO: 30) |
| ACE/Renin | DRVYIHP |
| | (SEQ ID NO: 67), |
| | DRVYIHPFHLLYYS |
| | (SEQ ID NO: 68), |
| | IHPFHLVIHT |
| | (SEQ ID NO: 69) |
| MMP2 | TLSE-LH |
| | (SEQ ID NO: 24) |
| | TIAHLA |
| | (SEQ ID NO: 25) |
| MMP9 | SNPYK-Y |
| | (SEQ ID NO: 21) |
| | SNPKG-Y |
| | (SEQ ID NO: 22) |
| | SNPYG-Y |
| | (SEQ ID NO: 23) |
| MMP14 | RSHP(Hfe)TLY |
| | (SEQ ID NO: 19) |
| | RSHG(Hfe)FLY |
| | (SEQ ID NO: 20) |
| Cathepsin K | KLRFSKQ |
| | (SEQ ID NO: 27) |

### REFERENCES FOR EXAMPLE 6

1 Bauvois, B. New facets of matrix metalloproteinases MMP-2 and MMP-9 as cell surface transducers: outside-in signaling and relationship to tumor progression. Biochim Biophys Acta 1825, 29-36, doi:10.1016/j.bbcan.2011.10.001 (2012).
2 Curino, A. et al. Detection of plasminogen activators in oral cancer by laser capture microdissection combined with zymography. Oral oncology 40, 1026-1032, doi:10.1016/j.oraloncology.2004.05.011 (2004).
3 Coussens, L. M., Fingleton, B. & Matrisian, L. M. Matrix metalloproteinase inhibitors and cancer: trials and tribulations. Science 295, 2387-2392, doi:10.1126/science.1067100 (2002).
4 Hamano, Y. et al. Physiological levels of tumstatin, a fragment of collagen IV alpha3 chain, are generated by MMP-9 proteolysis and suppress angiogenesis via alphaV beta3 integrin. Cancer Cell 3, 589-601 (2003).
5 Montel, V. et al. Altered metastatic behavior of human breast cancer cells after experimental manipulation of matrix metalloproteinase 8 gene expression. Cancer research 64, 1687-1694 (2004).
6 Somiari, S. B. et al. Circulating MMP2 and MMP9 in breast cancer—potential role in classification of patients into low risk, high risk, benign disease and breast cancer categories. Int J Cancer 119, 1403-1411 (2006).
Oshima, H. et al. Suppressing TGFbeta Signaling in Regenerating Epithelia in an Inflammatory Microenvironment Is Sufficient to Cause Invasive Intestinal Cancer. Cancer Res 75, 766-776 (2015).
8 Willis, A. L., Sabeh, F., Li, X. Y. & Weiss, S. J. Extracellular matrix determinants and the regulation of cancer cell invasion stratagems. Journal of Microscopy 251, 250-260, doi:10.1111/jmi.12064 (2013).
9 Perentes, J. Y. et al. Cancer cell-associated MT1-MMP promotes blood vessel invasion and distant metastasis in triple-negative mammary tumors. Cancer research 71, 4527-4538, doi:10.1158/0008-5472.can-10-4376 (2011).
10 Uloza, V., Liutkevicius, V., Pangonyte, D., Saferis, V. & Lesauskaite, V. Expression of matrix metalloproteinases (MMP-2 and MMP-9) in recurrent respiratory papillomas and laryngeal carcinoma: clinical and morphological parallels. Eur Arch Otorhinolaryngol 268, 871-878, doi:10.1007/s00405-011-1494-1 (2011).
11 Wittekindt, C., Jovanovic, N. & Guntinas-Lichius, O. Expression of matrix metalloproteinase-9 (MMP-9) and blood vessel density in laryngeal squamous cell carcinomas. Acta Otolaryngol 131, 101-106, doi:10.3109/00016489.2010.506886 (2011).
12 Liu, W. W., Zeng, Z. Y., Wu, Q. L., Hou, J. H. & Chen, Y. Y. Overexpression of MMP-2 in laryngeal squamous cell carcinoma: a potential indicator for poor prognosis. Otolaryngology—head and neck surgery: official journal of American Academy of Otolaryngology-Head and Neck Surgery 132, 395-400, doi: 10.1016/j.otohns.2004.09.050 (2005).
13 Mallis, A., Teymoortash, A., Mastronikolis, N. S., Werner, J. A. & Papadas, T. A. MMP-2 expression in 102 patients with glottic laryngeal cancer. Eur Arch Otorhinolaryngol 269, 639-642, doi:10.1007/s00405-011-1625-8 (2012).
14 Zhou, C. X., Gao, Y., Johnson, N. W. & Gao, J. Immunoexpression of matrix metalloproteinase-2 and matrix metalloproteinase-9 in the metastasis of squamous cell carcinoma of the human tongue. Aust Dent J 55, 385-389, doi:10.1111/j.1834-7819.2010.01258.x (2010).
15 Hauff, S. J. et al. Matrix-metalloproteinases in head and neck carcinoma-cancer genome atlas analysis and fluorescence imaging in mice. Otolaryngology—head and neck surgery: official journal of American Academy of Otolaryngology-Head and Neck Surgery 151, 612-618, doi:10.1177/0194599814545083 (2014).
16 Kwaan, H. C., Mazar, A. P. & McMahon, B. J. The apparent uPA/PAI-1 paradox in cancer: more than meets the eye. Semin Thromb Hemost 39, 382-391, doi: 10.1055/s-0033-1338127 (2013).
17 Savariar, E. N. et al. Real-time in vivo molecular detection of primary tumors and metastases with ratiometric activatable cell-penetrating peptides. Cancer research 73, 855-864, doi:10.1158/0008-5472.CAN-12-2969 (2013).
18 Raju, S. C. et al. Combined TP53 mutation/3p loss correlates with decreased radiosensitivity and increased matrix-metalloproteinase activity in head and neck carcinoma. Oral oncology 51, 470-475, doi:10.1016/j.oraloncology.2015.01.014 (2015).
19 Nguyen, Q. T. et al. Surgery with molecular fluorescence imaging using activatable cell-penetrating peptides decreases residual cancer and improves survival. Proceedings of the National Academy of Sciences of the United States of America 107, 4317-4322, doi:10.1073/pnas.0910261107 (2010).
20 Hussain, T. et al. Surgical molecular navigation with a Ratiometric Activatable Cell Penetrating Peptide improves intraoperative identification and resection of small salivary gland cancers. Head & neck, doi:10.1002/hed.23946 (2014).
21 Metildi, C. A. et al. Ratiometric activatable cell-penetrating peptides label pancreatic cancer, enabling fluorescence-guided surgery, which reduces metastases and recurrence in orthotopic mouse models. Annals of surgical oncology 22, 2082-2087, doi:10.1245/s10434-014-4144-1 (2015).
22 Crisp, J. L. et al. Dual targeting of integrin alphavbeta3 and matrix metalloproteinase-2 for optical imaging of tumors and chemotherapeutic delivery. Molecular cancer therapeutics 13, 1514-1525, doi:10.1158/1535-7163.MCT-13-1067 (2014).
23 Buckel, L. et al. Tumor radiosensitization by monomethyl auristatin e: mechanism of action and targeted delivery. Cancer research 75, 1376-1387, doi:10.1158/0008-5472.CAN-14-1931 (2015).
24 Gross, A. M. et al. Multi-tiered genomic analysis of head and neck cancer ties TP53 mutation to 3p loss. Nature genetics 46, 939-943, doi:10.1038/ng.3051 (2014).
25 Rades, D. et al. Prognostic factors in head-and-neck cancer patients treated with surgery followed by intensity-modulated radiotherapy (IMRT), 3D-conformal radiotherapy, or conventional radiotherapy. Oral oncology 43, 535-543, doi:10.1016/j.oraloncology.2006.05.006 (2007).
26 Ratnikov, B. I. et al. Basis for substrate recognition and distinction by matrix metalloproteinases. Proceedings of the National Academy of Sciences of the United States of America 111, E4148-4155, doi:10.1073/pnas.1406134111 (2014).
27 Tsien, R. Y. & Harootunian, A. T. Practical design criteria for a dynamic ratio imaging system. Cell Calcium 11, 93-109 (1990).
28 Poeta, M. L. et al. TP53 mutations and survival in squamous-cell carcinoma of the head and neck. N Engl J Med 357, 2552-2561, doi:10.1056/NEJMoa073770 (2007).
29 Ohta, M. et al. The FHIT gene, spanning the chromosome 3p14.2 fragile site and renal carcinoma-associated t(3; 8) breakpoint, is abnormal in digestive tract cancers. Cell 84, 587-597 (1996).
30 Zanesi, N. et al. The tumor spectrum in FHIT-deficient mice. Proc Natl Acad Sci USA 98, 10250-10255, doi:10.1073/pnas.191345898 (2001).
31 Lee, J. I. et al. Loss of Fhit expression is a predictor of poor outcome in tongue cancer. Cancer Res 61, 837-841 (2001).
32 Egeblad, M. & Werb, Z. New functions for the matrix metalloproteinases in cancer progression. Nature reviews. Cancer 2, 161-174, doi:10.1038/nrc745 (2002).
33 Albright, C. F. et al. Matrix metalloproteinase-activated doxorubicin prodrugs inhibit HT1080 xenograft growth better than doxorubicin with less toxicity. Molecular cancer therapeutics 4, 751-760, doi:10.1158/1535-7163.mct-05-0006 (2005).
34 Olson, E. S. et al. In vivo characterization of activatable cell penetrating peptides for targeting protease activity in cancer. Integrative biology: quantitative biosciences from nano to macro 1, 382-393, doi:10.1039/b904890a (2009).
35 Olson, E. S. et al. Activatable cell penetrating peptides linked to nanoparticles as dual probes for in vivo fluorescence and MR imaging of proteases. Proceedings of the National Academy of Sciences of the United States of America 107, 4311-4316, doi:10.1073/pnas.0910283107 (2010).

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

1. An ex vivo method for detecting the presence of one or more protease activities in a neoplasia sample comprising
a) combining ex vivo said neoplasia sample from a subject with a molecule of the structure A-X-B-C, wherein
B is a peptide portion of about 5 to about 20 basic amino acid residues, which is suitable for cellular uptake,
A is a peptide portion of about 2 to about 20 acidic amino acid residues, which when linked with portion B is effective to inhibit or prevent cellular uptake of portion B, and
X is a cleavable linker of about 2 to about 100 atoms joining A with B, where X is cleavable under physiological conditions, and
C is a detectable moiety; and
b) detecting cleavage of A-X-B-C by detecting a change in said detectable moiety C, wherein said change in C is indicative of cleavage and said cleavage is indicative of the presence of one or more protease activities in said neoplasia.

2. An ex vivo method of determining a treatment regimen based on the protease profile of a neoplasia sample, comprising
a) combining ex vivo said neoplasia sample from a subject with a molecule of the structure A-X-B-C, wherein
B is a peptide portion of about 5 to about 20 basic amino acid residues, which is suitable for cellular uptake,
A is a peptide portion of about 2 to about 20 acidic amino acid residues, which when linked with portion B is effective to inhibit or prevent cellular uptake of portion B, and
X is a cleavable linker of about 2 to about 100 atoms joining A with B, where X is cleavable under physiological conditions and
C is a detectable moiety; and
b) detecting cleavage of A-X-B-C by detecting a change in detectable moiety C, wherein said change in C is indicative of cleavage and said cleavage is indicative of the presence of one or more protease activities and wherein the presence and/or absence of one or more protease activities allows for determining a medical treatment regimen.

3. The method of any of the preceding claims, wherein the medical regimen is a surgical regimen.

4. The method of any of the preceding claims, wherein the presence of the protease activity is indicative of neoplasia.

5. The method of any of the preceding claims, wherein the presence of the protease activity is indicative of metastasis.

6. The method of any of the preceding claims, wherein C is a fluorescent detectable moiety.

7. The method of any of the preceding claims, wherein C comprises a FRET pair.

8. The method of any of the preceding claims, said molecule further comprising a Q moiety, wherein when said Q moiety is present, said molecule has the structure Q-A-X-B-C.

9. The method of any of the preceding claims wherein C and Q comprise a FRET pair.

10. The method of any of the preceding claims wherein the FRET pair is selected from the group consisting of CFP:YFP; Cy5:Cy7; FITC:TRITC; Cy3:Cy5; EGFP:Cy3; EGFP:YFP; 6-FAM:LC Red 640 or Alexa Fluor 546; fluorescein:tetramethylrhodamine; IAEDANS:fluorescein; EDANS:Dabcyl; fluorescein:fluorescein; BODIPY FL:BODIPY FL; and fluorescein:QSY 7 and QSY 9.

11. The method of any of the preceding claims wherein cleavage of A-X-B-C is detected by FRET.

12. The method of any of the preceding claims wherein cleavage of Q-A-X-B-C is detected by FRET.

13. The method of any of the preceding claims, wherein said peptide portion A comprises about 5 to about 9 glutamates or aspartates.

14. The method of any of the preceding claims, wherein said peptide portion A comprises about 5 to about 9 consecutive glutamates or aspartates.

15. The method of any of the preceding claims, wherein said peptide portion B comprises about 9 to about 16 arginines.

16. The method of any of the preceding claims, wherein said peptide portion B comprises about 9 to about 16 consecutive arginines.

17. The method of any of the preceding claims, wherein said peptide portion A comprises D-amino acids.

18. The method of any of the preceding claims, wherein said peptide portion B comprises D-amino acids.

19. The method of any of the preceding claims, wherein said peptide portion A consists of D-amino acids.

20. The method of any of the preceding claims, wherein said peptide portion B consists of D-amino acids.

21. The method of any of the preceding claims, wherein said peptide portions A and B consists of D-amino acids.

22. The method of any of the preceding claims, wherein cleavable linker X is a flexible linker.

23. The method of any of the preceding claims, wherein cleavable linker X is a flexible linker about 6 to about 30 atoms in length.

24. The method of any of the preceding claims, wherein cleavable linker X is cleavable in an acidic environment.

25. The method of any of the preceding claims, wherein cleavable linker X comprises a peptide linkage.

26. The method of any of the preceding claims, wherein cleavable linker X comprises aminocaproic acid.

27. The method of any of the preceding claims, wherein cleavable linker X is configured for cleavage exterior to a cell.

28. The method of any of the preceding claims, wherein cleavable linker X is configured for cleavage by an enzyme.

29. The method of any of the preceding claims, wherein said enzyme is selected from the group consisting of a matrix metalloprotease, elastase, plasmin, thrombin, chymase, urokinase-type plasminogen activator and tissue plasminogen activator.

30. The method of any of the preceding claims, wherein cleavable linker X comprises an amino acid sequence selected from the group consisting of PLGLAG (SEQ ID NO: 1), PLGC(met)AG (SEQ ID NO: 2), EDDDDKA (SEQ ID NO: 3), RS-(Cit)-G-(homoF)-YLY (SEQ ID NO: 4), CRPAHLRDSG (SEQ ID NO: 5), SLAYYTA (SEQ ID NO: 6), NISDLTAG (SEQ ID NO: 7), PPSSLRVT (SEQ ID NO: 8), SGESLSNLTA (SEQ ID NO: 9), RIGFLR (SEQ ID NO: 10), RLQLA(acetyl)L (SEQ ID NO: 11), RLQLKL (SEQ ID NO: 12), DPRSFL (SEQ ID NO: 13), PPRSFL (SEQ ID NO: 14), Norleucine-TPRSFL (SEQ ID NO: 15), GVAY|SGA (SEQ ID NO: 16), YGRAAA (SEQ ID NO: 17), YGPRNR (SEQ ID NO: RSHP(Hfe)TLY (SEQ ID NO: 19), RSHG(Hfe)FLY (SEQ ID NO: 20), SNPYK-Y (SEQ ID NO: 21), SNPKG-Y (SEQ ID NO: 22), SNPYG-Y (SEQ ID NO: 23), TLSE-LH (SEQ ID NO: TIAHLA (SEQ ID NO: 25), (RLQLK(acetyl)L (SEQ ID NO: 26), and KLRFSKQ (SEQ ID NO: 27).

31. The method of any of the preceding claims, wherein cleavable linker X comprises a S-S linkage.

32. The method of any of the preceding claims, wherein cleavable linker X comprises a transition metal complex, wherein said transition metal complex linker is cleaved when the metal is reduced.

33. The method of any of the preceding claims, wherein in said method comprises multiple molecules of the structure A-X-B-C and wherein the cleavable linker X comprises a plurality of cleavable linkers X.

34. The method of any of the preceding claims, wherein the plurality of cleavable linkers X linking a portion A to a structure B-C are cleavable by a single protease.

35. The method of any of the preceding claims, wherein the plurality of cleavable linkers X linking a portion A to a structure B-C are cleavable by more than one protease.

36. An in vivo method of determining a treatment regimen based on the protease profile of a neoplasia, comprising
a) providing to a subject a molecule of the structure A-X-B-C, wherein
B is a peptide portion of about 5 to about 20 basic amino acid residues, which is suitable for cellular uptake,
A is a peptide portion of about 2 to about 20 acidic amino acid residues, which when linked with portion B is effective to inhibit or prevent cellular uptake of portion B, and
X is a cleavable linker of about 2 to about 100 atoms joining A with B, where X is cleavable under physiological conditions and
C is a detectable moiety; and
b) detecting cleavage of A-X-B-C by detecting a change in detectable moiety C, wherein said change in C is indicative of cleavage and said cleavage is indicative of the presence of one or more protease activities and wherein the presence and/or absence of one or more protease activities allows for determining a medical treatment regimen.

37. The method of any of the preceding claims, wherein the medical treatment regimen is a surgical regimen.

38. The method of any of the preceding claims, wherein the presence of the protease activity is indicative of neoplasia.

39. The method of any of the preceding claims, wherein cleavable linker X comprises an amino acid sequence selected from the group consisting of PLGLAG (SEQ ID NO: 1), PLGC(met)AG (SEQ ID NO: 2), EDDDDKA (SEQ ID NO: 3), RS-(Cit)-G-(homoF)-YLY (SEQ ID NO: 4), CRPAHLRDSG (SEQ ID NO: 5), SLAYYTA (SEQ ID NO: 6), NISDLTAG (SEQ ID NO: 7), PPSSLRVT (SEQ ID NO: 8), SGESLSNLTA (SEQ ID NO: 9), RIGFLR (SEQ ID NO: 10), RLQLA(acetyl)L (SEQ ID NO: 11), RLQLKL (SEQ ID NO: 12), DPRSFL (SEQ ID NO: 13), PPRSFL (SEQ ID NO: 14), Norleucine-TPRSFL (SEQ ID NO: 15), GVAY|SGA (SEQ ID NO: 16), YGRAAA (SEQ ID NO: 17), YGPRNR (SEQ ID NO: n, RSHP(Hfe)TLY (SEQ ID NO: 19), RSHG(Hfe)FLY (SEQ ID NO: 20), SNPYK-Y (SEQ ID NO: 21), SNPKG-Y (SEQ ID NO: 22), SNPYG-Y (SEQ ID NO: 23), TLSE-LH (SEQ ID NO: 24), TIAHLA (SEQ ID NO: 25), (RLQLK(acetyl)L (SEQ ID NO: 26), and KLRFSKQ (SEQ ID NO: 27).


SEQUENCE LISTING (95 sequences)

SEQ ID NO: 1 - protein, 6 aa, Artificial Sequence
  cleavable linker
PLGLAG

SEQ ID NO: 2 - protein, 6 aa, Artificial Sequence
  cleavable linker
PLGCAG

SEQ ID NO: 3 - protein, 7 aa, Artificial Sequence
  cleavable linker
EDDDDKA

SEQ ID NO: 4 - protein, 7 aa, Artificial Sequence
  cleavable linker
RSGFYLY

SEQ ID NO: 5 - protein, 10 aa, Artificial Sequence
  cleavable linker
CRPAHLRDSG

SEQ ID NO: 6 - protein, 7 aa, Artificial Sequence
  cleavable linker
SLAYYTA

SEQ ID NO: 7 - protein, 8 aa, Artificial Sequence
  cleavable linker
NISDLTAG

SEQ ID NO: 8 - protein, 8 aa, Artificial Sequence
  cleavable linker
PPSSLRVT

SEQ ID NO: 9 - protein, 10 aa, Artificial Sequence
  cleavable linker
SGESLSNLTA

SEQ ID NO: 10 - protein, 6 aa, Artificial Sequence
  cleavable linker
RIGFLR

SEQ ID NO: 11 - protein, 6 aa, Artificial Sequence
  cleavable linker
RLQLAL

SEQ ID NO: 12 - protein, 6 aa, Artificial Sequence
  cleavable linker
RLQLKL

SEQ ID NO: 13 - protein, 6 aa, Artificial Sequence
  cleavable linker
DPRSFL

SEQ ID NO: 14 - protein, 6 aa, Artificial Sequence
  cleavable linker
PPRSFL

SEQ ID NO: 15 - protein, 7 aa, Artificial Sequence
  cleavable linker
LTPRSFL

SEQ ID NO: 16 - protein, 7 aa, Artificial Sequence
  cleavable linker
GVAYSGA

SEQ ID NO: 17 - protein, 6 aa, Artificial Sequence
  cleavable linker
YGRAAA

SEQ ID NO: 18 - protein, 6 aa, Artificial Sequence
  cleavable linker
YGPRNR

SEQ ID NO: 19 - protein, 7 aa, Artificial Sequence
  cleavable linker
RSHPTLY

SEQ ID NO: 20 - protein, 7 aa, Artificial Sequence
  cleavable linker
RSHGFLY

SEQ ID NO: 21 - protein, 6 aa, Artificial Sequence
  cleavable linker
SNPYKY

SEQ ID NO: 22 - protein, 6 aa, Artificial Sequence
  cleavable linker
SNPKGY

SEQ ID NO: 23 - protein, 6 aa, Artificial Sequence
  cleavable linker
SNPYGY

SEQ ID NO: 24 - protein, 6 aa, Artificial Sequence
  cleavable linker
TLSELH

SEQ ID NO: 25 - protein, 6 aa, Artificial Sequence
  cleavable linker
TIAHLA

SEQ ID NO: 26 - protein, 6 aa, Artificial Sequence
  cleavable linker
RLQLKL

SEQ ID NO: 27 - protein, 7 aa, Artificial Sequence
  cleavable linker
KLRFSKQ

SEQ ID NO: 28 - protein, 6 aa, Artificial Sequence
  cleavable linker
PLGCAG

SEQ ID NO: 29 - protein, 11 aa, Artificial Sequence
  cleavable linker
CATKKLRFSKQ

SEQ ID NO: 30 - protein, 7 aa, Artificial Sequence
  cleavable linker
PLGLEEA

SEQ ID NO: 31 - protein, 6 aa, Artificial Sequence
  cleavable linker
SNPFKY

SEQ ID NO: 32 - protein, 7 aa, Artificial Sequence
  cleavable linker
KPRGSKQ

SEQ ID NO: 33 - protein, 7 aa, Artificial Sequence
  cleavable linker
KLRFSKQ

SEQ ID NO: 34 - protein, 7 aa, Artificial Sequence
  cleavable linker
KKPGSKQ

SEQ ID NO: 35 - protein, 6 aa, Artificial Sequence
  cleavable linker
HPGGPQ

SEQ ID NO: 36 - protein, 7 aa, Artificial Sequence
  cleavable linker
LTLRSLQ

SEQ ID NO: 37 - protein, 10 aa, Artificial Sequence
  cleavable linker
SGARGIKLTA

SEQ ID NO: 38 - protein, 7 aa, Artificial Sequence
  cleavable linker
RSGHYLY

SEQ ID NO: 39 - protein, 19 aa, Artificial Sequence
  peptide
EDDDDKAXRRRRRRRRRXC

SEQ ID NO: 40 - protein, 22 aa, Artificial Sequence
  peptide
XCRRRRRRRRRXEEEEEEEEEC

SEQ ID NO: 41 - protein, 17 aa, Artificial Sequence
  peptide
XCEEEEXRRRRRRRRRC

SEQ ID NO: 42 - protein, 23 aa, Artificial Sequence
  peptide
EEEEEDDDDKAXRRRRRRRRRXC

SEQ ID NO: 43 - protein, 19 aa, Artificial Sequence
  peptide
EDDDDKAXRRRRRRRRRXC

SEQ ID NO: 44 - protein, 22 aa, Artificial Sequence
  peptide
EEEEEDDDDKARRRRRRRRRXC

SEQ ID NO: 45 - protein, 19 aa, Artificial Sequence
  peptide
DDDDDDKARRRRRRRRRXC

SEQ ID NO: 46 - protein, 23 aa, Artificial Sequence
  peptide
EEDDDDKARXRRXRRXRRXRRXC

SEQ ID NO: 47 - protein, 7 aa, Artificial Sequence
  peptide
EDAXRXC

SEQ ID NO: 48 - protein, 16 aa, Artificial Sequence
  peptide
EDDDDKAXRRRRRRXC

SEQ ID NO: 49 - protein, 23 aa, Artificial Sequence
  peptide
EEEDDDEEEDAXRRRRRRRRRXY

SEQ ID NO: 50 - protein, 23 aa, Artificial Sequence
  peptide
EEEEEDDDDKAXRRRRRRRRRXC

SEQ ID NO: 51 - protein, 22 aa, Artificial Sequence
  peptide
EEEEEDDDDKARRRRRRRRRXC

SEQ ID NO: 52 - protein, 19 aa, Artificial Sequence
  peptide
EDDDDKAXRRRRRRRRRXC

SEQ ID NO: 53 - protein, 23 aa, Artificial Sequence
  peptide
EEDDDDKARXRRXRRXRRXRRXC

SEQ ID NO: 54 - protein, 19 aa, Artificial Sequence
  peptide
DDDDDDKARRRRRRRRRXC

SEQ ID NO: 55 - protein, 18 aa, Artificial Sequence
  DF4
DWFKAFYDKVAEKFKEAF

SEQ ID NO: 56 - protein, 11 aa, Artificial Sequence
  JNK Inhibitor VI
RPKRPTTLNLF

SEQ ID NO: 57 - protein, 16 aa, Artificial Sequence
  SC 3036
KKHTDDGYMPMSPGVA

SEQ ID NO: 58 - protein, 28 aa, Artificial Sequence
  NEMO-Binding Domain Binding Peptide
DRQIKIWFQNRRMKWKKTALDWSWLQTE

SEQ ID NO: 59 - protein, 26 aa, Artificial Sequence
  NF-kB SN50
AAVALLPAVLLALLAPVQRKRQKLMP

SEQ ID NO: 60 - protein, 29 aa, Artificial Sequence
  TIRAP Inhibitor Peptide
RQIKIWFNRRMKWKKLQLRDAAPGGAIVS

SEQ ID NO: 61 - protein, 4 aa, Artificial Sequence
  drug
LEHD

SEQ ID NO: 62 - protein, 4 aa, Artificial Sequence
  drug
LEHD

SEQ ID NO: 63 - protein, 4 aa, Artificial Sequence
  drug
IETD

SEQ ID NO: 64 - protein, 4 aa, Artificial Sequence
  drug
IETD

SEQ ID NO: 65 - protein, 4 aa, Artificial Sequence
  drug
LEHD

SEQ ID NO: 66 - protein, 4 aa, Artificial Sequence
  drug
LETD

SEQ ID NO: 67 - protein, 7 aa, Artificial Sequence
  cleavable linker
DRVYIHP

SEQ ID NO: 68 - protein, 14 aa, Artificial Sequence
  cleavable linker
DRVYIHPFHLLYYS

SEQ ID NO: 69 - protein, 10 aa, Artificial Sequence
  cleavable linker
IHPFHLVIHT

SEQ ID NO: 70 - protein, 8 aa, Artificial Sequence
  cleavable linker
RSHGFFLY

SEQ ID NO: 71 - protein, 8 aa, Artificial Sequence
  cleavable linker
RSQGFYLY

SEQ ID NO: 72 - protein, 6 aa, Artificial Sequence
  cleavable linker
TLAHLH

SEQ ID NO: 73 - protein, 6 aa, Artificial Sequence
  cleavable linker
TISHLH

SEQ ID NO: 74 - protein, 6 aa, Artificial Sequence
  cleavable linker
TLSHLH

SEQ ID NO: 75 - protein, 6 aa, Artificial Sequence
  cleavable linker
TIAHFH

SEQ ID NO: 76 - protein, 7 aa, Artificial Sequence
  cleavable linker
KPRGSKQ

SEQ ID NO: 77 - protein, 7 aa, Artificial Sequence
  clevable linker
KKPGSKQ

SEQ ID NO: 78 - protein, 6 aa, Artificial Sequence
  cleavable linker
HPGGPQ

SEQ ID NO: 79 - protein, 7 aa, Artificial Sequence
  cleavable linker
LTLRSLQ

SEQ ID NO: 80 - protein, 10 aa, Artificial Sequence
  cleavable linker
SGTIAHLATA

SEQ ID NO: 81 - protein, 10 aa, Artificial Sequence
  cleavable linker
SGSNPYGYTA

SEQ ID NO: 82 - protein, 10 aa, Artificial Sequence
  cleavable linker
SGSNPYKYTA

SEQ ID NO: 83 - protein, 8 aa, Artificial Sequence
  cleavable linker
RSQGFYLY

SEQ ID NO: 84 - protein, 7 aa, Artificial Sequence
  cleavable linker
RSGFYLY

SEQ ID NO: 85 - protein, 8 aa, Artificial Sequence
  cleavable linker
RSQGFYLY

SEQ ID NO: 86 - protein, 6 aa, Artificial Sequence
  cleavable linker
RSGLAG

SEQ ID NO: 87 - protein, 6 aa, Artificial Sequence
  cleavable linker
RPGLAG

SEQ ID NO: 88 - protein, 7 aa, Artificial Sequence
  cleavable linker
RSLGLAG

SEQ ID NO: 89 - protein, 8 aa, Artificial Sequence
  cleavable linker
RAHGHFLY

SEQ ID NO: 90 - protein, 8 aa, Artificial Sequence
  cleavable linker
RAHGHTLY

SEQ ID NO: 91 - protein, 8 aa, Artificial Sequence
  cleavable linker
RAHPHTLY

SEQ ID NO: 92 - protein, 6 aa, Artificial Sequence
  cleavable linker
YIPLVY

SEQ ID NO: 93 - protein, 6 aa, Artificial Sequence
  cleavable linker
SNPFKY

SEQ ID NO: 94 - protein, 6 aa, Artificial Sequence
  cleavable linker
NTFLHL

SEQ ID NO: 95 - protein, 6 aa, Artificial Sequence
  cleavable linker
ARGIKL